(19) United States
(12) Patent Application Publication
(10) Pub. No.: US 2025/0302747 A1
(43) Pub. Date: Oct. 2, 2025

(54) LIPIDS, NANOPARTICLES COMPRISING THE SAME AND USES THEREOF

(71) Applicant: Academia Sinica, Taipei (TW)

(72) Inventors: Wen-Shan LI, Taipei City (TW); Han-Chung WU, Taipei City (TW); Jhih-Liang HUANG, Nantou County (TW); Shih-Chieh SU, New Taipei City (TW); Hsiu-Ting LIN, New Taipei City (TW)

(21) Appl. No.: 18/864,006

(22) PCT Filed: Jun. 8, 2023
(86) PCT No.: PCT/US2023/024788

§ 371 Date: Nov. 8, 2024

Related U.S. Application Data
(60) Provisional application No. 63/350,215, filed on Jun. 8, 2022.

(51) Int. Cl. A61K 9/1272; A61K 39/00; A61K 39/12; A61K 39/215; C07C 229/16

(52) U.S. Cl. CPC A61K 9/1272; A61K 39/12; A61K 39/215; C07C 229/16; A61K 2039/53

(57) ABSTRACT

Disclosed herein are novel lipids, lipid nanoparticlcs and their uses for the transport of therapeutic agents to a subject, or for the treatment and/or prophylaxis of diseases in the subject.

### CROSS-REFERENCE TO RELATED APPLICATIONS

This application claims priority and the benefit of U.S. Provisional Patent Application No. 63/350,215, filed Jun. 8, 2022, the entirety of which is incorporated herein by reference.

### BACKGROUND OF THE INVENTION

### 1. Field of the Invention

The present disclosure in general relates to the field of delivery of medicines, particularly, relates to nanoparticles formed by novel lipids for the transport of therapeutic agents such as nucleic acids, and to the use thereof in the treatment and/or prophylaxis of diseases.

### 2. Description of Related Art

Of the various reagents used to transfect cells with bioactive agents such as nucleic acids, those based on lipid nanoparticles (e.g., liposomes) mediated delivery are widely acknowledged to be the most effective. This is due mostly to their efficiency and ease of use. Lipid nanoparticles are artificially prepared spherical vesicles made of a lipid bilayer. To deliver the molecules to sites of action, the lipid bilayer can fuse with other bilayers such as the cell membrane, thus delivering the liposome contents inside the cell.

Lipid nanoparticles are used for drug deliver due to their unique properties. A lipid nanoparticle encapsulates a region of aqueous solution inside a hydrophobic membrane, dissolved hydrophilic solutes cannot readily pass through the lipids. Hydrophobic chemicals can be dissolved into the membrane, and in this way lipid nanoparticle can carry both hydrophobic molecules and hydrophilic molecules. Lipid nanoparticles can be combined with bioactive agents such as drugs, nucleic acids, and etc. and used to deliver these agents for the treatments and/or prophylaxis of diseases.

Recently, lipid nanoparticles have been utilized in COVID-19 mRNA vaccines. mRNA vaccines have proven to be effective for controlling COVID-19, and clinical trials have been carried out in many countries to evaluate mRNA vaccines against various other diseases. Advantages of mRNA vaccines are that they can induce adequate immune responses to protect hosts against infectious pathogens, and the vaccines are amenable to rapid manufacturing, which allows targeting of infectious pathogen variants. However, the low thermal stability of mRNA vaccines places serious limitations on their storage and distribution. For example, the mRNA vaccines for COVID-19 manufactured by Moderna and Pfizer-BioNTech can only be stored for 6 months at −20° C. and −80° C., respectively. Furthermore, the mRNA COVID-19 vaccine of Moderna is stable at room temperature only for 12 hours. Similarly, Pfizer-BioNTech mRNA COVID-19 vaccine is stable at room temperature only for 2 hours.

Accordingly, there exists in the related art a need of novel lipid molecules for the production of lipid nanoparticles for the delivery of therapeutic agents (e.g., nucleic acid).

### SUMMARY OF THE INVENTION

The present disclosure provides novel cationic lipids for forming nanoparticles for the non-viral transport of nucleic acids, and to the use thereof in the treatment and/or prophylaxis of diseases (e.g., an infection caused by a severe acute respiratory syndrome coronavirus 2 (SARS-CoV-2)).

The first aspect of the present disclosure pertains to a lipid of formula (I),

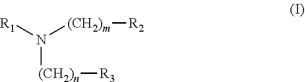

R₁ is alkyl or cycloalkyl optionally substituted with one or more hydroxyl, —CH₂OH,

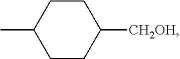

or —NR₂ groups;
m and n are independently an integral between 0 and 12;
R₂ and R₃ are independently H, alkeneyl, R^{a}, —(C═O)O(CH₂)R^{a}, —O(C═O)R^{b}, or —(C═O)OR^{b};
R^{a} is —CR′(COOR″)₂ or —CR′(COOR″)(COOR′″); and
R, R^{b}, R′, R″, and R′″ are independently H or alkyl.

According to embodiments of the present disclosure, wherein the lipid of formula (I) may be any one of,

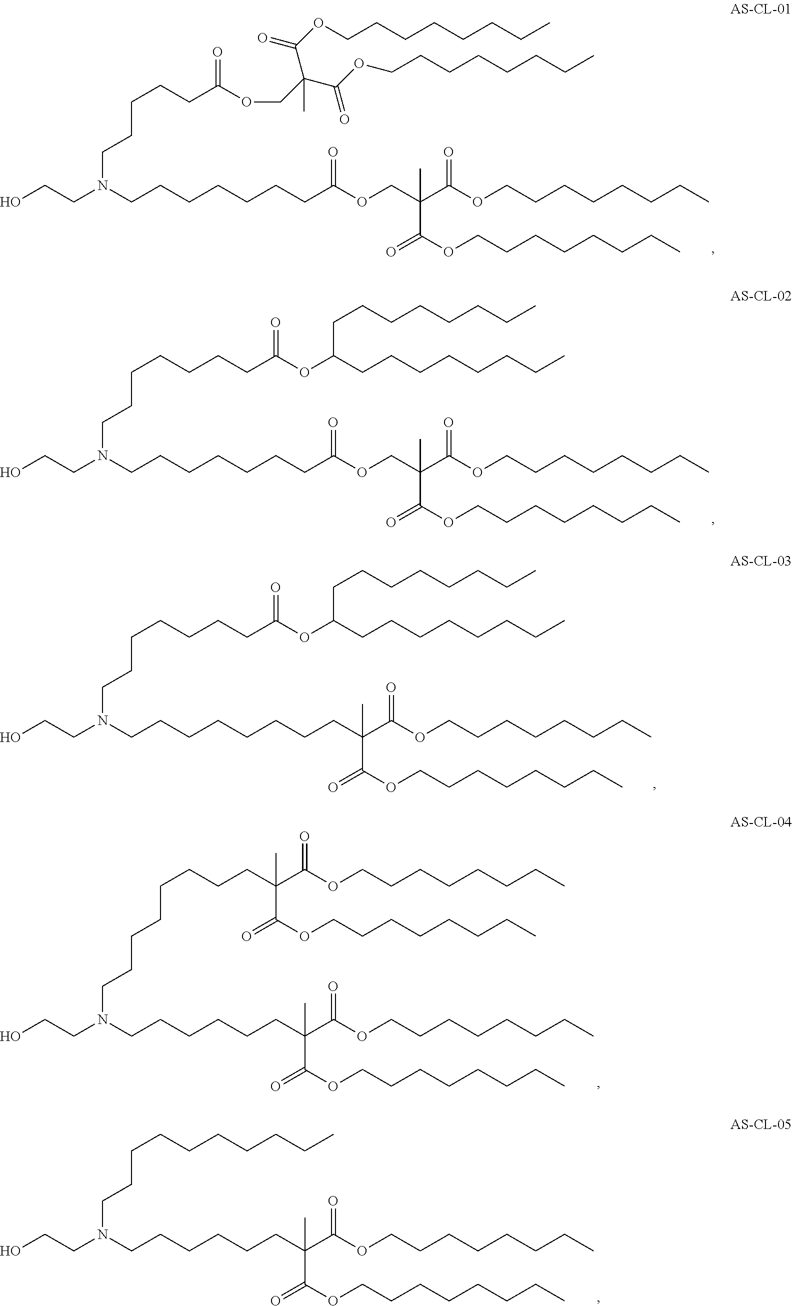 [SMILES: CCCCCCCCOC(=O)C(C)(COC(=O)CCCCCCCN(CCO)CCCCCC(=O)OCC(C)(C(=O)OCCCCCCCC)C(=O)OCCCCCCCC)C(=O)OCCCCCCCC ; CCCCCCCCOC(=O)C(C)(COC(=O)CCCCCCCN(CCO)CCCCCCCC(=O)OC(CCCCCCCC)CCCCCCCC)C(=O)OCCCCCCCC ; CCCCCCCCOC(=O)C(C)(CCCCCCCCN(CCO)CCCCCCCC(=O)OC(CCCCCCCC)CCCCCCCC)C(=O)OCCCCCCCC ; CCCCCCCCOC(=O)C(C)(CCCCCCCCN(CCO)CCCCCCC(C)(C(=O)OCCCCCCCC)C(=O)OCCCCCCCC)C(=O)OCCCCCCCC ; CCCCCCCCCCN(CCO)CCCCCCC(C)(C(=O)OCCCCCCCC)C(=O)OCCCCCCCC]

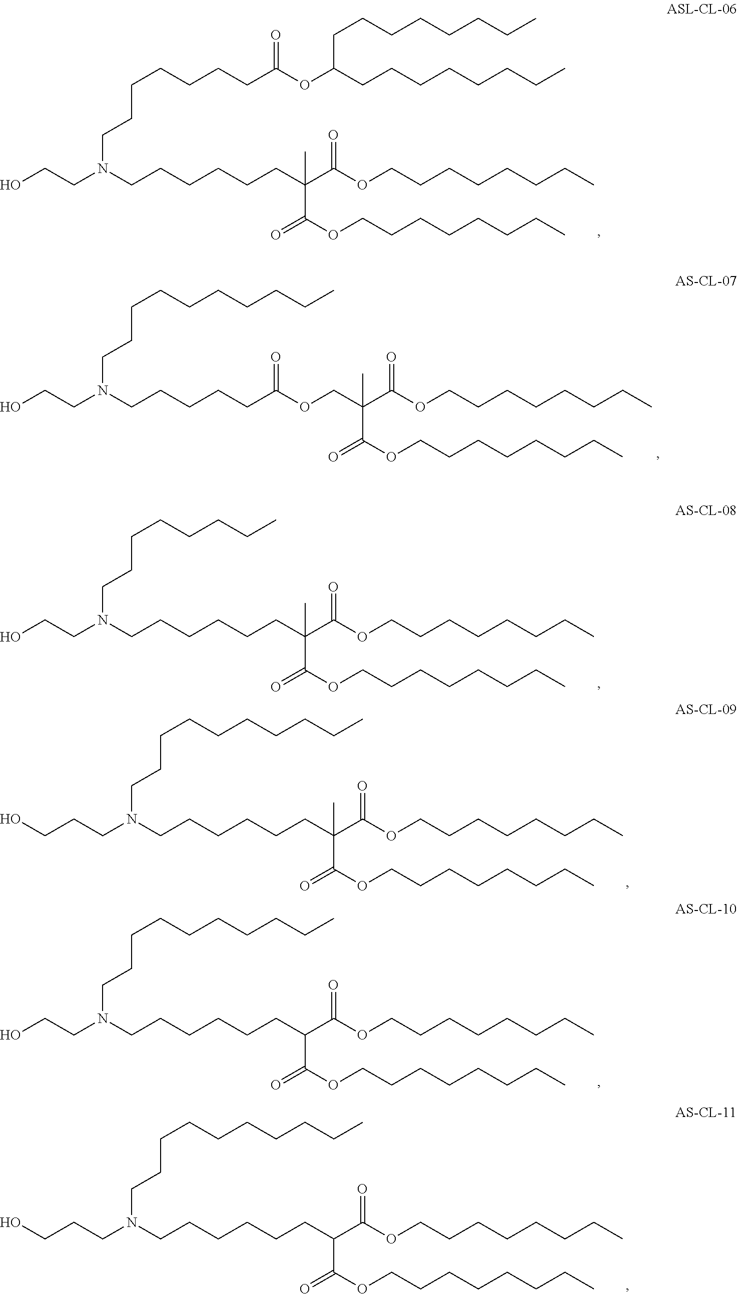

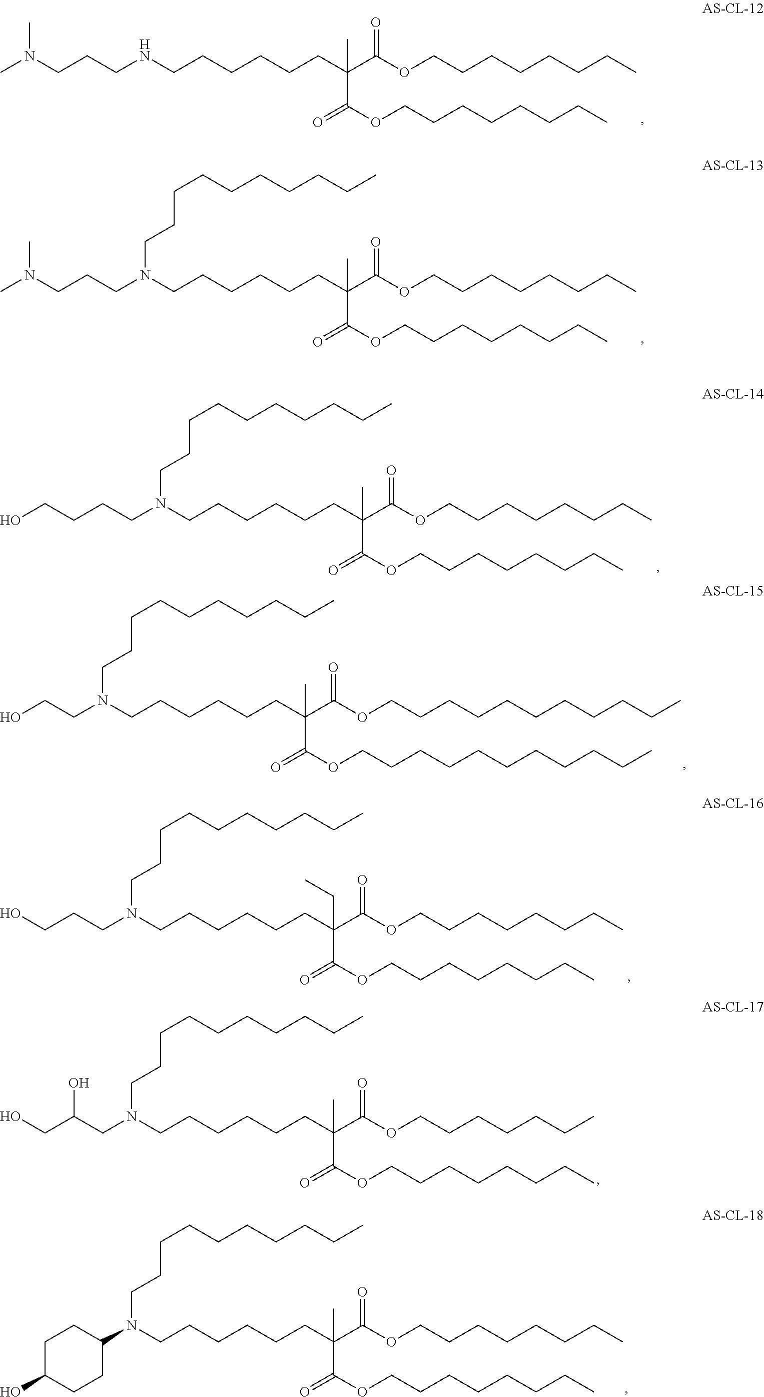

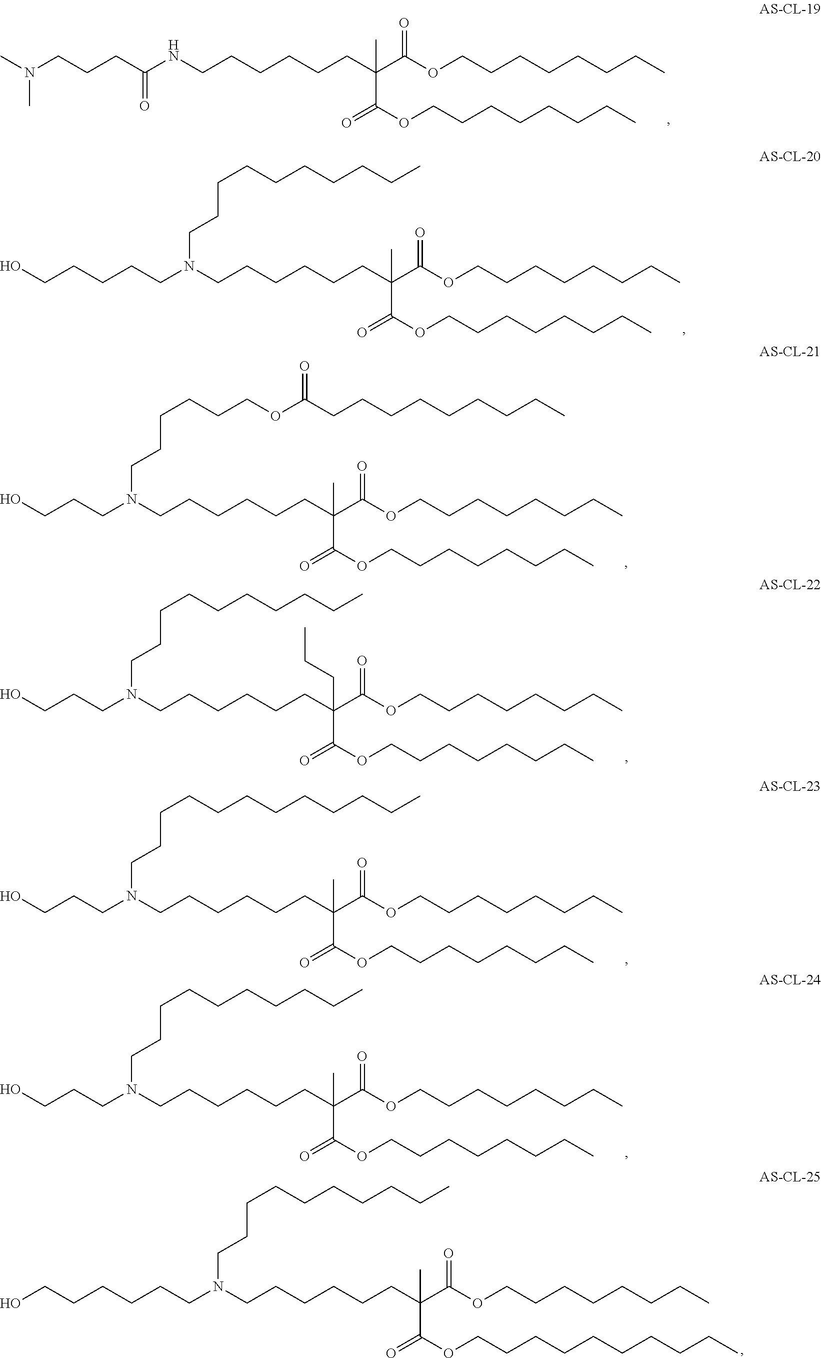

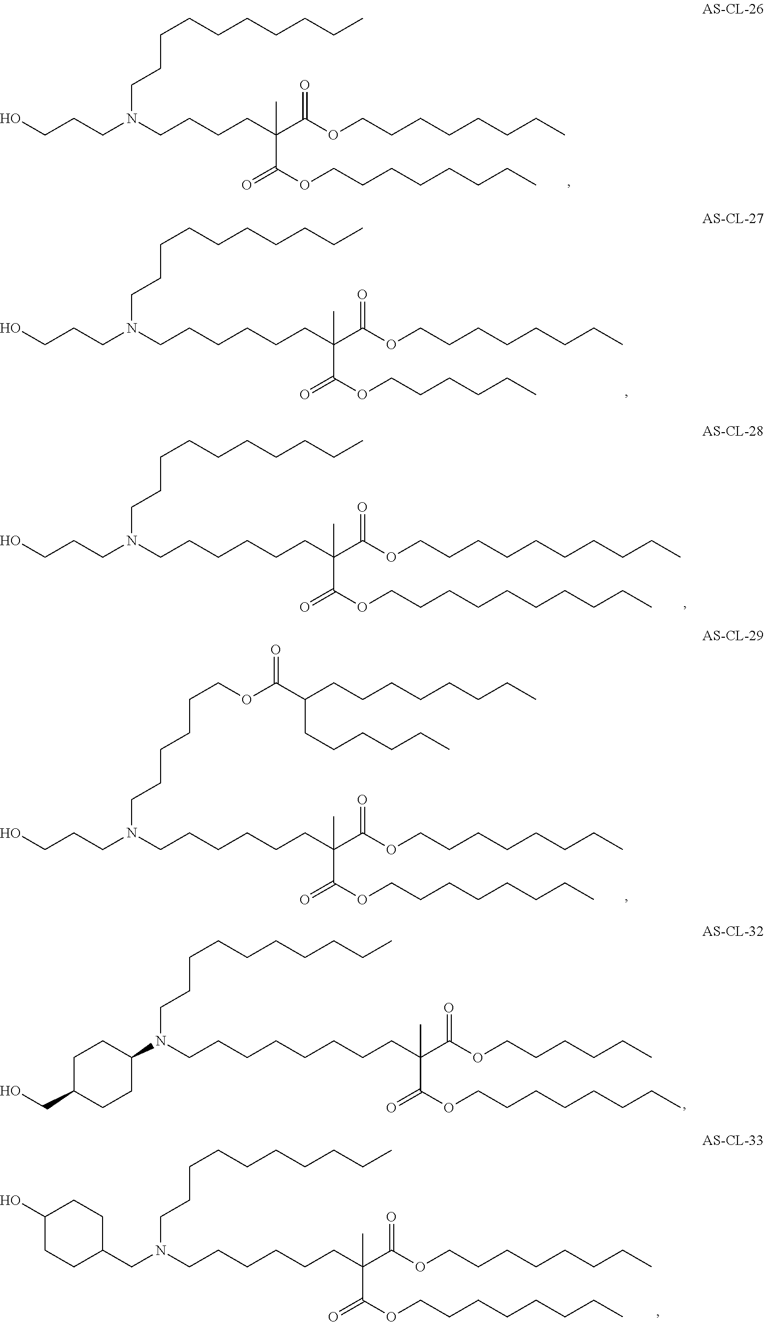

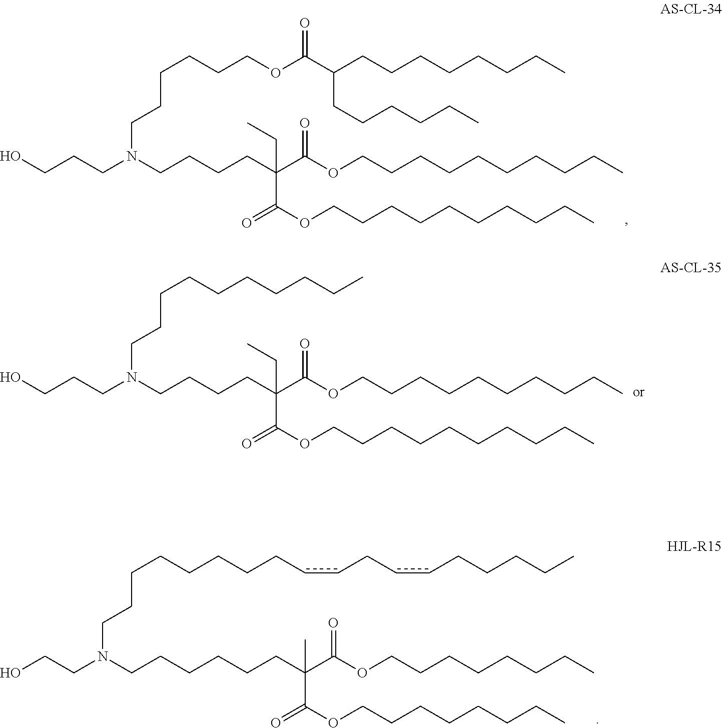

The second aspect of the present disclosure pertains to a lipid of formula (II),

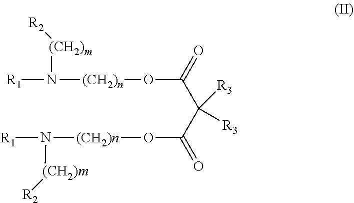

wherein,
R₁ and R₃ are independently alkyl optionally substituted with one or more hydroxyl group;
R₂ is H or —O(C═O)R′, in which R′ is alkyl; and
m and n are independently an integral between 1 to 10.

According to embodiments of the present disclosure, wherein the lipid of formula (II) may be any one of,

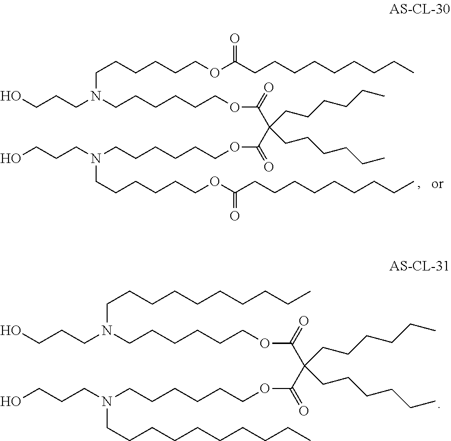 [SMILES: CCCCCCCCCC(=O)OCCCCCCN(CCCO)CCCCCCOC(=O)C(CCCCCC)(CCCCCC)C(=O)OCCCCCCN(CCCO)CCCCCCOC(=O)CCCCCCCCC ; CCCCCCCCCCN(CCCO)CCCCCCOC(=O)C(CCCCCC)(CCCCCC)C(=O)OCCCCCCN(CCCO)CCCCCCCCCC]

The third aspect of the present disclosure pertains to a lipid of formula (III),

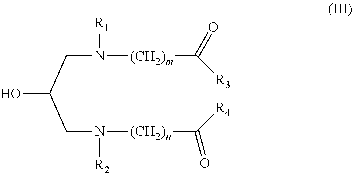

wherein,
m and n are independently an integral between 1 to 10; and
R₁, R₂, R₃ and R₄ are independently H or alkyl.

According to embodiments of the present disclosure, wherein the lipid of formula (III) has the structure of,

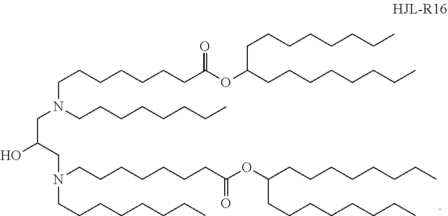 [SMILES: CCCCCCCCC(CCCCCCCC)OC(=O)CCCCCCCN(CCCCCCCC)CC(O)CN(CCCCCCCC)CCCCCCCC(=O)OC(CCCCCCCC)CCCCCCCC]

In further aspect, there is provided a lipid nanoparticle formed by one or more lipids of the present disclosure for the delivery of an active ingredient of interest (e.g., nucleic acids of a target protein or a therapeutic agent). The lipid nanoparticle comprises in its structure, a hydrophilic core; and an outer lipid bilayer shell formed by one or more lipids of formula (I) to (III).

Additionally or optionally, the lipid nanoparticle of the present disclosure further includes a therapeutic agent disposed in the hydrophilic core or in the outer lipid bilayer shell of the nanoparticle. The therapeutic agent will be in the hydrophilic core if hydrophilic or in the lipid shell if hydrophobic. The therapeutic agent may be a nucleic acid of a target protein.

Examples of the nucleic acid that may be encapsulated within the hydrophilic core of the present lipid nanoparticle include, but are not limited to, a double strand DNA (dsDNA), a single strand DNA (ssDNA), a small interference RNA (siRNA), a short hairpin RNA (shRNA), a messenger RNA (mRNA), a micro RNA (miRNA), a transfer RNA (tRNA) and a combination thereof. In some embodiments, the lipid nanoparticle of the present disclosure further includes mRNA of a severe acute respiratory syndrome coronavirus 2 (SARS-CoV-2) encapsulated in the hydrophilic core. In other embodiments, the lipid nanoparticle of the present disclosure further includes mRNA of an envelope (E) protein of a dengue virus encapsulated in the hydrophilic core.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The present description will be better understood from the following detailed description read in light of the accompanying drawings, where:

FIG. 1 Protein expression after administration of SARS-CoV-2 S protein (BA.5) mRNA-LNP complexes in vitro and in vivo. (A) Cellular ELISA data indicated the successful transfection of mRNA, (B) the binding activity of immunized sera to target evaluated by ELISA, and (C) Half-maximum inhibitory concentration (IC₅₀) for sera from immunized mice;

FIG. 2 Protein expression after administration of SARS-CoV-2 S protein (WT) mRNA-LNP complexes in vitro and in vivo. (A) Cellular ELISA data indicated the successful transfection of mRNA, (B) the binding activity of immunized sera to target evaluated by ELISA; and

FIG. 3 Functional analysis of DENV2 E mRNA-LNP complexes. (A) Protein expression level, and (B) Sera were collected from mice 6-weeks post first immunization dose, and dengue virus-specific antibody responses were analyzed by ELISA, and (C) Half-maximum inhibitory concentration (IC₅₀) for sera from immunized mice.

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

For the purposes of the present invention, lipid nanoparticles mean particles formed by a hydrophilic nucleus coated by a lipid outer shell, suitable for use in the treatment and/or prophylaxis of diseases, in which the active ingredient of interest (nucleic acid and/or therapeutic agent) will be in the hydrophilic nucleus if hydrophilic or in the lipid shell if hydrophobic.

For convenience, certain terms employed in the specification, examples and appended claims are collected here. Unless defined otherwise, all technical and scientific terms used herein have the same meaning as commonly understood by one of the ordinary skill in the art to which the present disclosure belongs.

Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

An atom, moiety, or group described herein may be unsubstituted or substituted, as valency permits, unless otherwise provided expressly. The term “optionally substituted” refers to substituted or unsubstituted. Unless otherwise indicated, the term “substituted.” when used to describe a chemical structure or moiety, refers to a derivative of that structure or moiety wherein one or more of its hydrogen atoms is substituted with one or more of atoms or groups other than hydrogen, such as halo, hydroxyl, alkyl, aryl, amino, alkylamino, and etc. The term “substituted” is contemplated to include substitution with all permissible substituents of organic compounds, any of the substituents described herein that results in the formation of a stable compound. The present invention contemplates any and all such combinations in order to arrive at a stable compound.

The term “alkyl” means a straight chain, branched hydrocarbon having from 1 to 20 (e.g., 1 to 10, 1 to 9, 1 to 8, 1 to 7, 1 to 6, 1 to 5, 1 to 4, 1 to 3, 1 to 2, or 1) carbon atoms. Examples of alkyl groups include methyl, ethyl, propyl, isopropyl, n-butyl, t-butyl, isobutyl, 2-isopropyl-3-methyl butyl, pentyl, pentan-2-yl, hexyl, isohexyl, heptyl, heptan-2-yl, 4,4-dimethylpentyl, octyl, 2,2,4-trimethylpentyl, nonyl, decyl, undecyl and dodecyl. Unless otherwise specified, each instance of alkyl is optionally substituted, i.e., unsubstituted (an “unsubstituted alkyl”) or substituted (a “substituted alkyl”) with one or more substituents. In certain embodiments, the alkyl group is unsubstituted C₁₋₂₁ alkyl. In one preferred example, the alkyl group is octyl (“—C₈H₁₇”). In another preferred example, the alkyl group is dodecyl (“—C₁₀H₂₁”). In other embodiments, the alkyl group is a substituted C₁₋₁₂ alkyl. In one preferred example, the alkyl group is ethyl substituted with one hydroxy group (“—C₂H₅OH”). In another preferred example, the alkyl group is propyl substituted with two hydroxy groups. In further examples, the alkyl is propyl substituted with an amino group (“—C₃H₇NH₂”). In still further preferred examples, the alkyl group is propyl substituted with dimethylamine (“—C₃H₆N(CH₃)₂).

“cycloalkyl” refers to a radical of a non-aromatic cyclic hydrocarbon group having from 3 to 10 ring carbon atoms (“C₃₋₁₀ cycloalkyl”) and zero heteroatoms in the non-aromatic ring system. In certain embodiments, the cycloalkyl group is either monocyclic (“monocyclic carbocyclyl”) or contain a fused, bridged, or spiro ring system such as a bicyclic system (“bicyclic alkyl”). In some embodiments, cycloalkyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. C Unless otherwise specified, each instance of a cycloalkyl group is independently optionally substituted, i.e., unsubstituted (an “unsubstituted cycloalkyl group”) or substituted (a “substituted cycloalkyl group”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The term “alkenyl” refers to a radical of a straight-chain or branched hydrocarbon group having from 2-20 carbon atoms, one or more carbon-carbon double bonds, and no triple bonds (“C₂₋₂₀ alkenyl”). In some embodiment, an alkenyl group has 18 carbon atoms (“C₁₈ alkenyl”) and one double bond (i.e., oleic acid). In some embodiments, an alkenyl group has 18 carbon atoms and two double bonds (i.e., linoleic acid). Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₂₀ alkenyl. In certain embodiments, the alkenyl group is substituted C₂₋₂₀ alkenyl.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Unless otherwise defined, all technical and scientific terms have the same meaning as commonly understood by one of ordinary skill in the art to which this invention pertains. In the case of conflict, the present document, including definitions will control.

### 1. Compounds

The compounds as described herein can have the structure of formula (I), which is described herein,

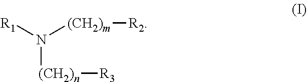

In formula (I), R₁ is alkyl or cycloalkyl optionally substituted with one or more hydroxyl, —CH₂OH,

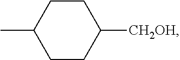

or —NR₂ groups. In some embodiments. R₁ is ethyl substituted with one hydroxyl group. In other embodiments, R₁ is ethyl substituted with two hydroxyl groups. In further embodiments, R₁ is ethyl substituted with dimethylamino group (i.e., —N(CH₃)₂). In some embodiments, R₁ is propyl substituted with one hydroxy group. In further embodiments, R₁ is propyl substituted with two hydroxy groups. In some embodiments, R₁ is hexyl substituted with one hydroxyl group. In other embodiments, R₁ is cyclohexyl substituted with one hydroxyl group. In further embodiments, R₁ is cyclohexyl substituted with —CH₂OH. In still further embodiments, R₁ is ethyl substituted with

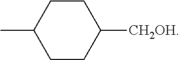

Additionally or alternatively, m and n are independently an integral between 0 and 12; and R₂ and R₃ are independently H, alkeneyl, R^{a}, —(C═O)O(CH₂)R^{a}, —O(C═O)R^{b}, or —(C═O)OR^{b}, in which R^{a} is —CR′(COOR″)₂ or —CR′(COOR″)(COOR′″), and R, R^{b}, R,′ R″, and R′″ are independently H or alkyl. In some embodiments, m is 5, n is 7, R₂ and R₃ are independently —(C═O)O(CH₂)R^{a}, in which R^{a} is —CR′(COOR″)₂, R′ is methyl, and R″ is —C₈H₁₇. In other embodiments, m and n are independently 7, R₂ is —O(C═O)R^{b}, and R₃ is —(C═O)O(CH₂)R^{a}, in which R^{a} is —CR′(COOR″)₂, and R^{b} is —CH(C₈H₁₇). In further embodiments, m is 10, n is 6, R₂ is H, and R₃ is R^{a}, which is —CR′(COOR″)₂, R′ is methyl, and R″ is —C₈H₁₇. In still further embodiments, m is 10, n is 6, R₂ is H, and R₃ is R^{a}, which is —CR′(COOR″)₂, R′ is methyl, and R″ is —C₈H₁₇. In some embodiments, m is 10, n is 6, R₂ is H, and R₃ is R^{a}, which is —CR′(COOR″)(COOR′″), R′ is —CH₃, R″ is —C₈H₁₇, and R′″ is —C₁₀H₂₁. In other embodiments, m is 0, n is 6, R₂ is —CH═CHCH₂CH═CH(CH2)₄CH₃, R₃ is R^{a}, which is —CR′(COOR″)₂, R′ is methyl, and R″ is —C₈H₁₇.

According to embodiments of the present disclosure, the lipid of formula (I) may be any one of,

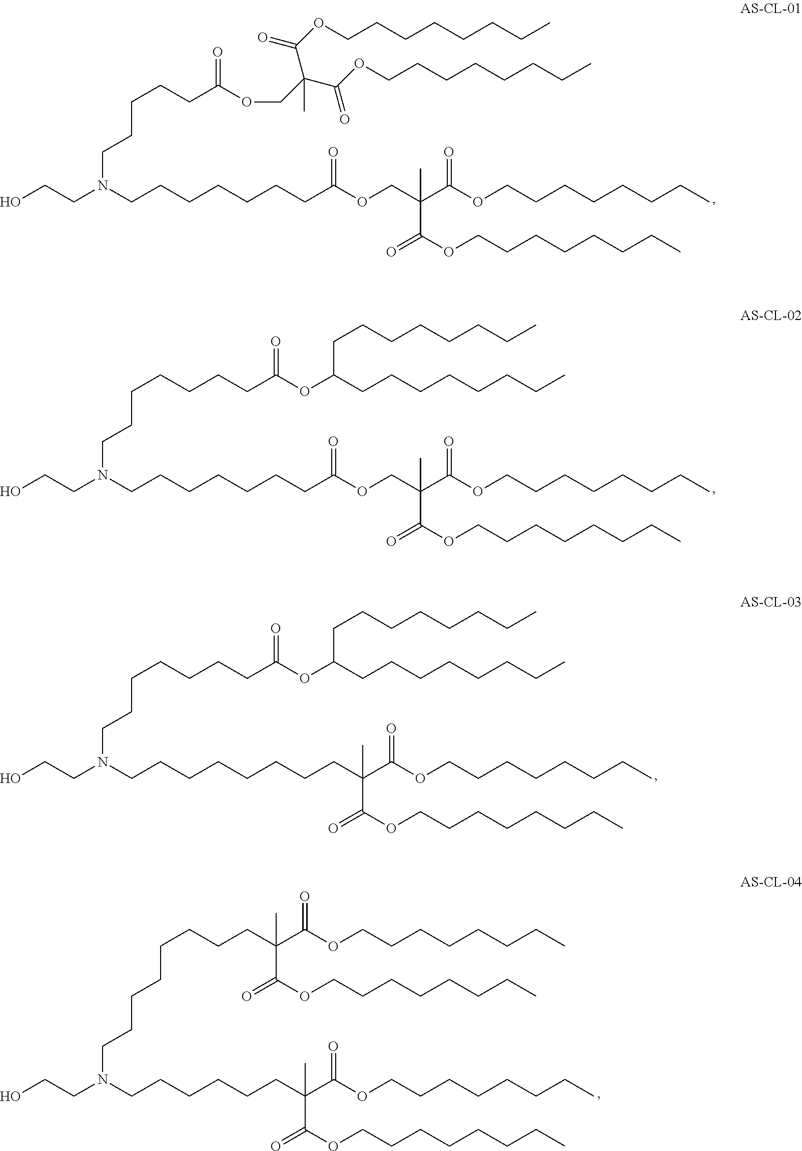 [SMILES: CCCCCCCCOC(=O)C(C)(COC(=O)CCCCCCCN(CCO)CCCCCC(=O)OCC(C)(C(=O)OCCCCCCCC)C(=O)OCCCCCCCC)C(=O)OCCCCCCCC ; CCCCCCCCOC(=O)C(C)(COC(=O)CCCCCCCN(CCO)CCCCCCCC(=O)OC(CCCCCCCC)CCCCCCCC)C(=O)OCCCCCCCC ; CCCCCCCCOC(=O)C(C)(CCCCCCCCN(CCO)CCCCCCCC(=O)OC(CCCCCCCC)CCCCCCCC)C(=O)OCCCCCCCC ; CCCCCCCCOC(=O)C(C)(CCCCCCCCN(CCO)CCCCCCC(C)(C(=O)OCCCCCCCC)C(=O)OCCCCCCCC)C(=O)OCCCCCCCC]

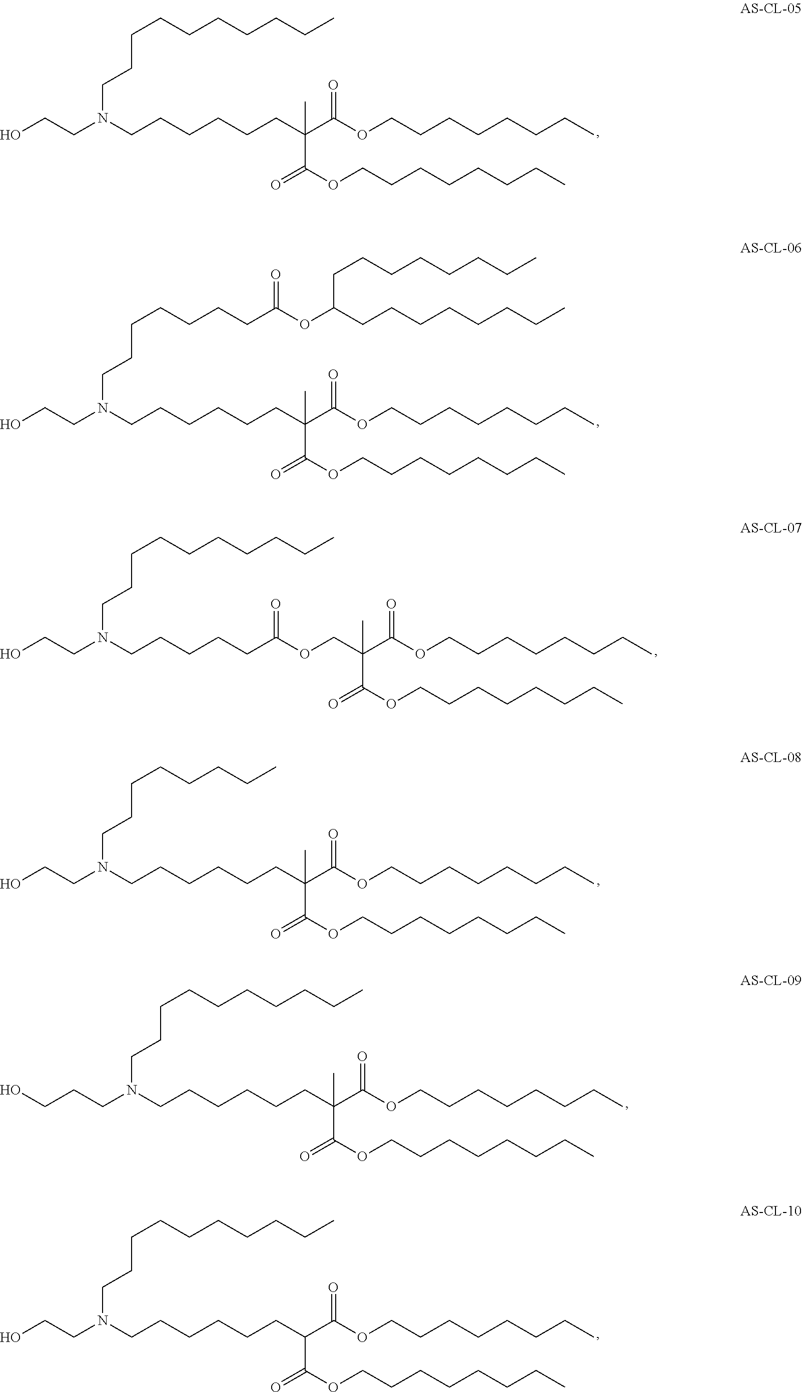 [SMILES: CCCCCCCCCCN(CCO)CCCCCCC(C)(C(=O)OCCCCCCCC)C(=O)OCCCCCCCC ; CCCCCCCCOC(=O)C(C)(CCCCCCN(CCO)CCCCCCCC(=O)OC(CCCCCCCC)CCCCCCCC)C(=O)OCCCCCCCC ; CCCCCCCCCCN(CCO)CCCCCC(=O)OCC(C)(C(=O)OCCCCCCCC)C(=O)OCCCCCCCC ; CCCCCCCCOC(=O)C(C)(CCCCCCN(CCO)CCCCCCCC)C(=O)OCCCCCCCC ; CCCCCCCCCCN(CCCO)CCCCCCC(C)(C(=O)OCCCCCCCC)C(=O)OCCCCCCCC ; CCCCCCCCCCN(CCO)CCCCCCC(C(=O)OCCCCCCCC)C(=O)OCCCCCCCC]

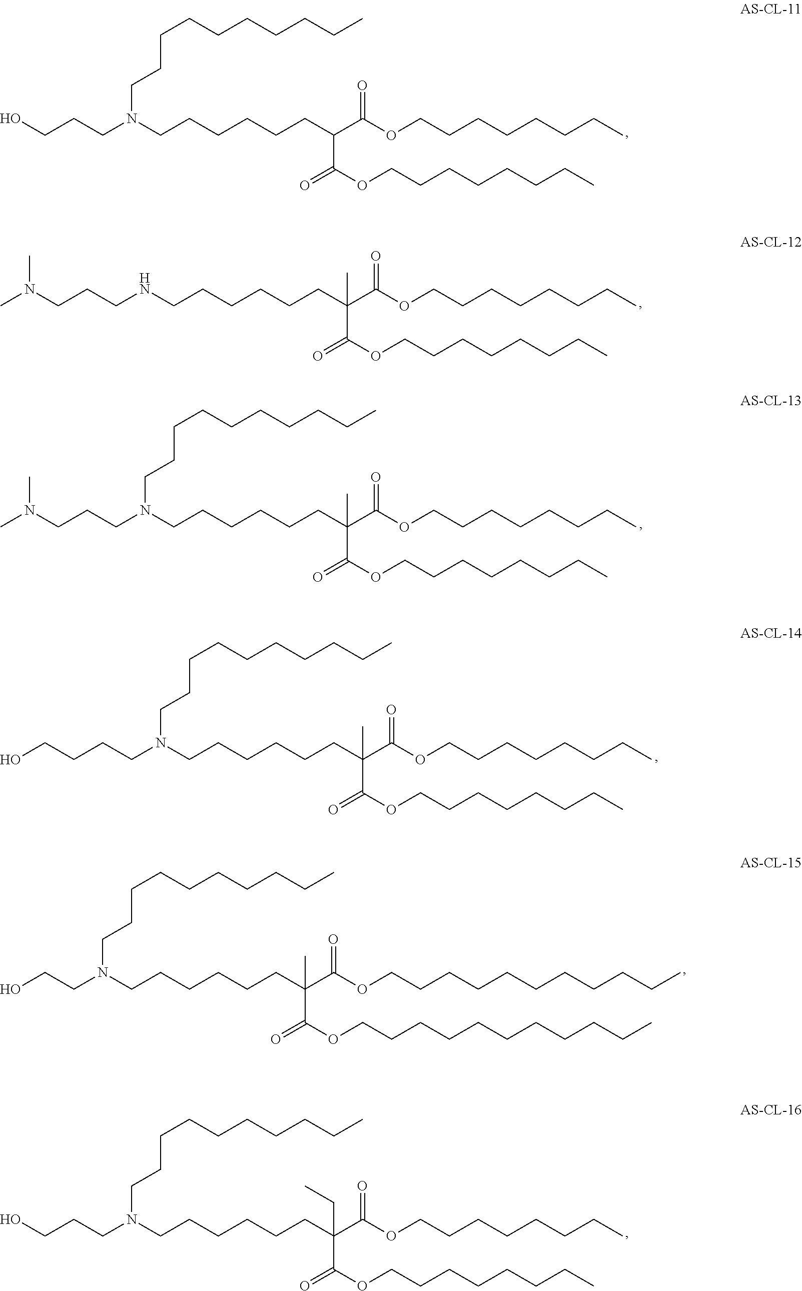

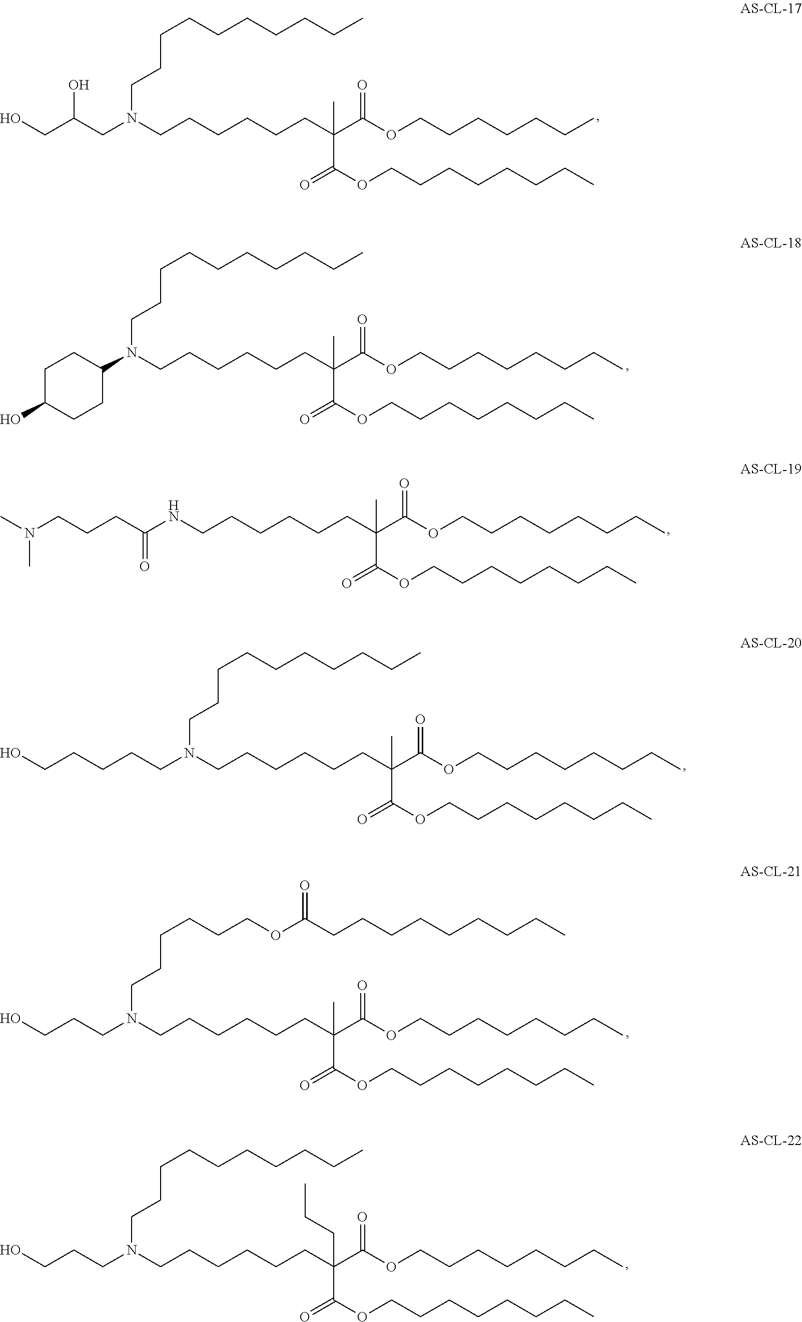

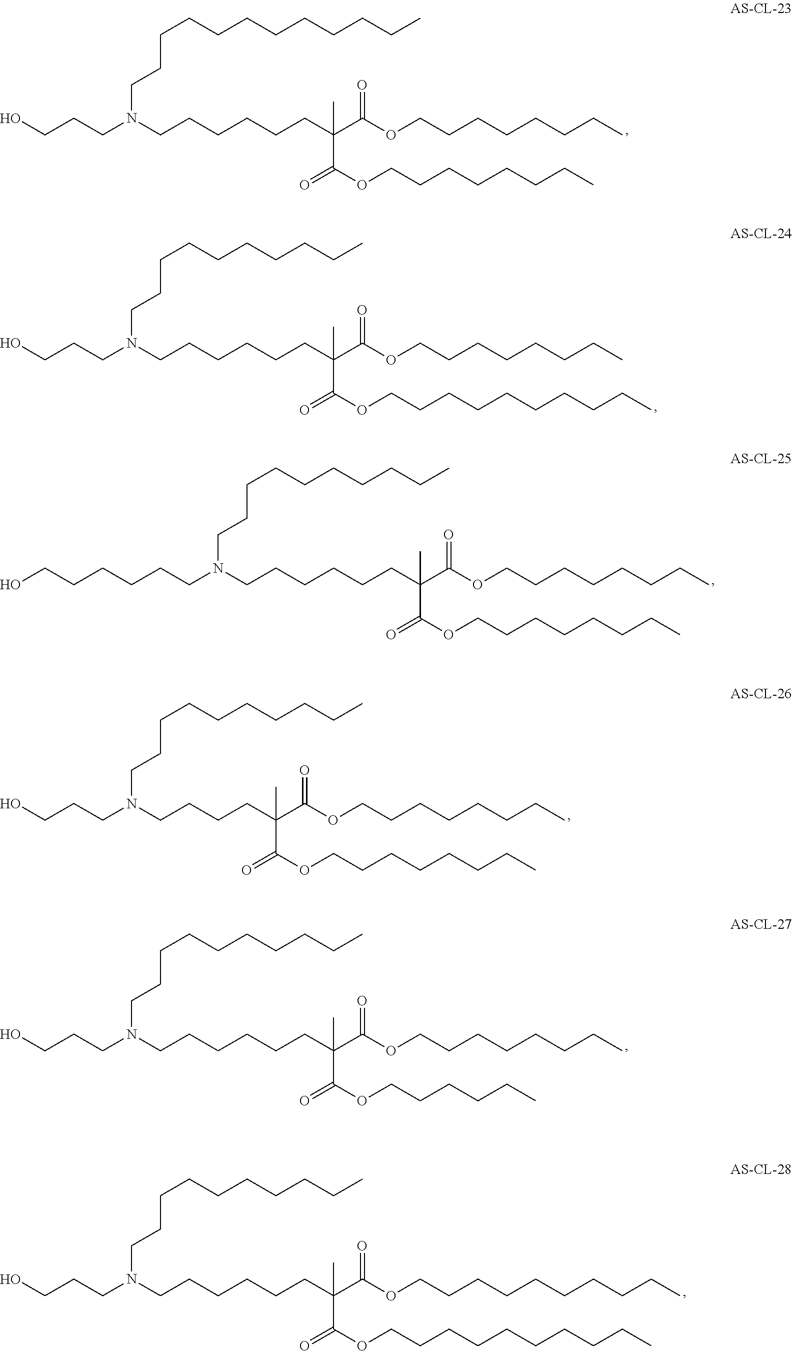 [SMILES: CCCCCCCCCCCCN(CCCO)CCCCCCC(C)(C(=O)OCCCCCCCC)C(=O)OCCCCCCCC ; CCCCCCCCCCOC(=O)C(C)(CCCCCCN(CCCO)CCCCCCCCCC)C(=O)OCCCCCCCC ; CCCCCCCCCCN(CCCCCCO)CCCCCCC(C)(C(=O)OCCCCCCCC)C(=O)OCCCCCCCC ; CCCCCCCCCCN(CCCO)CCCCC(C)(C(=O)OCCCCCCCC)C(=O)OCCCCCCCC ; CCCCCCCCCCN(CCCO)CCCCCCC(C)(C(=O)OCCCCCC)C(=O)OCCCCCCCC ; CCCCCCCCCCOC(=O)C(C)(CCCCCCN(CCCO)CCCCCCCCCC)C(=O)OCCCCCCCCCC]

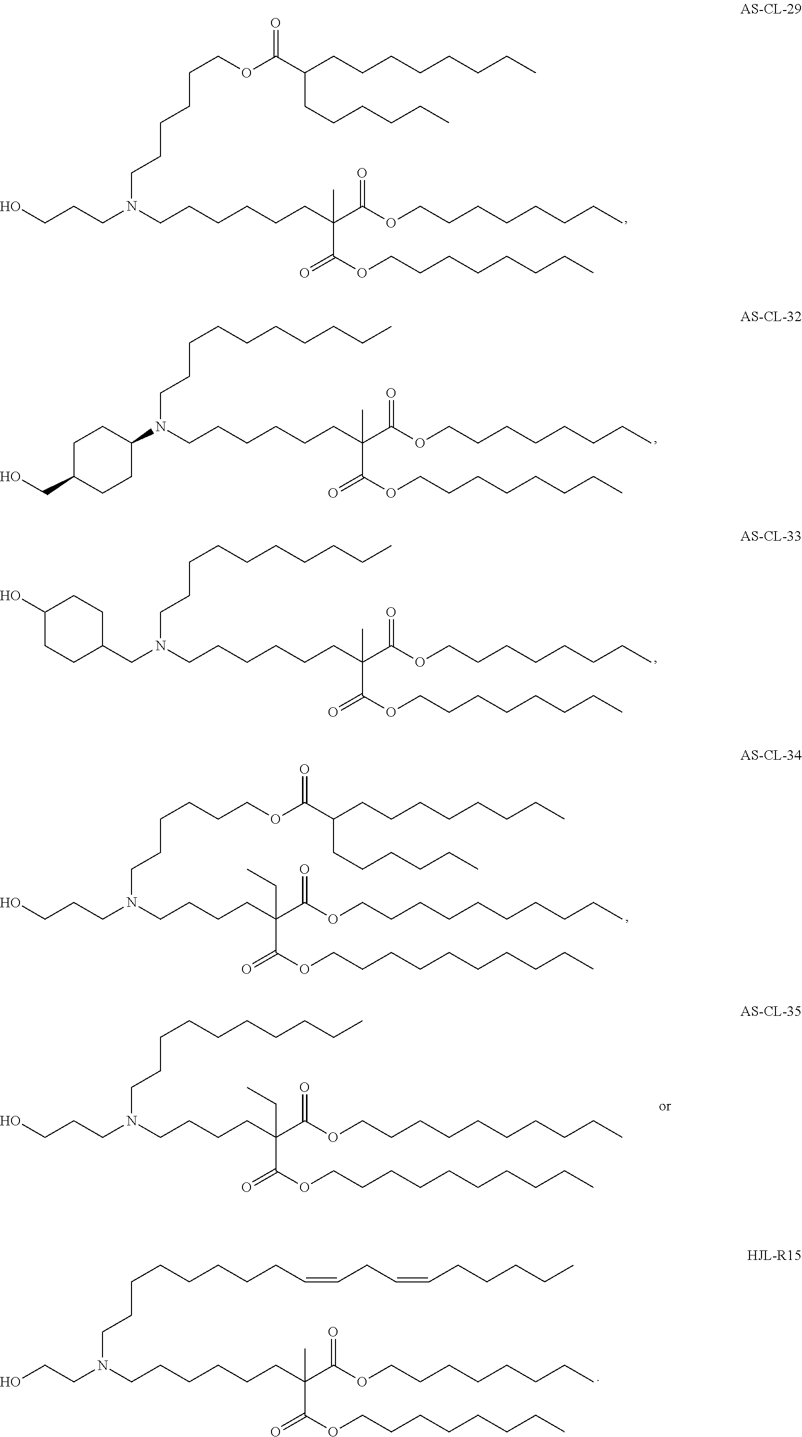 [SMILES: CCCCCCCCOC(=O)C(C)(CCCCCCN(CCCO)CCCCCCOC(=O)C(CCCCCC)CCCCCCCC)C(=O)OCCCCCCCC ; CCCCCCCCCCN(CCCCCCC(C)(C(=O)OCCCCCCCC)C(=O)OCCCCCCCC)[C@H]1CC[C@@H](CO)CC1 ; CCCCCCCCCCN(CCCCCCC(C)(C(=O)OCCCCCCCC)C(=O)OCCCCCCCC)CC1CCC(O)CC1 ; CCCCCCCCCCOC(=O)C(CC)(CCCCN(CCCO)CCCCCCOC(=O)C(CCCCCC)CCCCCCCC)C(=O)OCCCCCCCCCC ; CCCCCCCCCCOC(=O)C(CC)(CCCCN(CCCO)CCCCCCCCCC)C(=O)OCCCCCCCCCC ; CCCCC/C=C\C/C=C\CCCCCCCCN(CCO)CCCCCCC(C)(C(=O)OCCCCCCCC)C(=O)OCCCCCCCC]

According to one preferred embodiment of the present disclosure, the lipid of formula (I) has the structure of

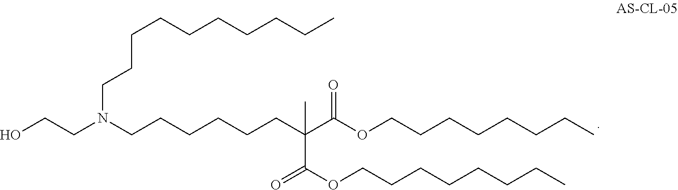 [SMILES: CCCCCCCCCCN(CCO)CCCCCCC(C)(C(=O)OCCCCCCCC)C(=O)OCCCCCCCC]

According to another preferred embodiment of the present disclosure, the lipid of formula (I) has the structure of

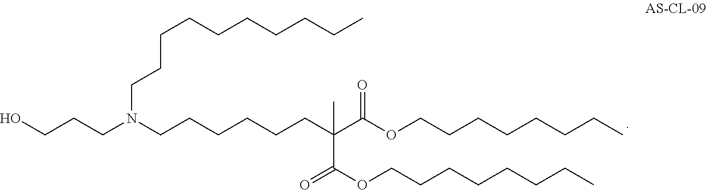 [SMILES: CCCCCCCCCCN(CCCO)CCCCCCC(C)(C(=O)OCCCCCCCC)C(=O)OCCCCCCCC]

The compounds as described herein can have the structure of formula (II), which is,

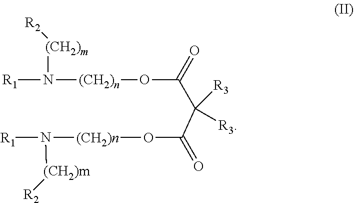

In formula (II), m and n are independently an integral between 1 to 10; and R₁ and R₃ are independently alkyl optionally substituted with one or more hydroxyl group. In some embodiments, m and 6 are independently 6, and R₁ is propyl substituted with one hydroxy group, while R₃ is —C₆H₁₃. In other embodiments, m is 10, n is 6, and R₁ is propyl substituted with one hydroxy group, while R₃ is —C₆H₁₃.

Alternatively or additionally. R₂ is H or —O(C═O)R′, in which R′ is alkyl. In some embodiments, R₂ is H. In other embodiments, R₂ is —O(C═O)R′, in which W is —C₆H₁₃.

According to embodiments of the present disclosure, the lipid of formula (II) may be any one of,

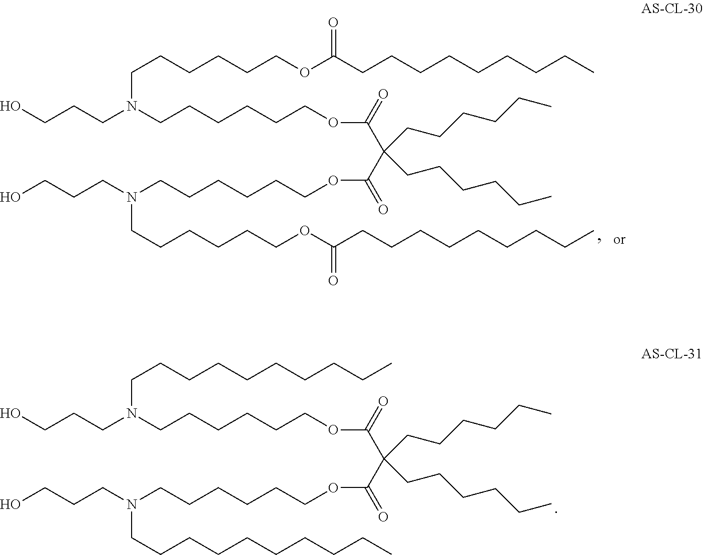 [SMILES: CCCCCCCCCC(=O)OCCCCCCN(CCCO)CCCCCCOC(=O)C(CCCCCC)(CCCCCC)C(=O)OCCCCCCN(CCCO)CCCCCCOC(=O)CCCCCCCCC ; CCCCCCCCCCN(CCCO)CCCCCCOC(=O)C(CCCCCC)(CCCCCC)C(=O)OCCCCCCN(CCCO)CCCCCCCCCC]

The compounds as described herein can have the structure of formula (III), which is described herein,

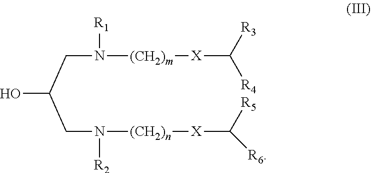

In formula (III), m is an integral between 1 to 10. Alternatively or additionally, X is —(C═O)O—; and R₁, R₂, R₃ and R₄ are independently H, or alkyl.

According to embodiments of the present disclosure, wherein the lipid of formula (III) has the structure of,

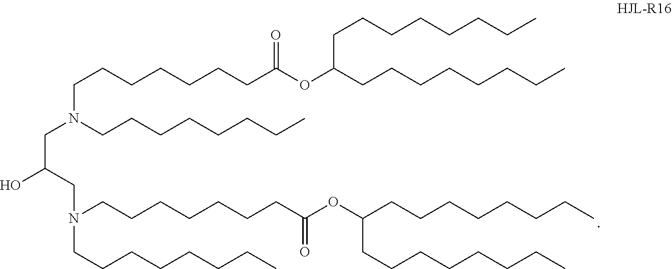 [SMILES: CCCCCCCCC(CCCCCCCC)OC(=O)CCCCCCCN(CCCCCCCC)CC(O)CN(CCCCCCCC)CCCCCCCC(=O)OC(CCCCCCCC)CCCCCCCC]

### 2. Lipid Nanoparticles

In further aspect, there is provided a lipid nanoparticle formed by one or more of lipids of the present disclosure for the delivery of an active ingredient of interest (e.g., nucleic acids of a target protein or a therapeutic agent). The lipid nanoparticle comprises in its structure, a hydrophilic core; and an outer lipid bilayer shell formed by one or more of a lipid of formula (I) to (III).

Additionally or optionally, the lipid nanoparticle can further includes a therapeutic agent disposed in the outer lipid bilayer shell or in the hydrophilic core. The therapeutic agent will be in the hydrophilic core if hydrophilic or in the lipid shell if hydrophobic. The therapeutic agent may be a nucleic acid of a target protein.

Examples of the nucleic acid that may be encapsulated within the hydrophilic core of the present lipid nanoparticle include, but are not limited to, a double strand DNA (dsDNA), a single strand DNA (ssDNA), a small interference RNA (siRNA), a short hairpin RNA (shRNA), a messenger RNA (mRNA), a micro RNA (miRNA), a transfer RNA (tRNA) and a combination thereof. In some embodiments, the lipid nanoparticle of the present disclosure further includes mRNA of a severe acute respiratory syndrome coronavirus 2 (SARS-CoV-2) encapsulated in the hydrophilic core. In further embodiments, the lipid nanoparticle of the present disclosure further includes mRNA of an envelope (E) protein of a dengue virus encapsulated in the hydrophilic core.

### 3. Use of the Lipid Nanoparticles

### 3.1 Intracellularly Delivery of Nucleic Acids

The present disclosure also provides methods for intracellularly delivery of an agent of interest (e.g., a therapeutic agent) to a cell. According to some embodiments of the present disclosure, the present lipid nanoparticle is preloaded with nucleic acids of interest (e.g., mRNA of spike protein of SARS-CoV-2) intended to be delivered to a target cell, and one or more agents to facilitate contact with, and subsequent transfection of the target cell. Preferably, the present lipid nanoparticle allows the encapsulated nucleic acids to reach the target cells, and then transfect the target cells. Thus, after delivery, the nucleic acids will encode one or more of target proteins in the target cells. The present lipid nanoparticle and methods may be used to target a vat number types of cells that include, but are not limited to, hepatocytes, epithelial cells, hematopoietic cells, endothelial cells, lung cells, bone cells, stem cells, mesenchymal stem cells, neural cells, cardiac cells, adipocytes, vascular smooth muscle cells, skeletal muscle cells, B cell, T cells, leukocytes, granulocytes, fibroblasts, reticulocytes, and etc. According to embodiments of the present disclosure, the lipid nanoparticle pre-loaded with nucleic acids of interest are successfully taken up by T cells, and subsequently transfect the T cells to express the protein of interest (e.g., the spike protein of SARS-CoV-2) encoded by the nucleic acids of interest (e.g., mRNA of the spike protein of SARS-CoV-2) delivered thereto. In certain embodiments, the protein of interest is produced at levels higher than that of a control (i.e., the baseline level of cells not being treated with the present lipid nanoparticle). According to embodiments of the present disclosure, the protein of interest is expressed by the target cells (e.g., T cells) at least 1 to 100,000-folds greater than that of the control, such as 1, 2, 3, 4, 5, 6, 7, 8, 9, 10, 20, 30, 40, 50, 55, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, and 100,000-folds greater than that of the control; preferably, at least 5 to 50,000-folds greater than that of the control, such as 5, 6, 7, 8, 9, 10, 20, 30, 40, 50, 55, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000-folds greater than that of the control; more preferably, at least 10 to 10,000-folds greater than that of the control, such as 10, 20, 30, 40, 50, 55, 60, 70, 80, 90, 100, 200, 300, 400, 500, 600, 700, 800, 900, 1,000, 2,000, 3,000, 4,000, 5,000, 6,000, 7,000, 8,000, 9,000, 10,000-folds greater than that of the control. In some embodiments, the expressed level of protein of interest remains detectable for a sustained period of time, such as 1 day, 2 days, 3 days, 4 days, 5 days, 1 week or more.

Examples of the nucleic acid that may be encapsulated within the hydrophilic core of the present lipid nanoparticle include, but are not limited to, a double strand DNA (dsDNA), a single strand DNA (ssDNA), a small interference RNA (siRNA), a short hairpin RNA (shRNA), a messenger RNA (mRNA), a micro RNA (miRNA), a transfer RNA (tRNA) and a combination thereof. In some embodiments, the lipid nanoparticle of the present disclosure further includes mRNA of a severe acute respiratory syndrome coronavirus 2 (SARS-CoV-2) encapsulated in the hydrophilic core. In further embodiments, the lipid nanoparticle of the present disclosure further includes mRNA of an envelope (E) protein of a dengue virus encapsulated in the hydrophilic core.

### 3.2 Treatment of Diseases

The present disclosure also provides methods for the treatment and/or prophylaxis of a disease in a subject. The method includes administering to a target tissue of the subject an effective amount of the present lipid nanoparticle pre-loaded with a therapeutic agent therein, so as to treat and/or prevent the disease.

As used herein, the term “subject” refers to any animal including, but not limited to, humans, non-human primates, rodents, and the like, to which the lipid nanoparticles of the present disclosure pre-loaded with a therapeutic agent are administered. Typically, the term “subject” as used herein refers to a human subject. The therapeutic agent may be an nucleic acids of a target protein, and the like.

In some embodiments, the method includes administering to the subject the lipid nanoparticles of the present disclosure pre-loaded with viral nucleic acids, thus the encapsulated viral nucleic acids of interest are delivered to the target tissue (e.g., lung, liver, and etc) of the subject, and are expressed in the target tissue and act as antigens to elicit a controlled level of an immune response in the subject to immune the subject, thereby preventing the subject from being subsequently infected by said virus and/or from developing diseases caused by said viral infection (e.g., severe acute respiratory syndrome, SARS).

Additionally or optionally, the lipid nanoparticles of the present disclosure is formulated in combination with one or more additional carriers, excipients, or stabilizing agents. The lipid nanoparticles of the present disclosure may be administered and dosed in accordance with current medical practice, taking into account of the clinical condition of the subject, the site and method of administration, the scheduling of administration, the subject's age, sex, body weight and other factors relevant to the clinical condition. The effective amount for the purpose herein may be determined by such relevant factors known to those of ordinary skill in clinical research, pharmacological, clinical and medical arts. In some embodiments, the amount administered is effective to achieve at least some level of stabilizing, improving, or eliminating symptoms of the disease, or prevents the disease from progressing. For example, a suitable amount and dosing regimen is one that causes at least transient protein production.

Suitable routes of administration include, for example, oral, rectal, vaginal, transmucosal, pulmonary (e.g., intratracheal, inhaled, and etc), or intestinal administration; parenteral delivery including intramuscular, subcutaneous, intramedullary injection, as well as intrathecal, direct intraventricular, intravenous, intraperitoneal, intranasal or intraocular injection.

Alternatively, the lipid nanoparticles of the present disclosure may be administered in a local rather systemic manner, for example, via injection of the lipid nanoparticles directly into a target tissue. Local delivery may be affected in various ways, depending on the tissue to be targeted. For example, aerosols containing the present lipid nanoparticles can be inhaled (for nasal, tracheal or bronchial delivery); lipid nanoparticles of the present disclosure can be injected into the site where the disease manifests, or where pain occurs. Also, the lipid nanoparticles may be provided in lozenges for oral, tracheal, or esophageal application; or can be supplied in liquid, tablet or capsule form for administration to the stomach or intestines; or can be delivered to the eye by use of creams, drops, or even injection.

In some embodiments, the lipid nanoparticles of the present disclosure are formulated such that it is suitable for extended-release of nucleic acids contained therein. Such extended-release lipid nanoparticles may be administered to the subject at extended dosing intervals. For example, the lipid nanoparticles of the present disclosure may be administered to a subject daily, or twice per day, or every other day. In some preferred embodiments, the lipid nanoparticles are administered to the subject once a week, twice a week, every 10 days, every two weeks, every three weeks, or every four weeks, once a month, every six weeks, every eight weeks, every other month, every other month, every three months, every four months, every six months, every eight months, every nine months or annually. Also contemplated herein are compositions which are formulated for depot administration (e.g., by intramuscularly, subcutaneously, and etc) to either deliver or release nucleic acids over extended period of time.

Also contemplated herein are lyophilized compositions comprising one or more of the lipid nanoparticles disclosed herein. The lyophilized composition of the present disclosure may be reconstituted prior to administration or can be reconstituted in vivo. For example, a lyophilized composition can be formulated in an appropriate dosage form (e.g., an intradermal dosage such as a disk, rod, or membrane) and administered such that the dosage form is rehydrated over time in vivo by the individual's body fluid.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

### Evaluation of In Vitro SARS-CoV-2 S Protein Expression by Cellular E11

SARS-CoV-2 S protein mRNA-LNPs were individually transfected into 293T cells and cultured at 37° C. in DMEM medium containing 10% FBS for 24 h. After transfection, the cells were fixed with 4% paraformaldehyde/PBS for 15 min, and then were permeabilized with 0.1% Triton X-100 for 10 min. After washing, 100 ng/ml anti-RBD chimeric antibodies were added to the wells for 1 hr at room temperature. Then, the horseradish peroxidase-conjugated anti-human antibody (1:8000) were added for 1 hr at room temperature, as appropriate. The plates were washed three times with PBS containing 0.1% Tween-20 (PBST0.1) and then incubated for 1 hour with peroxidase-affinipure goat anti-mouse IgG (H+L) (Jackson ImmunoResearch) (1:5000 dilution). After three washes with PBST0.1, the signal was produced using 3,3′5,5′-Tetramethylbenzidine (TMB) color development (TMBW-1000-01, SURMODICS). Finally, the reaction was stopped with 3 N HCl, and absorbance was measured at 450 nm by an ELISA reader (Versa Max Tunable Microplate Reader; Molecular Devices).

### Mouse Immunization

Groups of 6- to 8-week-old BALB/c mice were immunized via intramuscular (i.m.) injection with designated mRNP-LNP (i.e., MC3-LNP, SM102-LNP, AS-CL05-LNP, AS-CL09-LNP, AS-CL28-LNP, or AS-CL35-LNP each dose contained 10 μg of mRNA) at the beginning of the experiment (day 0), followed by additional boost injections respectively at the second and 4 weeks. Serum samples were collected 4, 6, and 8 weeks after the first immunization (day 0) and tested for binding activity towards FLS-WT or FLS-BA.5 protein, and neutralization activity via pseudovirus assay.

### Pseudovirus Neutralization Assay

Blood samples were collected from mice 6 weeks after the first boost, and the sera were used to determine the neutralization activity against BA.5 SARS-CoV-2 pseudoviruses. The pseudovirus neutralization assays were performed using SARS-CoV-2 pseudotyped lentiviruses expressing full-length S protein and firefly luciferase in HEK293T cells overexpressing human ACE2 (HEK293T/hACE2; purchased from the National RNAi Core Facility, Academia Sinica, Taiwan). The half-maximal inhibitory concentration (IC₅₀) was calculated by nonlinear regression using Prism software version 8.1.0 (GraphPad Software Inc.). The average IC₅₀ value for each experimental group was determined from three independent experiments.

### Plaque Reduction Neutralization Titer (PRNT) Assay

Sera from animals injected with DENV2 E mRNA-LNPs were serially diluted in PBS and pre-incubated with 100 plaque-forming units (PFU) DENV2 for 1 h at 37° C. The mixtures were then added to pre-seeded BHK-21 cells for 1 h at 37° C. The virus-containing culture medium was removed and replaced with DMEM containing 2% FBS and 1% methyl-cellulose for an additional 4-day incubation. The cells were fixed with 10% formaldehyde overnight and stained with 0.5% crystal violet for 20 min. The plates were then washed with tap water, and the numbers of plaques formed at each dilution were counted. Each experiment was performed in triplicate. Plaque reduction was calculated as follows: Inhibition percentage=100×[1−(plaque number with immunized serum/plaque number without immunized serum)]. The 50% plaque reduction (PRNT₅₀) value was calculated with Prism software. The DENV2 strain 16881 was used in this study.

### Example 1 Synthesis of the Present Compounds

In general, the compounds of the present disclosure were synthesized in accordance with the procedures outlined in Schemes 1 to 5, in which the amines required for step 7 in scheme 1 are listed in Table 1.

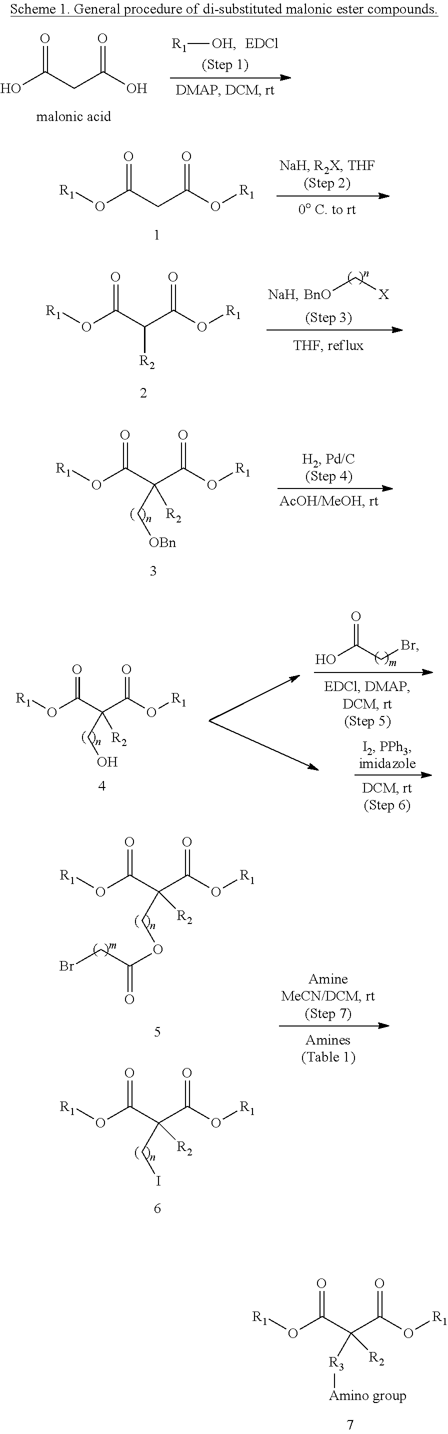

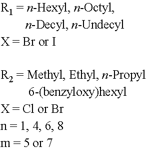

TABLE 1
The list of amines.

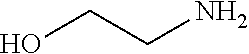

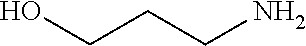

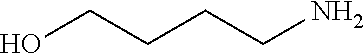

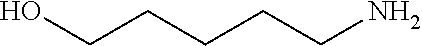

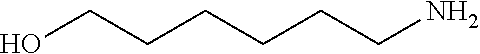

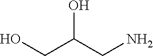

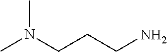

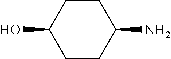

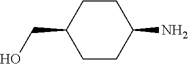

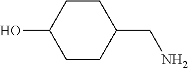

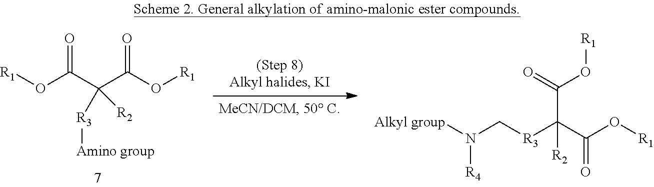

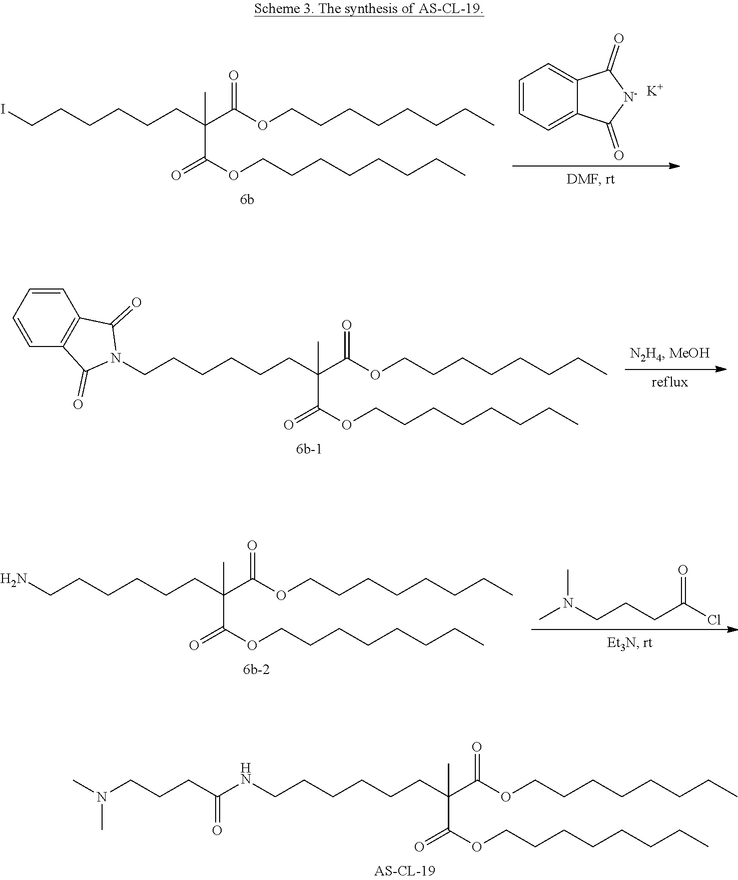

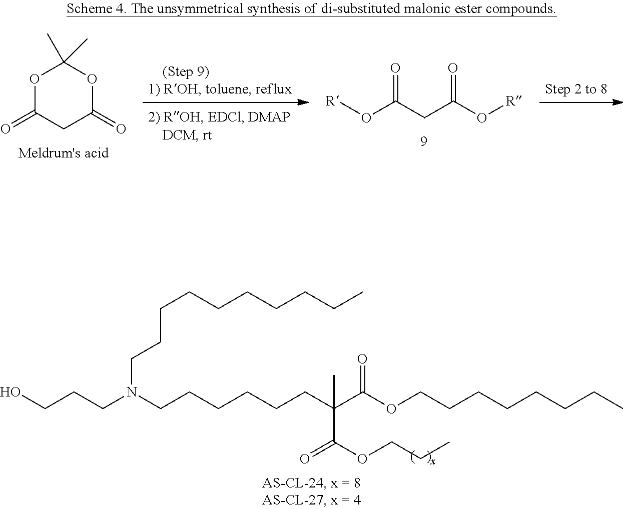

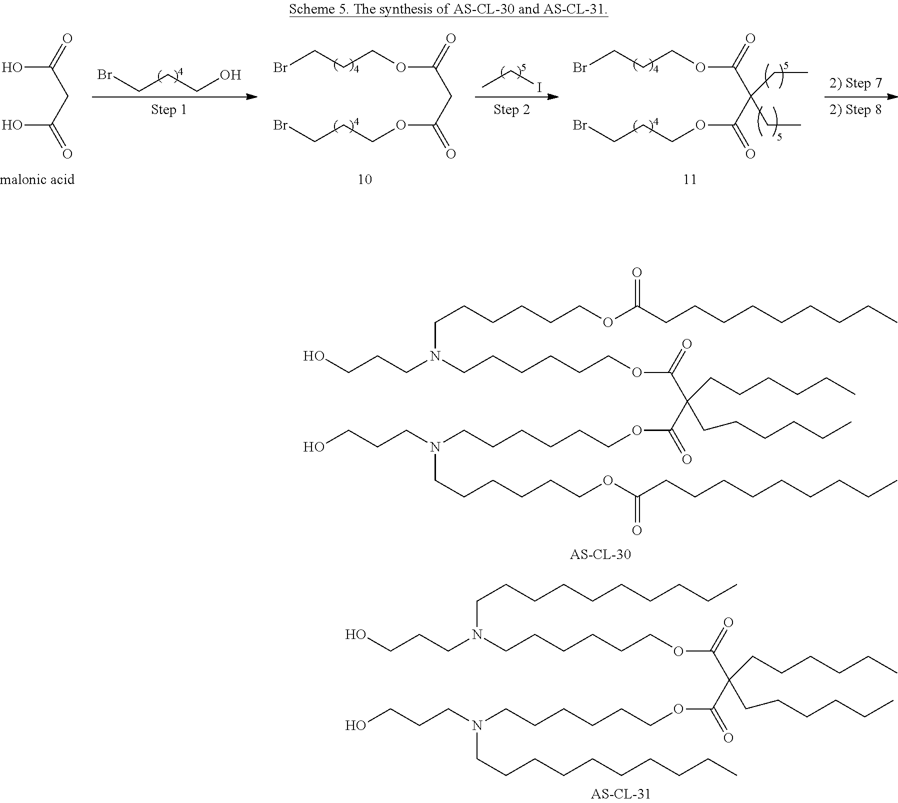

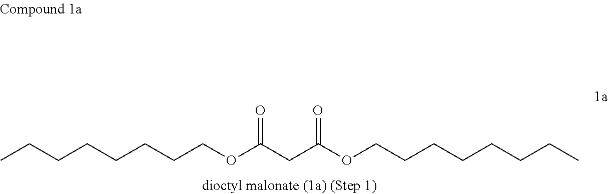 [SMILES: CCCCCCCCOC(=O)CC(=O)OCCCCCCCC]

A mixture solution of malonic acid (2.61 g, 25.08 mmol) and octanol (7.18 g, 55.13 mmol) in dicholomethane (DCM) was stirred at 0° C., and then the addition of 1-Ethyl-3-(3-dimethylaminopropyl)carbodiimide (EDCI) (10.58 g, 55.18 mmol) and (4-dimethylaminopyridine) DMAP (613 mg, 5 mmol). After stirring at R.T overnight, the reaction solution was washed 2N HCl_{(aq)} and brine and dried over MgSO₄. After evaporation, the residue was purified by silica gel column chromatography with ethyl acetate/hexane (EA/Hea) (1/20) to yield the compound 1a (7.66 g, 23.32 mmol) as a colorless oil. ¹H NMR (600 MHz, CDCl₃) δ 4.12 (t, J=6.6 Hz, 4H), 3.35 (s, 2H), 1.63-1.60 (m, 4H), 1.32-1.26 (m, 20H), 0.86 (t, J=7.2 Hz, 6H). ¹³C NMR (150 MHz, CDCl₃) δ 166.7, 65.6, 41.7, 31.7, 29.1(×2), 28.4, 25.8, 22.6, 14.0.

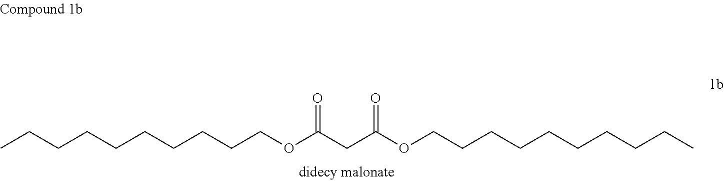 [SMILES: CCCCCCCCCCOC(=O)CC(=O)OCCCCCCCCCC]

Compound 1b was synthesized according to the general step 1 procedure in scheme 1. The title compound was obtained as a colorless oil. ¹H NMR (600 MHz, CDCl₃) δ 4.12 (t, J=6.6 Hz, 4H), 3.35 (s, 2H), 1.65-1.60 (m, 4H), 1.33-1.25 (m, 28H), 0.87 (t, J=7.2 Hz, 6H), 20 ¹³C NMR (150 MHz, CDCl₃) δ 166.6, 65.6, 41.6, 31.8, 29.4 (×2), 29.2, 29.1, 28.4, 25.7, 22.6, 14.0.

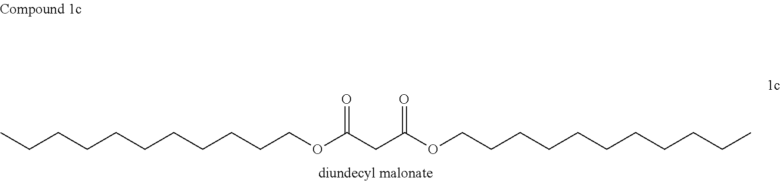 [SMILES: CCCCCCCCCCCOC(=O)CC(=O)OCCCCCCCCCCC]

Compound 1c was synthesized according to the general step 1 procedure in scheme 1. The title compound was obtained as a colorless oil. ¹H NMR (600 MHz, CDCl₃) δ 4.12 (t, J=6.6 Hz, 4H), 3.35 (s, 2H), 1.65-1.59 (m, 4H), 1.32-1.25 (m, 32H), 0.87 (t, J=7.2 Hz, 6H). ¹³C NMR (150 MHz, CDCl₃) δ 170.3, 65.6, 46.3, 32.0, 29.7, 29.6, 29.4, 29.3, 28.6, 25.9, 22.8, 14.2, 13.7.

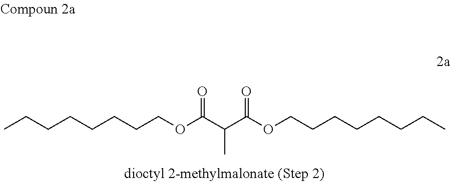 [SMILES: CCCCCCCCOC(=O)C(C)C(=O)OCCCCCCCC]

NaH (430 mg, 10.8 mmol) was added to a solution of dioctyl malonate (4.43 g, 13.5 mmol) in THF at 0° C., and then the addition of MeI (0.66 mL, 10.8 mmol). After stirring at R.T overnight, the reaction solution was washed sat. NH₄Cl_{(aq)} and brine and dried over MaSO₄. After evaporation, the residue was purified by silica gel column chromatography with EA/Hea (1/40) to yield the compound 2a (2.45 g, 7.15 mmol) as a colorless oil. ¹H NMR (600 MHz, CDCl₃) δ 4.15-4.08 (m, 4H), 3.42 (q, J=7.2 Hz, 1H), 1.65-1.57 (m, 4H), 1.41 (d, J=7.2 Hz, 3H), 1.33-1.28 (m, 20H), 0.88 (t, J=7.2 Hz, 6H). ¹³C NMR (150 MHz, CDCl₃) δ 170.2, 65.5, 46.2, 31.8, 29.2(×2), 28.5, 25.8, 22.6, 14.1, 13.6.

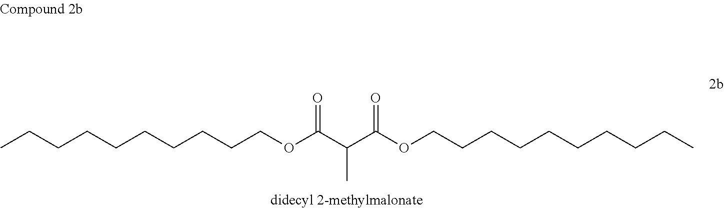 [SMILES: CCCCCCCCCCOC(=O)C(C)C(=O)OCCCCCCCCCC]

Compound 2b was synthesized according to the general step 2 procedure in scheme 1. The title compound was obtained as a colorless oil. ¹H NMR (600 MHz, CDCl₃) δ 4.14-4.09 (m, 4H), 3.42 (q, J=7.2 Hz, 1H), 1.64-1.60 (m, 4H), 1.41 (d, J=7.2 Hz, 3H), 1.32-1.25 (m, 28H), 0.87 (t, J=7.2 Hz, 6H). ¹³C NMR (150 MHz, CDCl₃) δ 170.2, 65.5, 46.2, 31.8, 29.5 (×2), 29.3, 29.2, 28.5, 25.8, 22.6, 14.1.

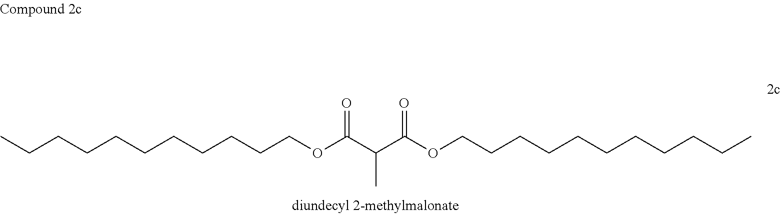 [SMILES: CCCCCCCCCCCOC(=O)C(C)C(=O)OCCCCCCCCCCC]

Compound 2c was synthesized according to the general step 2 procedure in scheme 1. The title compound was obtained as a colorless oil. ¹H NMR (600 MHz, CDCl₃) δ 4.12-4.07 (m, 4H), 3.40 (q, J=7.2 Hz, 1H), 1.61-1.59 (m, 6H), 1.39 (d, J=7.2 Hz, 3H), 1.35-1.25 (m, 30H), 0.85 (t, J=7.2 Hz, 6H). ¹³C NMR (150 MHz, CDCl₃) δ 170.3, 65.6, 46.3, 32.0, 29.7, 29.6, 29.4, 29.3, 28.6, 25.9, 22.8, 14.2, 13.7.

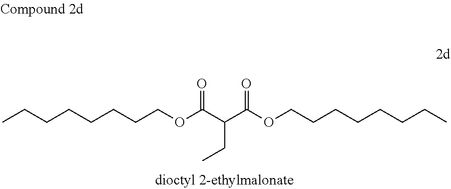 [SMILES: CCCCCCCCOC(=O)C(CC)C(=O)OCCCCCCCC]

Compound 2d was synthesized according to the general step 2 procedure in scheme 1. The title compound was obtained as a colorless oil. ¹H NMR (600 MHz, CDCl₃) δ 4.14-4.08 (m, 4H), 3.24 (t, J=7.2 Hz, 1H), 1.94-1.89 (m, 2H), 1.64-1.58 (m, 4H), 1.35-1.26 (m, 20H), 0.95 (t, J=7.2 Hz, 3H), 0.87 (t, J=7.2 Hz, 6H). ¹³C NMR (150 MHz, CDCl₃) δ 169.5, 65.4, 53.6, 31.7, 29.1, 28.5, 25.8, 22.6, 22.2, 14.0, 11.8.

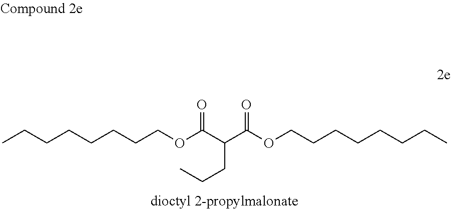 [SMILES: CCCCCCCCOC(=O)C(CCC)C(=O)OCCCCCCCC]

Compound 2e was synthesized according to the general step 2 procedure in scheme 1. The title compound was obtained as a colorless oil. ¹H NMR (600 MHz, CDCl₃) δ 4.15-4.08 (m, 4H), 3.33 (t, J=7.8 Hz, 1H), 1.89-1.85 (m, 2H), 1.64-1.60 (m, 4H), 1.37-1.27 (m, 22H), 0.93 (t, J=7.2 Hz, 6H), 0.88 (t, J=7.2 Hz, 6H). ¹³C NMR (150 MHz, CDCl₃) δ 169.7, 65.4, 51.9, 31.7, 30.8, 29.1, 28.5, 25.8, 22.6, 20.6 14.0, 13.7. MS (ESI): m/z [M+Na]⁺ 393.2973 for C₂₂H₄₂O₄Na.

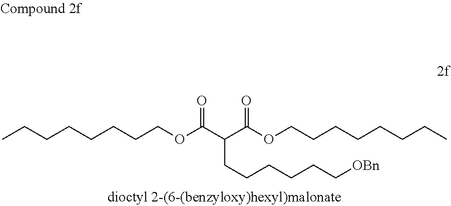 [SMILES: CCCCCCCCOC(=O)C(CCCCCCOCc1ccccc1)C(=O)OCCCCCCCC]

Compound 2f was synthesized according to the general step 2 procedure in scheme 1. The title compound was obtained as a colorless oil. ¹H NMR (600 MHz, CDCl₃) δ 7.35-7.26 (m, 5H), 4.49 (s, 2H), 4.15-4.08 (m, 4H), 3.45 (t, J=6.6 Hz, 2H), 3.31 (t, J=6.6 Hz, 1H), 1.90-1.86 (m, 2H), 1.64-1.59 (m, 6H), 1.33-1.29 (m, 26H), 0.86 (t, J=7.2 Hz, 6H). ¹³C NMR (150 MHz, CDCl₃) δ 169.6, 138.5, 128.3, 127.6, 127.5, 72.9, 70.3, 65.4, 52.1, 31.8, 29.6, 29.2 (×2), 29.1, 28.7, 28.5, 27.3, 25.9, 25.8, 22.6, 14.1.

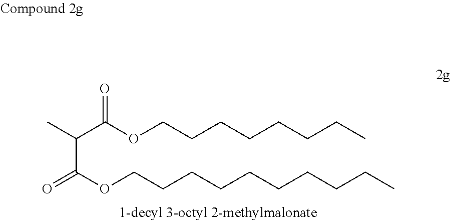 [SMILES: CCCCCCCCCCOC(=O)C(C)C(=O)OCCCCCCCC]

Compound 2g was synthesized according to the general step 2 procedure in scheme 1. The title compound was obtained as a colorless oil. ¹H NMR (600 MHz, CDCl₃) δ 4.15-4.08 (m, 4H), 3.42 (q, J=7.2 Hz, 1H), 1.65-1.60 (m, 4H), 1.41 (d, J=7.2 Hz, 3H), 1.33-1.26 (m, 24H), 0.89-0.87 (m, 6H). ¹³C NMR (150 MHz, CDCl₃) δ 170.2, 65.5, 64.2, 31.9, 31.8, 29.6, 29.3, 29.25, 29.21, 28.5, 25.8, 22.7, 22.6, 14.10, 14.08, 13.6.

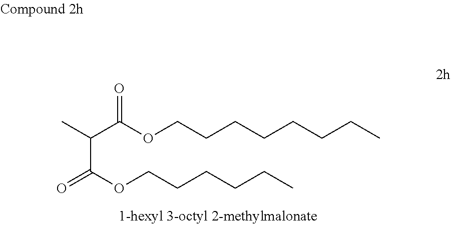 [SMILES: CCCCCCCCOC(=O)C(C)C(=O)OCCCCCC]

Compound 2h was synthesized according to the general step 2 procedure in scheme 1. The title compound was obtained as a colorless oil. ¹H NMR (600 MHz, CDCl3) δ 4.15-4.08 (m, 4H), 3.42 (q, J=7.2 Hz, 1H), 1.65-1.60 (m, 4H), 1.41 (d, J=7.2 Hz, 3H), 1.30-1.27 (m, 16H), 0.89-0.87 (m, 6H). ¹³C NMR (150 MHz, CDCl3) δ 170.3, 65.5, 46.2, 31.8, 31.4, 29.2 (×2), 28.5, 28.4, 25.8, 25.5, 22.6, 22.5, 14.1, 14.0, 13.6.

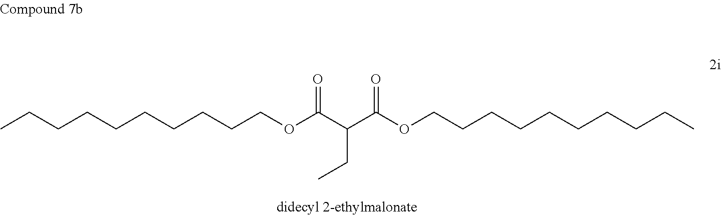 [SMILES: CCCCCCCCCCOC(=O)C(CC)C(=O)OCCCCCCCCCC]

Compound 2i was synthesized according to the general step 2 procedure in scheme 1. The title compound was obtained as a colorless oil. ¹H NMR (600 MHz, CDCl₃) δ 4.14-4.09 (m, 4H), 3.25 (t, J=7.2 Hz, 1H), 1.95-1.90 (m, 2H), 1.64-1.60 (m, 4H), 1.32-1.25 (m, 26H), 0.96 (t, J=7.2 Hz, 3H), 0.87 (t, J=7.2 Hz, 6H). ¹³C NMR (150 MHz, CDCl₃) δ 169.7, 65.5, 53.7, 32.0, 29.6 (×2), 29.4, 29.3, 28.6, 25.9, 22.8, 22.3, 14.2, 12.0.

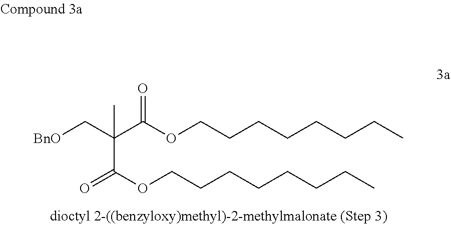 [SMILES: CCCCCCCCOC(=O)C(C)(COCc1ccccc1)C(=O)OCCCCCCCC]

NaH (165 mg, 4.13 mmol) was added to a solution of dioctyl 2-methylmalonate (1.4 g, 4.08 mmol) in THF at 0° C., and then the addition of benzyl chloromethyl ether (0.56 mL, 4.08 mmol). After stirring under reflux overnight, the reaction solution was washed with sat. NH₄Cl_{(aq)} and brine and dried over MaSO₄. After evaporation, the residue was purified by silica gel column chromatography with EA/Hex (1/25) to yield the compound 3a (1.4 g, 3.02 mmol, 74%) as a colorless oil. ¹H NMR (600 MHz, CDCl₃) δ 7.36-7.27 (m, 5H), 4.53 (s, 2H), 4.10 (t, J=6.6 Hz, 4H), 3.81 (s, 2H), 1.60-1.57 (m, 4H), 1.53 (s, 3H), 1.30-1.25 (m, 20H), 0.88 (t, J=7.2 Hz, 6H). ¹³C NMR (150 MHz, CDCl₃) δ 170.7, 138.0, 128.3, 127.5, 127.4, 73.4, 72.7, 65.5, 54.9, 31.8, 29.2(×2), 28.5, 25.8, 22.6, 18.5, 14.1.

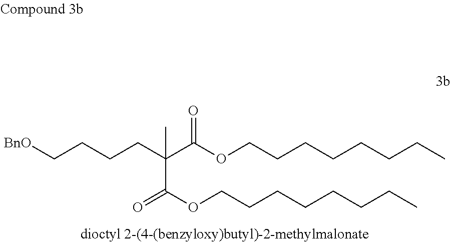 [SMILES: CCCCCCCCOC(=O)C(C)(CCCCOCc1ccccc1)C(=O)OCCCCCCCC]

Compound 3b was synthesized according to the general step 3 procedure in scheme 1. The title compound was obtained as a colorless oil. ¹H NMR (600 MHz, CDCl₃) δ 7.35-7.27 ((m, 5H), 4.48 (s, 2H), 4.09 (t, J=6.6 Hz, 4H), 3.46 (t, J=6.6 Hz, 2H) 1.88-1.86 (m, 2H), 1.65-1.58 (m, 6H), 1.40 (s, 3H), 1.29-1.26 (m, 22H), 0.88 (t, J=7.2 Hz, 6H); ¹³C NMR (150 MHz, CDCl₃) δ 172.4, 138.5, 128.3, 127.5, 127.4, 72.8, 69.9, 65.3, 53.7, 35.3, 31.7, 29.9, 29.1 (×2), 28.4, 25.8, 22.6, 21.0, 19.8, 14.0; MS (ESI): m/z [M+Na]⁺ 527.3703 for C₃₁H₅₁O₅Na.

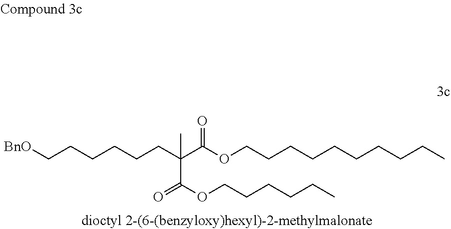 [SMILES: CCCCCCCCCCOC(=O)C(C)(CCCCCCOCc1ccccc1)C(=O)OCCCCCC]

Compound 3c was synthesized according to the general step 3 procedure in scheme 1. The title compound was obtained as a light-yellow oil. ¹H NMR (600 MHz, CDCl₃) δ 7.35-7.27 (m, 5H), 4.49 (s, 2H), 4.09 (t, J=6.6 Hz, 4H), 3.45 (t, J=6.6 Hz, 2H), 1.85-1.83 (m, 2H), 1.62-1.57 (m, 4H), 1.39 (s, 3H), 1.38-1.24 (m, 28H), 0.88 (t, J=7.2 Hz, 6H). ¹³C NMR (150 MHz, CDCl₃) δ 172.6, 138.7, 128.3, 127.6, 127.5, 72.9, 70.4, 65.3, 53.8, 35.5, 31.2, 29.7, 29.6 (×2), 29.2, 28.5, 26.0, 25.8, 24.3, 22.6, 19.9, 14.1.

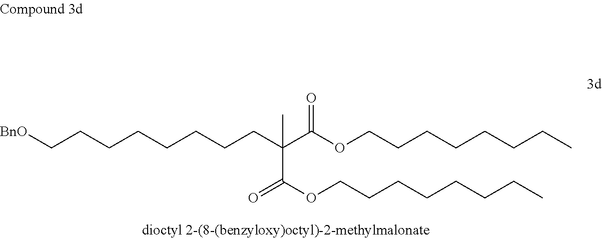 [SMILES: CCCCCCCCOC(=O)C(C)(CCCCCCCCOCc1ccccc1)C(=O)OCCCCCCCC]

Compound 3d was synthesized according to the general step 3 procedure in scheme 1. The title compound was obtained as a light-yellow oil. ¹H NMR (600 MHz, CDCl₃) δ 7.35-7.26 (m, 5H), 4.50 (s, 2H), 4.13-4.06 (m, 4H), 3.45 (t, J=6.6 Hz, 2H), 1.85-1.82 (m, 2H), 1.62-1.55 (m, 6H), 1.39 (s, 3H), 1.33-1.26 (m, 30H), 0.88 (t, J=7.2 Hz, 6H). ¹³C NMR (150 MHz, CDCl₃) δ 172.6, 138.7, 128.3, 127.6, 127.4, 72.8, 70.5, 65.2, 53.8, 35.6, 31.8, 29.9, 29.7 (×2), 29.4, 29.3, 29.2, 28.5, 26.2, 25.9, 24.3, 22.6, 19.9, 14.1.

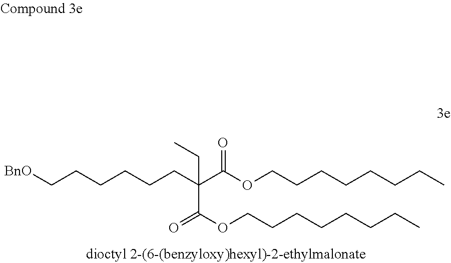 [SMILES: CCCCCCCCOC(=O)C(CC)(CCCCCCOCc1ccccc1)C(=O)OCCCCCCCC]

Compound 3e was synthesized according to the general step 3 procedure in scheme 1. The title compound was obtained as a light-yellow oil. ¹H NMR (600 MHz, CDCl₃) δ 7.35-7.26 (m, 5H), 4.49 (s, 2H), 4.09 (t, J=6.6 Hz, 4H), 3.45 (t, J=6.6 Hz, 2H), 1.92 (q, J=7.2 Hz, 2H), 1.87-1.84 (m, 2H), 1.61-1.58 (m, 6H), 1.38-1.26 (m, 24H), 1.16-1.12 (m, 2H), 0.88 (t, J=6.6 Hz, 6H), 0.80 (t, J=6.6 Hz, 3H). ¹³C NMR (150 MHz, CDCl₃) δ 171.9, 138.5, 128.2, 127.5, 127.3, 72.7, 70.2, 65.0, 57.9, 31.7, 31.5, 29.6, 29.5, 29.1, 29.0, 28.4, 25.9, 25.8, 25.1, 23.8, 22.5, 14.0, 8.3.

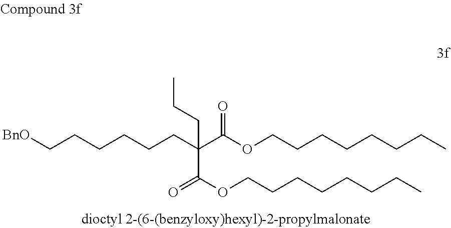 [SMILES: CCCCCCCCOC(=O)C(CCC)(CCCCCCOCc1ccccc1)C(=O)OCCCCCCCC]

Compound 3f was synthesized according to the general step 3 procedure in scheme 1. The title compound was obtained as a light-yellow oil. ¹H NMR (600 MHz, CDCl₃) δ 7.35-7.27 (m, 5H), 4.50 (s, 2H), 4.08 (t, J=6.6 Hz, 4H), 3.47-3.43 (m, 2H) 1.87-1.82 (m, 4H), 1.61-1.58 (m, 6H), 1.36-1.26 (m, 26H), 1.17-1.15 (m, 2H), 0.91 (t, J=7.2 Hz, 3H), 0.88 (t, J=7.2 Hz, 6H). ¹³C NMR (150 MHz, CDCl₃) δ 172.0, 138.6, 128.3, 127.5, 127.4, 72.8, 70.3, 65.1, 57.6, 35.5, 32.2, 31.8, 29.7, 29.6, 29.2, 29.1, 28.5, 25.9, 25.8, 24.0, 22.6, 17.4, 14.4, 14.0.

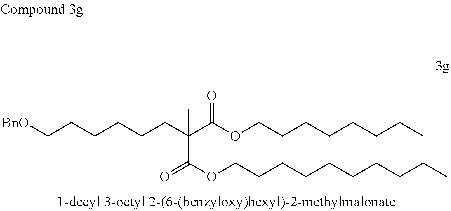 [SMILES: CCCCCCCCCCOC(=O)C(C)(CCCCCCOCc1ccccc1)C(=O)OCCCCCCCC]

Compound 3g was synthesized according to the general step 3 procedure in scheme 1. The title compound was obtained as a colorless oil. ¹H NMR (60) MHz, CDCl3) δ 7.34-7.25 (m, 5H), 4.49 (s, 2H), 4.10-4.08 (m, 4H), 3.45 (t, J=6.6 Hz, 2H), 1.86-1.83 (m, 2H), 1.62-1.58 (m, 6H), 1.39 (s, 3H), 1.38-1.19 (m, 30H), 0.89-0.87 ((m, 6H). ¹³C NMR (150 MHz, CDCl₃) δ 172.6, 138.7, 128.3, 127.6, 127.5, 72.9, 70.4, 65.3, 53.8, 35.6, 31.9, 31.8, 29.8, 29.7, 229.6, 29.3, 29.24, 29.22, 29.20, 28.5, 26.0, 25.9, 24.3, 22.7, 22.6, 19.9, 14.11, 14.09. MS (ESI): m/z [M+Na]⁺ 583.4333 for C₃₅H₆₀O₅Na.

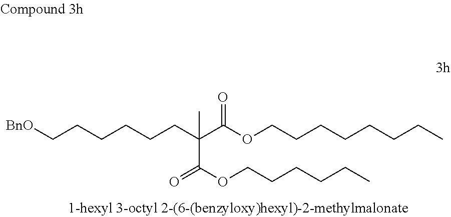 [SMILES: CCCCCCCCOC(=O)C(C)(CCCCCCOCc1ccccc1)C(=O)OCCCCCC]

Compound 3h was synthesized according to the general step 3 procedure in scheme 1. The title compound was obtained as a colorless oil. ¹H NMR (600 MHz, CDCl₃) δ 7.35-7.27 ((m, 5H), 4.49 (s, 2H), 4.09 (t, J=6.6 Hz, 4H), 3.45 (t, J=6.6 Hz, 2H) 1.85-1.83 (m, 2H), 1.62-1.59 (m, 6H), 1.38 (s, 3H), 1.32-1.26 (m, 22H), 0.89-0.87 (m, 6H). ¹³C NMR (150 MHz, CDCl₃) δ 172.5, 138.6, 128.3, 127.6, 127.4, 72.8, 70.3, 65.2, 53.7, 35.5, 31.7, 31.3, 29.7, 29.6, 29.1 (×2), 28.5, 28.4, 25.9, 25.8, 25.5, 24.2, 22.6, 22.5, 19.8, 14.0, 13.9. MS (ESI): m/z [M+Na]⁺ 527.3712 for C₃₁H₅₂O₅Na.

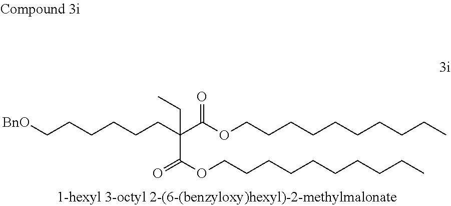 [SMILES: CCCCCCCCCCOC(=O)C(CC)(CCCCCCOCc1ccccc1)C(=O)OCCCCCCCCCC]

Compound 3i was synthesized according to the general step 3 procedure in scheme 1. The title compound was obtained as a colorless oil. ¹H NMR (600 MHz, CDCl₃) δ 7.35-7.27 (m, 5H), 4.48 (s, 2H), 4.09 (t, J=6.6 Hz, 4H), 3.46 (t, J=6.6 Hz, 2H), 1.95-1.87 (m, 4H), 1.65-1.57 (m, 6H), 1.38-1.26 (m, 30H), 0.88 (t, J=6.6 Hz, 6H), 0.80 (t, J=6.6 Hz, 3H). ¹³C NMR (150 MHz, CDCl₃) δ 172.0, 138.6, 128.4, 127.6, 127.5, 73.0, 70.0, 65.2, 58.1, 31.9, 31.5, 30.1, 29.6 (×2), 29.4, 29.3, 28.6, 25.9, 25.2, 22.7, 20.7, 14.1, 8.5. MS (ESI): m/z [M⁺+H]⁺ 575.4675 for C₃₋₆H₆₃O₅.

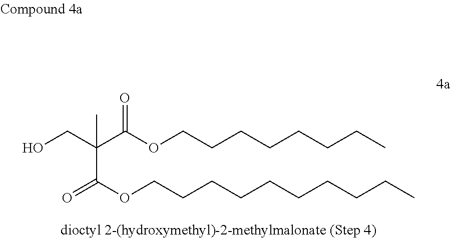 [SMILES: CCCCCCCCCCOC(=O)C(C)(CO)C(=O)OCCCCCCCC]

A mixture solution of dioctyl 2-((benzyloxy)methyl)-2-methylmalonate (1.4 g, 3.02 mmol) and Pd/C (cat.) in AcOH/MeOH (4/1) was stirred at R.T under H₂ atmosphere overnight. After filtration and evaporation, the residue was dissolved in DCM and washed with sat. NaHCO_{3(aq)} and dried over MgSO₄. The desired product 4a (1.07 g, 2.87 mmol, 95%) as a colorless oil was obtained without further purification. ¹H NMR (600 MHz, CDCl₃) δ 4.13 (t, J=6.6 Hz, 4H), 3.83 (s, 2H), 1.64-1.59 (m, 4H), 1.43 (s, 3H), 1.28-1.26 (m, 20H), 0.87 (t, J=7.2 Hz, 6H). ¹³C NMR (150 MHz, CDCl₃) δ 171.7, 66.9, 65.7, 55.9, 31.8, 29.1 (×2), 28.4, 25.8, 22.6, 17.6, 14.1.

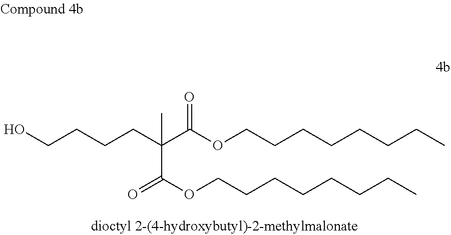 [SMILES: CCCCCCCCOC(=O)C(C)(CCCCO)C(=O)OCCCCCCCC]

Compound 4b was synthesized according to the general step 4 procedure in scheme 1. The title compound was obtained as a colorless oil. ¹¹H NMR (600 MHz, CDCl₃) δ 4.09 (t, J=6.6 Hz, 4H), 3.64 (t, J=6.6 Hz, 2H), 1.88-1.85 (m, 2H), 1.62-1.55 (m, 8H), 1.40 (s, 3H), 1.29-1.27 (m, 20H), 0.87 (t, J=7.2 Hz, 6H). ¹³C NMR (150 MHz, CDCl3) δ 172.5, 65.4, 62.4, 53.8, 35.2, 32.8, 31.8, 29.2 (×2), 28.5, 25.8, 22.6, 20.6, 19.9, 14.1.

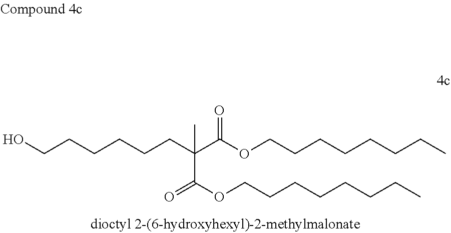 [SMILES: CCCCCCCCOC(=O)C(C)(CCCCCCO)C(=O)OCCCCCCCC]

Compound 4c was synthesized according to the general step 4 procedure in scheme 1. The title compound was obtained as a colorless oil. ¹H NMR (600 MHz, CDCl₃) δ 4.09 (t, J=6.6 Hz, 4H), 3.63 (t, J=6.6 Hz, 2H), 1.86-1.83 (m, 2H), 1.62-1.54 (m, 6H), 1.39 (s, 3H), 1.31-1.27 (m, 26H), 0.88 (t, J=7.2 Hz, 6H). ¹³C NMR (150 MHz, CDCl₃) δ 65.3, 62.9, 53.8, 35.5, 32.7, 31.8, 29.7 (×2), 29.2, 28.5, 25.6, 25.5, 24.3, 22.6, 19.9, 14.1.

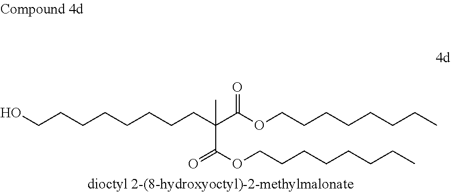 [SMILES: CCCCCCCCOC(=O)C(C)(CCCCCCCCO)C(=O)OCCCCCCCC]

Compound 4d was synthesized according to the general step 4 procedure in scheme 1. The title compound was obtained as a colorless oil. ¹H NMR (600 MHz, CDCl₃) δ 4.11-4.07 (m, 4H), 3.63 (t, J=6.6 Hz, 2H), 1.85-1.82 (m, 2H), 1.61-1.54 (m, 6H), 1.39 (s, 3H), 1.29-1.27 (m, 30H), 0.88 (t, J=7.2 Hz, 6H). ¹³C NMR (150 MHz, CDCl₃) δ 65.3, 63.0, 53.8, 35.6, 32.8, 31.8, 29.8 (×2), 29.34, 29.3, 29.2, 28.5, 25.9, 25.7, 24.3, 22.6, 19.9, 14.1.

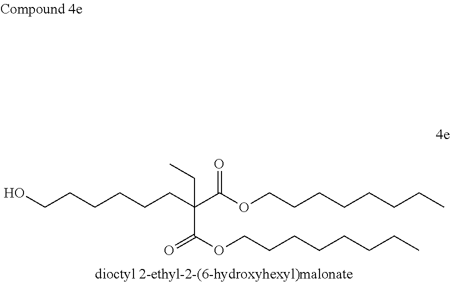 [SMILES: CCCCCCCCOC(=O)C(CC)(CCCCCCO)C(=O)OCCCCCCCC]

Compound 4e was synthesized according to the general step 4 procedure in scheme 1. The title compound was obtained as a colorless oil. ¹H NMR (600 MHz, CDCl₃) δ 4.08-4.06 (m, 4H), 3.61-3.59 (m, 2H), 1.89 (q, J=7.2 Hz, 2H), 1.85-1.83 (m, 2H), 1.59-1.52 (m, 6H), 1.32-1.22 (m, 24H), 1.15-1.13 (m, 2H), 0.87-0.85 (m, 6H), 0.79 (t, J=7.2 Hz, 3H). ¹³C NMR (150 MHz, CDCl₃) δ 172.1, 65.2, 63.0, 58.1, 32.7, 31.8, 31.7, 29.7, 29.3, 29.2, 28.6, 25.9, 25.7, 25.3, 24.0, 22.7, 14.1, 8.5.

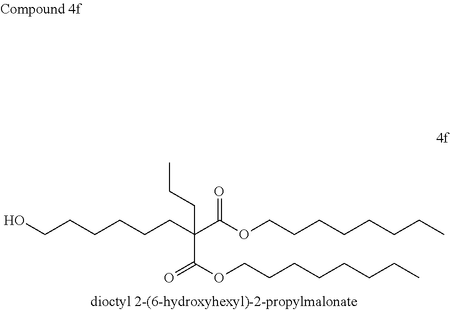 [SMILES: CCCCCCCCOC(=O)C(CCC)(CCCCCCO)C(=O)OCCCCCCCC]

Compound 4f was synthesized according to the general step 4 procedure in scheme 1. The title compound was obtained as a colorless oil. ¹H NMR (600 MHz, CDCl₃) δ 4.08 (t, J=6.6 Hz, 4H), 3.62 (t, J=6.6 Hz, 2H), 1.87-1.82 (m, 4H), 1.64-1.53 (m, 6H), 1.36-1.25 (m, 26H), 1.20-1.13 (m, 2H), 0.91 (t, J=7.2 Hz, 3H), 0.88 (t, J=7.2 Hz, 6H). ¹³C NMR (150 MHz, CDCl₃) δ 172.0, 65.1, 62.9, 57.6, 34.5, 32.6, 31.7, 29.6, 29.2, 29.1, 28.5, 25.8, 25.7, 25.4, 24.0, 22.6, 17.4, 14.4, 14.0.

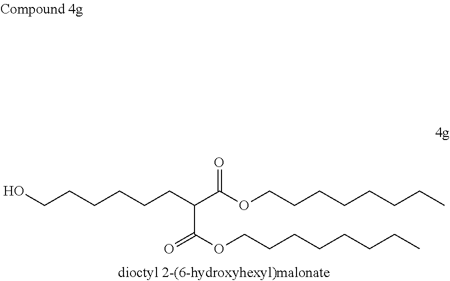 [SMILES: CCCCCCCCOC(=O)C(CCCCCCO)C(=O)OCCCCCCCC]

Compound 4g was synthesized according to the general step 4 procedure in scheme 1. The tide compound was obtained as a colorless oil. ¹H NMR (600 MHz, CDCl₃) δ 4.15-4.08 (m, 4H), 3.63 (t, J=6.6 Hz, 2H), 3.31 (t, J=7.8 Hz, 1H), 1.91-1.87 (m, 2H), 1.64-1.53 (m, 8H), 1.35-1.33 (m, 24H), 0.87 (t, J=7.2 Hz, 6H). ¹³C NMR (150 MHz, CDCl₃) δ 169.6, 65.4, 62.9, 52.1, 32.6, 31.8, 29.2 (×2), 29.0, 28.6, 28.5, 27.3, 25.8, 25.4, 22.6, 14.1.

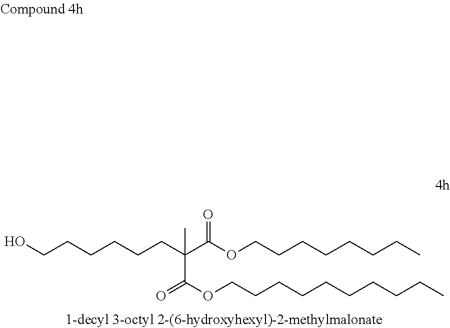 [SMILES: CCCCCCCCCCOC(=O)C(C)(CCCCCCO)C(=O)OCCCCCCCC]

Compound 4h was synthesized according to the general step 4 procedure in scheme 1. The title compound was obtained as a colorless oil. ¹H NMR (600 MHz, CDCl₃) δ 4.09-4.07 (m, 4H), 3.61 (t, J=6.6 Hz, 2H), 1.85-1.82 (m, 2H), 1.60-1.50 (m, 6H), 1.38 (s, 3H), 1.37-1.21 (m, 30H), 0.88-0.86 (m, 6H). ¹³C NMR (150 MHz, CDCl₃) δ 172.6, 65.4, 63.0, 53.8, 35.6, 32.7, 32.0, 31.8, 29.7, 29.6, 29.4, 29.3, 29.26, 29.24, 28.6, 25.9, 25.6, 24.3, 22.73, 22.70, 20.0, 14.2, 14.1.

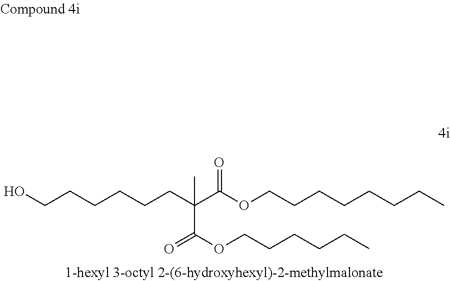 [SMILES: CCCCCCCCOC(=O)C(C)(CCCCCCO)C(=O)OCCCCCC]

Compound 4i was synthesized according to the general step 4 procedure in scheme 1. The title compound was obtained as a colorless oil. ¹H NMR (600 MHz, CDCl₃) δ 4.09 (t, J=6.6 Hz, 4H), 3.62 (t, J=6.6 Hz, 2H), 1.85-1.83 (m, 2H), 1.62-1.59 (m, 6H), 1.58-1.52 (m, 2H), 1.39 (s, 3H), 1.30-1.26 (m, 20H), 0.89-0.86 (m, 6H). ¹³C NMR (150 MHz, CDCl₃) δ 172.6, 65.3 (×2), 62.9, 53.8, 35.5, 32.7, 31.8, 31.4, 29.7, 29.2 (×2), 28.5, 28.4, 25.9, 25.5 (×2), 24.3, 22.6, 22.5, 19.9, 14.1, 14.0.

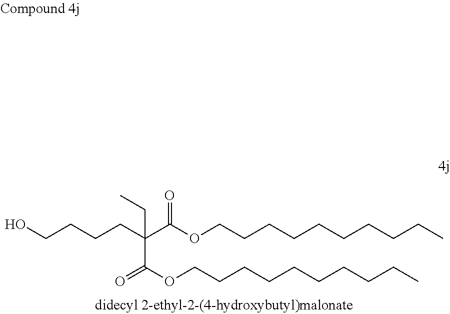 [SMILES: CCCCCCCCCCOC(=O)C(CC)(CCCCO)C(=O)OCCCCCCCCCC]

Compound 4j was synthesized according to the general step 4 procedure in scheme 1. The title compound was obtained as a colorless oil. ¹H NMR (600 MHz, CDCl₃) δ 4.09 (t, J=6.6 Hz, 4H), 3.63 (t, J=6.6 Hz, 2H), 1.95-1.87 (m, 4H), 1.61-1.55 (m, 6H), 1.34-1.24 (m, 30H), 0.87 (t, J=6.6 Hz, 6H), 0.81 (t, J=6.6 Hz, 3H). ¹³C NMR (150 MHz, CDCl₃) δ 171.9, 65.2, 62.5, 58.1, 32.8, 31.9, 31.4, 29.6 (×2), 29.3, 29.2, 28.5, 25.9, 25.4, 22.7, 20.3, 14.1, 8.5.

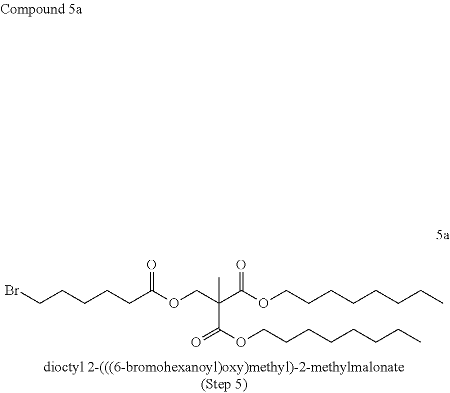 [SMILES: CCCCCCCCOC(=O)C(C)(COC(=O)CCCCCBr)C(=O)OCCCCCCCC]

A mixture solution of compound 4a (440 mg, 1.18 mmol) and 6-bromohexanoic acid (230 mg, 1.18 mmol) in dicholomethane (DCM) was stirred at 0° C., and then the addition of 1-ethyl-3-(3-dimethylaminopropyl)carbodiimide (EDCI) (275 mg, 1.42 mmol) and 4-dimethylaminopyridine (DMAP) (30 mg, 0.24 mmol). After stirring at R.T overnight, the reaction solution was washed with 2N HCl(aq) and brine and dried over MgSO₄. After evaporation, the residue was purified by silica gel column chromatography with ethyl acetate/hexane (EA/Hex) (1/25) to yield the desired product 5a (520 mg, 0.95 mmol, 80%) as a colorless oil. ¹H NMR (600 MHz, CDCl₃) δ 4.45 (s, 2H), 4.12 (t, J=6.6 Hz, 4H), 3.39 (t, J=6.6 Hz, 2H), 2.31 (t, J=7.2 Hz, 2H), 1.88-1.83 (m, 2H), 1.65-1.59 (m, 6H), 1.48 (s, 3H), 1.47-1.44 (m, 2H), 1.43-1.26 (m, 22H), 0.88 (t, J=7.2 Hz, 6H). ¹³C NMR (150 MHz, CDCl₃) δ 172.8, 170.0, 66.4, 65.9, 53.9, 33.9, 33.4, 32.4, 31.9, 29.3(×2), 28.5, 27.7, 25.9, 24.1, 22.7, 18.4, 14.2.

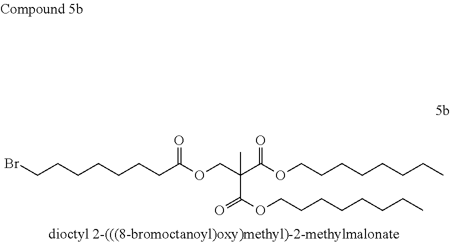 [SMILES: CCCCCCCCOC(=O)C(C)(COC(=O)CCCCCCCBr)C(=O)OCCCCCCCC]

Compound 5b was synthesized according to the general step 5 procedure in scheme 1. The title compound was obtained as a colorless oil. ¹H NMR (600 MHz, CDCl₃) δ 4.44 (s, 2H), 4.12 (t, J=6.6 Hz, 4H), 3.40 (t, J=6.6 Hz, 2H), 2.29 (t, J=7.2 Hz, 2H), 1.87-1.82 (m, 2H), 1.63-1.59 (m, 6H), 1.48 (s, 3H), 1.44-1.40 (m, 2H), 1.33-1.27 (m, 26H), 0.88 (t, J=7.2 Hz, 6H). ¹³C NMR (150 MHz, CDCl₃) δ 173.1, 170.0, 66.3, 65.9, 53.9, 34.1, 33.9, 32.8, 31.9, 29.3(×2), 29.0, 28.5, 28.4, 28.0, 25.9, 24.8, 22.7, 18.4, 14.2. MS (ESI): m/z [M+Na]⁺ 599.2912, [M+Na]²⁺ 601.2890 for C₂₉H₅₃O₆BrNa.

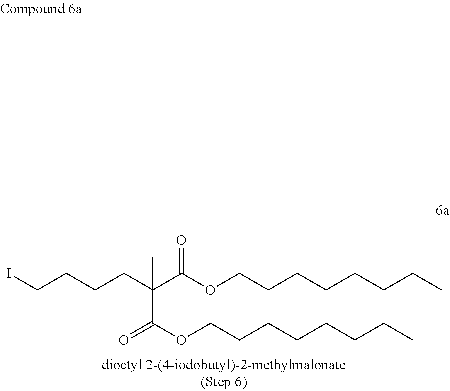 [SMILES: CCCCCCCCOC(=O)C(C)(CCCCI)C(=O)OCCCCCCCC]

A mixture solution of compound 4b (975 mg, 2.202 mmol), PPh₃ (635 mg, 2.242 mmol) and imidazole (165 mg, 2.242 mmol) in DCM was stirred at 0° C., followed by the addition of I₂ (670 mg, 2.643 mmol). The reaction solution was allowed to warm to rt and stirred overnight. After washing with sat. Na₂S₂O_{3(aq)} and brine, the residue was dried over MgSO₄ and evaporated under reduced pressure. The crude product was purified by silica gel column chromatography with EA/Hex (1/25) to yield the desired product 6a (1.096 g, 90%) as a colorless oil. ¹H NMR (600 MHz, CDCl₃) δ 4.10 (t, J=6.6 Hz, 4H), 3.18 (t, J=6.6 Hz, 2H), 1.87-1.80 (m, 4H), 1.62-1.58 (m, 4H), 1.41 (s, 3H), 1.34-1.22 (m, 22H), 0.88 (t, J=7.2 Hz, 6H). ¹³C NMR (150 MHz, CDCl₃) δ 172.3, 65.4, 53.6, 34.4, 33.5, 31.8, 29.2, 28.5, 25.8, 25.3, 22.6, 19.9, 14.1, 6.2.

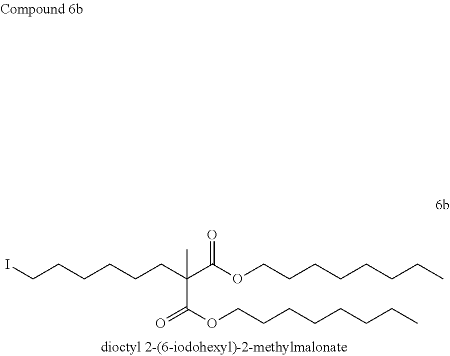 [SMILES: CCCCCCCCOC(=O)C(C)(CCCCCCI)C(=O)OCCCCCCCC]

Compound 6b was synthesized according to the general step 6 procedure in scheme 1. The title compound was obtained as a colorless oil. ¹H NMR (600 MHz, CDCl₃) δ 4.10-4.07 (m, 4H), 3.16 (t, J=6.6 Hz, 2H), 1.84-1.77 (m, 4H), 1.62-1.57 (m, 4H), 1.40-1.36 (m, 2H), 1.38 (s, 3H), 1.31-1.20 (m, 24H), 0.87 (t, J=7.2 Hz, 6H). ¹³C NMR (150 MHz, CDCl₃) δ 172.6, 65.4, 53.8, 35.5, 33.5, 31.8, 30.3, 29.3, 29.2, 28.9, 28.6, 25.9, 24.2, 22.7, 20.0, 14.1, 6.9. MS (ESI): m/z [M+Na]⁺ 561.2406 for C₂₅H₄₇O₄NaI.

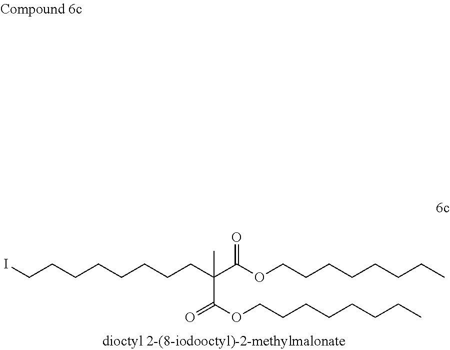 [SMILES: CCCCCCCCOC(=O)C(C)(CCCCCCCCI)C(=O)OCCCCCCCC]

Compound 6c was synthesized according to the general step 6 procedure in scheme 1. The title compound was obtained as a colorless oil. ¹H NMR (600 MHz, CDCl3) δ 4.11-4.04 (m, 4H), 3.16 (t, J=7.2 Hz, 2H), 1.83-1.76 (m, 4H), 1.61-1.56 (m, 4H), 1.37 (s, 3H), 1.37-1.34 (m, 2H), 1.27-1.19 (m, 28H), 0.86 (t, J=7.2 Hz, 6H). ¹³C NMR (150 MHz, CDCl₃) δ 172.6 65.3, 53.8, 35.6, 33.5, 31.8, 30.5, 29.8, 29.23, 29.21 (×2), 28.55, 28.5, 25.9, 24.3, 22.7, 19.9, 14.1, 7.1. MS (ESI): m/z [M+Na]⁺ 603.2876 for C₂₈H₅₃O₄NaI.

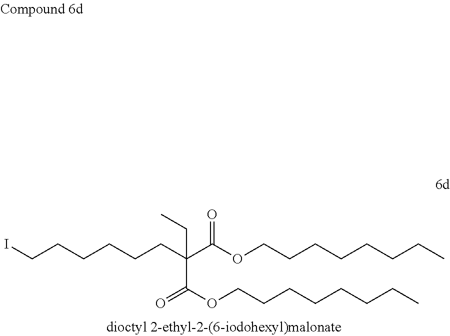 [SMILES: CCCCCCCCOC(=O)C(CC)(CCCCCCI)C(=O)OCCCCCCCC]

Compound 6d was synthesized according to the general step 6 procedure in scheme 1. The title compound was obtained as a colorless oil. ¹H NMR (600 MHz, CDCl₃) δ 4.09 (t, J=6.6 Hz, 4H), 3.16 (t, J=7.2 Hz, 2H), 1.91 (q, J=7.2 Hz, 2H), 1.86-1.83 (m, 2H), 1.81-1.78 (m, 2H), 1.62-1.58 (m, 4H), 1.40-1.37 (m, 2H), 1.33-1.24 (m, 22H), 1.16-1.14 (m, 2H), 0.87 (t, J=7.2 Hz, 6H), 0.80 (t, J=7.8 Hz, 3H). ¹³C NMR (150 MHz, CDCl₃) δ 172.0, 65.2, 58.1, 33.5, 31.9, 31.7, 30.4, 29.3, 29.2, 28.9, 28.6, 26.0, 25.3, 23.9, 22.7, 14.2, 8.5, 6.9.

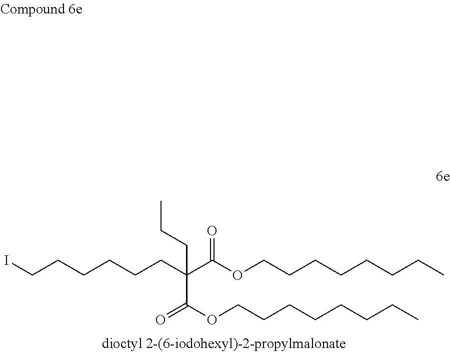 [SMILES: CCCCCCCCOC(=O)C(CCC)(CCCCCCI)C(=O)OCCCCCCCC]

Compound 6e was synthesized according to the general step 6 procedure in scheme 1. The title compound was obtained as a colorless oil. ¹H NMR (600 MHz, CDCl₃) δ 4.09 (t, J=6.6 Hz, 4H), 3.16 (t, J=6.6 Hz, 2H), 1.88-1.84 (m, 4H), 1.62-1.57 (m, 6H), 1.38-1.26 (m, 26H), 1.18-1.12 (m, 2H). ¹³C NMR (150 MHz, CDCl₃) δ 172.0, 65.1, 57.6, 34.6, 33.4, 32.2, 31.8, 30.3, 29.2, 29.1, 28.8, 28.5, 25.9, 23.9, 22.6, 17.4, 14.4, 14.1, 6.9.

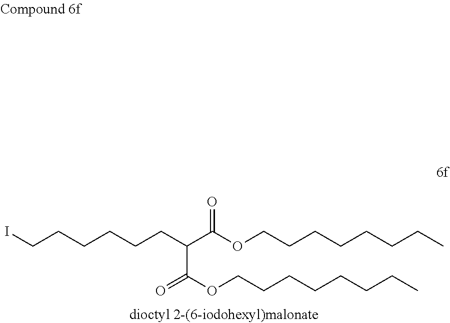 [SMILES: CCCCCCCCOC(=O)C(CCCCCCI)C(=O)OCCCCCCCC]

Compound 6f was synthesized according to the general step 6 procedure in scheme 1. The title compound was obtained as a colorless oil. ¹H NMR (600 MHz, CDCl₃) δ 4.13-4.06 (m, 4H), 3.30-3.26 (m, 1H), 3.16-3.12 (m, 2H), 1.87-1.85 (m, 2H), 1.81-1.76 (m, 2H), 1.61-1.58 (m, 4H), 1.36-1.25 (m, 26H), 0.87-1.83 (m, 6H). ¹³C NMR (150 MHz, CDCl₃) δ 169.6, 65.5, 52.1, 33.4, 31.8, 30.2, 29.2 (×2), 28.7, 28.6, 28.2, 27.2, 25.9, 22.7, 14.1, 6.9.

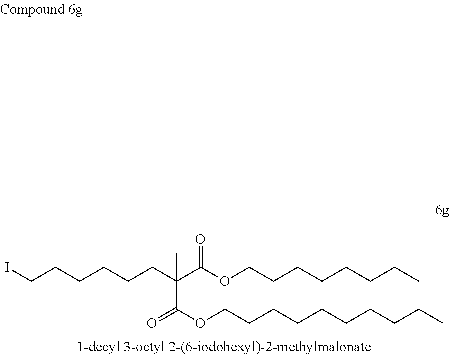 [SMILES: CCCCCCCCCCOC(=O)C(C)(CCCCCCI)C(=O)OCCCCCCCC]

Compound 6g was synthesized according to the general step 6 procedure in scheme 1. The title compound was obtained as a colorless oil. ¹H NMR (600 MHz, CDCl₃) δ 4.09-4.03 (m, 4H), 3.13 (t, J=6.6 Hz, 2H), 1.82-1.76 (m, 4H), 1.60-1.55 (m, 4H), 1.40-1.32 (m, 5H), 1.31-1.18 (m, 28H), 0.86-0.84 (m, 6H). ¹³C NMR (150 MHz, CDCl₃) δ 172.5, 65.3, 53.7, 35.5, 33.4, 31.9, 31.8, 30.3, 29.6, 29.3, 29.22, 29.21, 29.18, 28.8, 28.5, 25.9, 24.1, 22.7, 22.6, 19.9, 14.11, 14.09, 6.8.

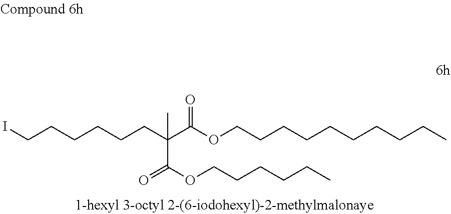 [SMILES: CCCCCCCCCCOC(=O)C(C)(CCCCCCI)C(=O)OCCCCCC]

Compound 6h was synthesized according to the general step 6 procedure in scheme 1. The title compound was obtained as a colorless oil. ¹H NMR (600 MHz, CDCl₃) δ 4.11-4.05 (m, 4H), 3.15 (t, J=6.6 Hz, 2H), 1.84-1.77 (m, 4H), 1.60-1.56 (m, 4H), 1.40-1.35 (m, 5H), 1.33-1.18 (m, 20H), 0.88-0.85 (m, 6H). ¹³C NMR (150 MHz, CDCl₃) δ 172.6, 65.4, 53.8, 35.5, 33.5, 31.8, 31.4, 30.3, 29.3, 29.2, 28.9, 28.6, 28.5, 25.9, 25.6, 24.2, 22.7, 22.6, 20.0, 14.1, 14.0, 7.0.

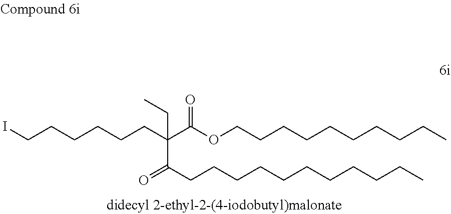 [SMILES: CCCCCCCCCCCC(=O)C(CC)(CCCCCCI)C(=O)OCCCCCCCCCC]

Compound 6i was synthesized according to the general step 6 procedure in scheme 1. The title compound was obtained as a colorless oil. ¹H NMR (600 MHz, CDCl₃) δ 4.10 (t, J=6.6 Hz, 4H), 3.17 (t, J=6.6 Hz, 2H), 1.95-1.91 (q, J=6.6 Hz, 2H), 1.87-1.81 (m, 4H), 1.63-1.58 (m, 4H), 1.32-1.25 (m, 30H), 0.87 (t, J=6.6 Hz, 6H), 0.82 (t, J=6.6 Hz, 3H), 13C NMR (150 MHz, CDCl3) δ 171.8, 65.4, 58.0, 33.7, 32.0, 30.6, 29.7 (×2), 29.4, 29.3, 28.6, 26.0, 25.4, 25.0, 22.8, 14.2, 8.6, 6.3.

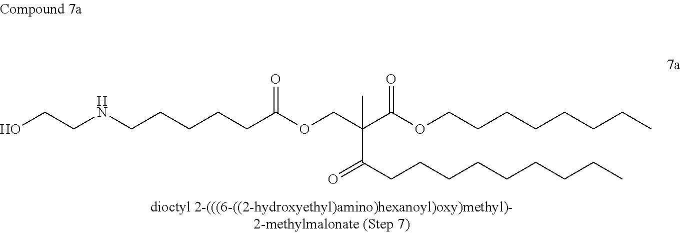 [SMILES: CCCCCCCCCC(=O)C(C)(COC(=O)CCCCCNCCO)C(=O)OCCCCCCCC]

A mixture solution of compound 5a (490 mg, 0.89 mmol) and KI (150 mg, 0.89 mmol) in MeCN/DCM was stirred at R.T. and then the addition of ethanolamine (1.6 mL, 26.75 mmol). After stirring for 4 h, the solvent was removed and the residue was dissolved in DCM. The organic solvent was washed with water and brine and dried over MgSO₄. The crude product was purified by silica gel column chromatography with 10% MeOH/1% NH₄OH in DCM to yield the desired product 7a (325 mg, 0.61 mmol, 69%) as a colorless oil. ¹H NMR (600 MHz, CDCl₃) δ 4.44 (s, 2H), 4.12 (t, J=6.6 Hz, 4H), 3.64 (t, J=5.4 Hz, 2H), 2.77 (t, J=5.4 Hz, 2H), 2.62 (t, J=6.6 Hz, 2H), 2.30 (t, J=7.2 Hz, 2H), 2.18 (br. s, 2H), 1.62-1.59 (m, 6H), 1.52-1.49 (m, 2H), 1.48 (s, 3H), 1.36-1.26 (m, 22H), 0.88 (t, J=7.2 Hz, 6H). ¹³C NMR (150 MHz, CDCl₃) δ 172.9, 169.9, 66.2, 65.8, 60.7, 53.8, 51.2, 49.2, 33.9, 31.7, 29.6, 29.1(×2), 28.4, 26.7, 25.7, 24.6, 22.6, 18.2, 14.0. MS (ESI): m/z [M+H]⁺ 530.4044 for C₂₉H₅₆NO₇.

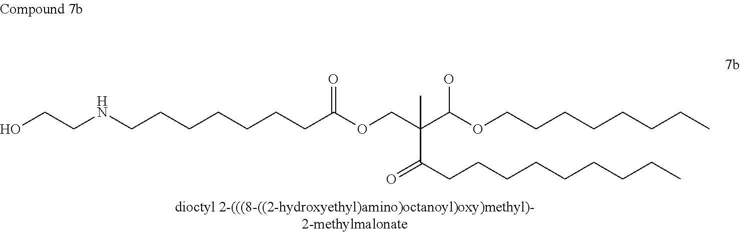 [SMILES: CCCCCCCCCC(=O)C(C)(COC(=O)CCCCCCCNCCO)C(O)OCCCCCCCC]

Compound 7b was synthesized according to the general step 7 procedure in scheme 1. The title compound was obtained as a colorless oil. ¹H NMR (600 MHz, CDCl3) δ 4.44 (s, 2H), 4.12 (t, J=6.6 Hz, 4H), 3.63 (t, J=5.4 Hz, 2H), 2.77 (t, J=5.4 Hz, 2H), 2.60 (t, J=7.2 Hz, 2H), 2.28 (t, J=7.2 Hz, 2H), 1.63-1.58 (m, 6H), 1.48-1.46 (m, 2H), 1.46 (s, 3H), 1.30-1.26 (m, 26H), 0.88 (t, J=7.2 Hz, 6H). ¹³C NMR (150 MHz, CDCl₃) δ 173.2, 170.0, 66.2, 65.9, 60.9, 53.9, 51.0, 49.4, 34.1, 31.8, 30.1, 29.2 (×2), 29.1, 29.0, 28.5, 27.1 25.8, 24.8, 22.7, 18.3, 14.1. MS (ESI): m/z [M+H]⁺ 558.4363 for C₃₁H₆₀NO₇.

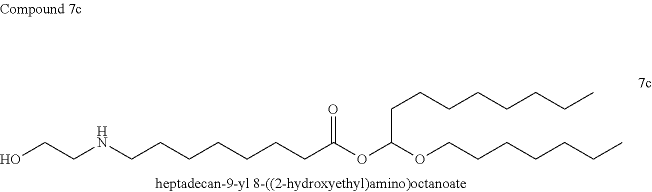 [SMILES: CCCCCCCCC(OCCCCCCC)OC(=O)CCCCCCCNCCO]

A mixture solution of 8-bromooctanoic acid (0.9 g, 4.03 mmol) and heptadecan-9-ol (1.04 g, 4.03 mmol) in dicholomethane (DCM) was stirred at 0° C., and then the addition of 1-ethyl-3-(3-dimethylaminopropyl)carbodiimide (EDCI) (0.93 g, 4.84 mmol) and 4-dimethylaminopyridine (DMAP) (0.1 mg, 0.8 mmol). After stirring at R.T for 4 hours, the reaction solution was washed with 2N HCl_{(aq)} and brine and dried over MgSO₄. After evaporation, the residue was purified by silica gel column chromatography with ethyl acetate/hexane (EA/Hex) (1/20) to yield the precursor of compound 7c (1.58 g, 3.42 mmol, 85%).

Compound 7c was synthesized according to the general step 7 procedure in scheme 1. The title compound was obtained as a colorless oil. ¹H NMR (600 MHz, CDCl₃) δ 4.84-4.79 (m, 1H), 3.60 (t, J=5.4 Hz, 2H), 2.71 (t, J=5.4 Hz, 2H), 2.56 (t, J=7.2 Hz, 2H), 2.22 (t, J=7.2 Hz, 2H), 1.58-1.56 (m, 2H), 1.46-1.45 (m, 6H), 1.27-1.21 (m, 30H), 0.83 (t, J=7.2 Hz, 6H). ¹³C NMR (150 MHz, CDCl₃) δ 173.6, 74.1, 60.6, 51.3, 49.6, 34.6, 34.1, 31.8, 29.9, 29.5, 29.48, 29.2, 29.17, 29.1, 27.1, 25.3, 25.1, 22.6, 14.1. MS (ESI): m/z [M+H]⁺ 442.4264 for C₂₇H₅₆NO₃.

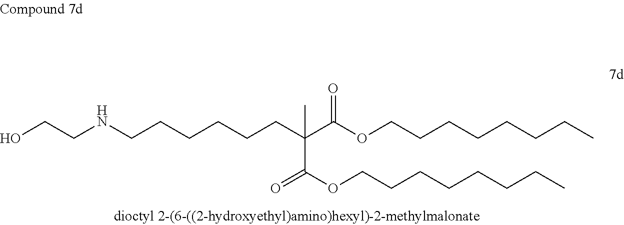 [SMILES: CCCCCCCCOC(=O)C(C)(CCCCCCNCCO)C(=O)OCCCCCCCC]

Compound 7d was synthesized according to the general step 7 procedure in scheme 1. The title compound was obtained as a colorless oil. ¹H NMR (600 MHz, CDCl₃) δ 4.08 (t, J=6.6 Hz, 4H), 3.63 (t, J=4.8 Hz, 2H), 2.76 (t, J=4.8 Hz, 2H), 2.60 (t, J=7.2 Hz, 2H), 1.86-1.82 (m, 2H), 1.62-1.57 (m, 4H), 1.48-1.44 (m, 2H), 1.38 (s, 3H), 1.31-1.25 (m, 26H), 0.86-0.84 (m, 6H). ¹³C NMR (150 MHz, CDCl₃) δ 172.6, 65.3, 60.8, 53.7, 51.1, 49.5, 35.5, 31.8, 30.0, 29.8, 29.2 (×2), 28.5, 27.1, 25.6, 24.3, 22.6, 19.9, 14.1.

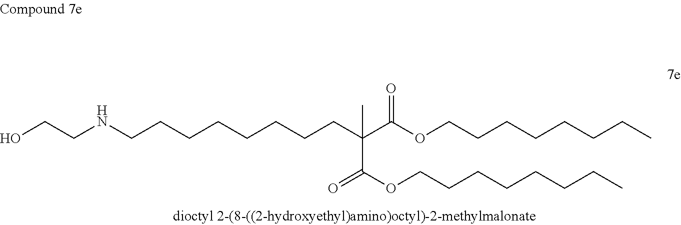 [SMILES: CCCCCCCCOC(=O)C(C)(CCCCCCCCNCCO)C(=O)OCCCCCCCC]

Compound 7e was synthesized according to the general step 7 procedure in scheme 1. The title compound was obtained as a colorless oil. ¹H NMR (600 MHz, CDCl₃) δ 4.10-4.04 (m, 4H), 3.64 (t, J=4.8 Hz, 2H), 2.76 (t, J=4.8 Hz, 2H), 2.61 (t, J=7.2 Hz, 2H), 1.83-1.80 (m, 2H), 1.61-1.56 (m, 4H), 1.49-1.45 (m, 2H), 1.37 (s, 3H), 1.34-1.18 (m, 30H), 0.86 (t, J=7.2 Hz, 2H). ¹³C NMR (150 MHz, CDCl₃) δ 172.6, 65.2, 60.6, 53.7, 51.1, 49.5, 35.54, 31.8, 29.8 (×2), 29.4, 29.3, 29.2 (×2), 28.5, 27.2, 25.8, 24.3, 22.6, 19.9, 14.0. MS (ESI): m/z [M+H]⁺ 514.4461 for C₃₀H₆₀NO₅.

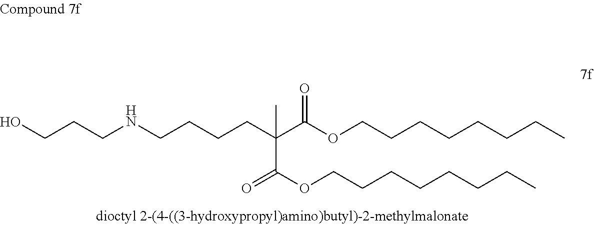 [SMILES: CCCCCCCCOC(=O)C(C)(CCCCNCCCO)C(=O)OCCCCCCCC]

Compound 7f was synthesized according to the general step 7 procedure in scheme 1. The title compound was obtained as a colorless oil. ¹H NMR (600 MHz, CDCl3) δ 4.07 (t, J=6.6 Hz, 4H), 3.77 (t, J=5.4 Hz, 2H), 2.85 (t, J=5.4 Hz, 2H), 2.59 (t, J=7.2 Hz, 2H), 1.84-1.81 (m, 2H), 1.68-1.66 (m, 2H), 1.59-1.56 (m, 4H), 1.48-1.45 (m, 2H), 1.37 (s, 3H), 1.28-1.24 (m, 22H), 0.86 (t, J=6.6 Hz, 6H). ¹³C NMR (150 MHz, CDCl₃) δ 172.5, 65.4, 64.3, 53.8, 50.0, 49.5, 35.4, 31.8, 30.6, 30.0, 29.2 (×2), 28.5, 25.9, 22.7, 22.1, 19.9, 14.1. MS (ESI): m/z [M+H]⁺ 472.3994 for C₂₇H₅₄NO₅.

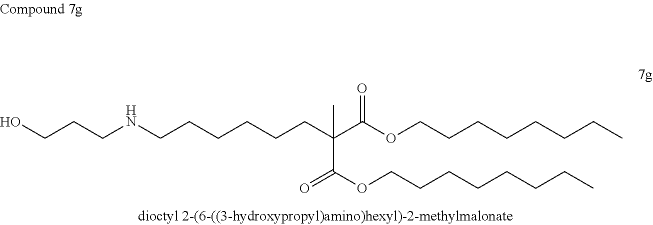 [SMILES: CCCCCCCCOC(=O)C(C)(CCCCCCNCCCO)C(=O)OCCCCCCCC]

Compound 7g was synthesized according to the general step 7 procedure in scheme 1. The title compound was obtained as a colorless oil. ¹H NMR (600 MHz, CDCl₃) δ 4.05 (t, J=6.6 Hz, 4H), 3.78-3.76 (m, 2H), 2.88-2.86 (m, 2H), 2.61-2.59 (m, 2H), 1.79-1.78 (m, 2H), 1.71-1.69 (m, 2H), 1.57-1.54 (m, 4H), 1.48-1.44 (m, 2H), 1.34 (s, 3H), 1.27-1.17 (m, 26H), 0.85-0.83 (m, 6H). ¹³C NMR (150 MHz, CDCl₃) δ 172.6, 65.3, 63.8, 53.8, 49.6, 49.5, 35.5, 31.8, 30.2, 29.7, 29.3, 29.2, 29.1, 28.5, 27.0, 25.9, 24.3, 22.6, 19.9, 14.1. MS (ESI): m/z [M+H]⁺ 500.4310 for C₂₉H₅₈NO₅.

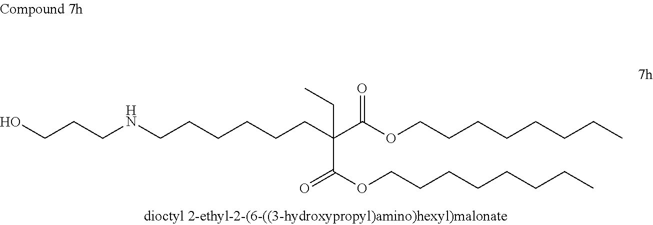 [SMILES: CCCCCCCCOC(=O)C(CC)(CCCCCCNCCCO)C(=O)OCCCCCCCC]

Compound 7h was synthesized according to the general step 7 procedure in scheme 1. The title compound was obtained as a colorless oil. ¹H NMR (600 MHz, CDCl₃) δ 4.08 (t, J=6.6 Hz, 4H), 3.80 (t, J=5.4 Hz, 2H), 2.86 (t, J=5.4 Hz, 2H), 2.58 (t, J=7.2 Hz, 2H), 1.92 (q, J=7.2 Hz, 2H), 1.89-1.83 (m, 2H), 1.69-1.67 (m, 2H), 1.60-1.58 (m, 4H), 1.48-1.42 (m, 2H), 1.35-1.22 (m, 24H), 1.15-1.12 (m, 2H), 0.87 (t, J=6.6 Hz, 6H), 0.80 (t, J=7.2 Hz, 3H). ¹³C NMR (150 MHz, CDCl₃) δ 172.1, 65.2, 64.6, 58.1, 50.2, 49.8, 31.9, 31.7, 30.6, 29.9 (×2), 29.3 (×2), 28.6, 27.1, 26.0, 25.3, 24.0, 22.7, 14.2, 8.5. MS (ESI): m/z [M+H]⁺ 514.4466 for C₃₀H₆₀NO₅.

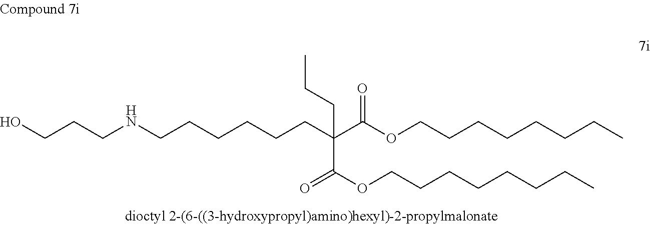 [SMILES: CCCCCCCCOC(=O)C(CCC)(CCCCCCNCCCO)C(=O)OCCCCCCCC]

Compound 7i was synthesized according to the general step 7 procedure in scheme 1. The title compound was obtained as a colorless oil. ¹H NMR (600 MHz, CDCl₃) δ 4.07 (t, J=6.6 Hz, 4H), 3.78 (t, J=4.8 Hz, 2H), 2.88 (t, J=4.8 Hz, 2H), 2.60 (t, J=7.2 Hz, 2H), 1.84-1.81 (m, 4H), 1.72-1.70 (m, 2H), 1.60-1.56 (m, 4H), 1.50-1.44 (m, 2H), 1.31-1.22 (m, 24H), 1.18-1.10 (m, 4H), 0.9 (t, J=7.2 Hz, 3H), 0.86 (t, J=6.6 Hz, 6H). ¹³C NMR (150 MHz, CDCl₃) δ 172.1, 65.2, 64.0, 57.7, 49.8, 49.6, 34.6, 32.3, 31.8, 30.4, 29.8, 29.5, 29.3, 29.2, 28.6, 27.1, 25.9, 24.1, 22.7, 17.5, 14.5, 14.1. MS (ESI): m/z [M+H]⁺ 528.4623 for C₃₁H₆₂NO₅.

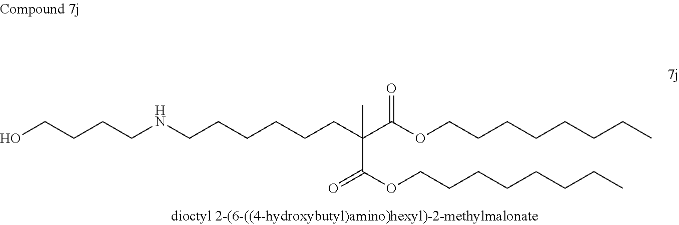 [SMILES: CCCCCCCCOC(=O)C(C)(CCCCCCNCCCCO)C(=O)OCCCCCCCC]

Compound 7j was synthesized according to the general step 7 procedure in scheme 1. The title compound was obtained as a colorless oil. ¹H NMR (600 MHz, CDCl3) δ 4.06 (t, J=6.6 Hz, 4H), 3.55 (t, J=5.4 Hz, 2H), 2.64 (t, J=5.4 Hz, 2H), 2.58 (t, J=7.2 Hz, 2H), 1.82-1.79 (m, 2H), 1.66-1.56 (m, 8H), 1.50-1.45 (m, 2H), 1.36 (s, 3H), 1.29-1.20 (m, 26H), 0.85 (t, J=7.2 Hz, 6H). ¹³C NMR (150 MHz, CDCl₃) δ 172.6, 65.4, 62.6, 53.8, 49.6, 49.4, 35.6, 32.6, 31.8, 29.8, 29.5, 29.3, 29.2, 28.7, 28.6, 27.1, 25.9, 24.3, 22.7, 19.9, 14.1. MS (ESI): m/z [M+H]⁺ 514.4468 for C₃₀H₆₀NO₅.

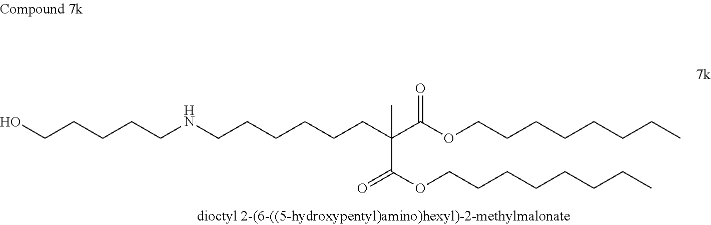 [SMILES: CCCCCCCCOC(=O)C(C)(CCCCCCNCCCCCO)C(=O)OCCCCCCCC]

Compound 7k was synthesized according to the general step 7 procedure in scheme 1. The title compound was obtained as a colorless oil. ¹H NMR (600 MHz, CDCl₃) δ 4.07 (t, J=6.6 Hz, 4H), 3.60 (t, J=6.6 Hz, 2H), 2.58 (t, J=6.6 Hz, 2H), 2.55 (t, J=7.2 Hz, 2H), 1.83-1.80 (m, 2H), 1.60-1.54 (m, 6H), 1.51-1.48 (m, 2H), 1.46-1.43 (m, 2H), 1.40-1.37 (m, 2H), 1.36 (s, 3H), 1.28-1.19 (m, 26H), 0.86 (t, J=7.2 Hz, 6H). ¹³C NMR (150 MHz, CDCl₃) δ 172.6, 65.3, 62.5, 53.8, 50.0, 49.8, 35.6, 32.5, 31.8, 30.0, 29.9, 29.7, 29.2 (×2), 28.5, 27.2, 25.9, 24.3, 23.5, 22.7, 19.9, 14.1. MS (ESI): m/z [M+H]⁺ 528.4618 for C₃₁H₆₂NO₅.

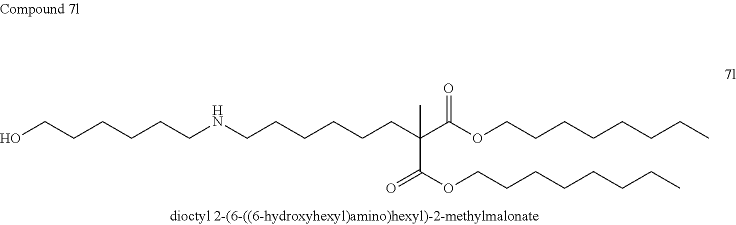 [SMILES: CCCCCCCCOC(=O)C(C)(CCCCCCNCCCCCCO)C(=O)OCCCCCCCC]

Compound 71 was synthesized according to the general step 7 procedure in scheme 1. The title compound was obtained as a colorless oil. ¹H NMR (600 MHz, CDCl₃) δ 4.05 (t, J=6.6 Hz, 4H), 3.58-3.56 (m, 2H), 2.57-2.53 (m, 4H), 1.81-1.78 (m, 2H), 1.58-1.42 (m, 10H), 1.35 (s, 3H), 1.34-1.13 (m, 30H), 0.84 (t, J=7.2 Hz, 6H). ¹³C NMR (150 MHz, CDCl₃) δ 172.6, 65.3, 62.5, 53.8, 49.9, 49.8, 35.5, 32.7, 31.8, 29.8, 29.7 (×2), 29.2 (×2), 28.5, 27.2, 27.1, 25.9, 25.7, 24.3, 22.6, 19.9, 14.1.

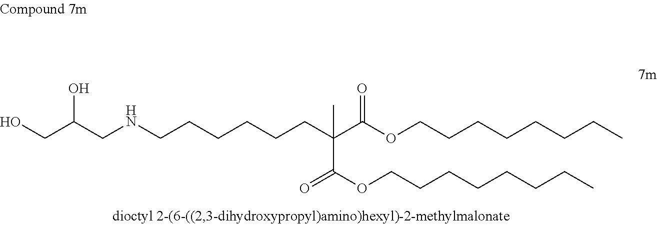 [SMILES: CCCCCCCCOC(=O)C(C)(CCCCCCNCC(O)CO)C(=O)OCCCCCCCC]

Compound 7m was synthesized according to the general step 7 procedure in scheme 1. The title compound was obtained as a colorless oil. ¹H NMR (600 MHz, CDCl₃) δ 4.06 (t, J=6.6 Hz, 4H), 3.75-3.72 (m, 1H), 3.66-3.65 (m, 1H), 3.56-3.53 (m, 1H), 2.75-2.73 (m, 1H), 2.65-2.61 (m, 1H), 2.59-2.52 (m, 2H), 1.82-1.78 (m, 2H), 1.59-1.54 (m, 4H), 1.45-1.42 (m, 2H), 1.34 (s, 3H), 1.28-1.18 (m, 26H), 0.85 (t, J=7.2 Hz, 6H). ¹³C NMR (150 MHz, CDCl₃) δ 1172.6, 69.7, 65.8, 65.3, 53.8, 52.4, 49.9, 35.6, 31.8, 29.9, 29.8, 29.2 (×2), 28.5, 27.0, 25.9, 24.3, 22.7, 19.9, 14.1. MS (ESI): m/z [M+H]⁺ 516.4252 for C₂₉H₅₈NO₆.

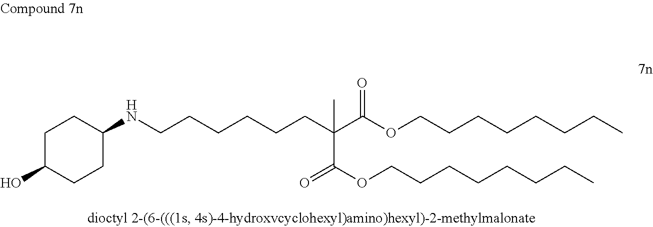 [SMILES: CCCCCCCCOC(=O)C(C)(CCCCCCN[C@H]1CC[C@@H](O)CC1)C(=O)OCCCCCCCC]

Compound 7n was synthesized according to the general step 7 procedure in scheme 1. The title compound was obtained as a colorless oil. ¹H NMR (600 MHz, CDCl₃) δ 4.07 (t, J=6.6 Hz, 4H), 3.95-3.94 (m, 1H), 2.78-2.71 (m, 3H), 1.82-1.70 (m, 8H), 1.63-1.52 (m, 8H), 1.36 (s, 3H), 1.32-1.18 (m, 26H), 0.87-0.84 (m, 6H). ¹³C NMR (150 MHz, CDCl₃) δ172.4, 65.8, 65.2, 55.4, 53.6, 46.3, 35.4, 31.7, 31.0, 29.6, 29.1 (×2), 29.0, 28.4, 27.0, 26.2, 25.7, 24.2, 22.5, 19.8, 14.0. MS (ESI): m/z [M+H]⁺ 540.4614 for C₃₂H₆₂NO₅.

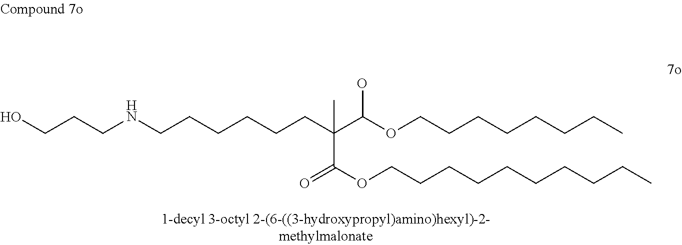 [SMILES: CCCCCCCCCCOC(=O)C(C)(CCCCCCNCCCO)C(O)OCCCCCCCC]

Compound 7o was synthesized according to the general step 7 procedure in scheme 1. The title compound was obtained as a colorless oil. ¹H NMR (600 MHz, CDCl₃) δ 4.07 (t, J=6.6 Hz, 4H), 3.78 (t, J=5.4 Hz, 2H), 2.86 (t, J=5.4 Hz, 2H), 2.59 (t, J=7.2 Hz, 2H), 1.84-1.78 (m, 2H), 1.70-1.66 (m, 2H), 1.61-1.55 (m, 4H), 1.46-1.42 (m, 2H), 1.37 (s, 3H), 1.32-1.18 (m, 30H), 0.87-0.85 (m, 6H). ¹³C NMR (150 MHz, CDCl₃) δ 172.6, 65.3, 64.3, 53.8, 49.9, 49.7, 35.6, 31.9, 31.8, 30.5, 29.8, 29.7, 29.6, 29.3, 29.26, 29.23, 29.22, 28.5, 27.1, 25.9, 24.3, 22.7, 22.6, 19.9, 14.13, 14.11. MS (ESI): m/z [M+H]⁺ 528.4619 for C₃₁H₆₂NO₅.

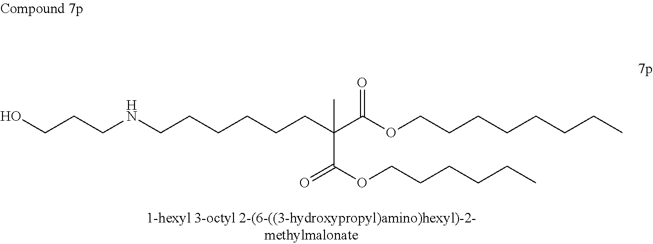 [SMILES: CCCCCCCCOC(=O)C(C)(CCCCCCNCCCO)C(=O)OCCCCCC]

Compound 7p was synthesized according to the general step 7 procedure in scheme 1. The title compound was obtained as a colorless oil. ¹H NMR (600 MHz, CDCl₃) δ 4.07 (t, J=6.6 Hz, 4H), 3.79 (t, J=5.4 Hz, 2H), 2.86 (t, J=5.4 Hz, 2H), 2.58 (t, J=7.2 Hz, 2H), 1.83-1.80 (m, 2H), 1.69-1.66 (m, 2H), 1.60-1.57 (m, 4H), 1.45-1.43 (m, 2H), 1.37 (s, 3H), 1.36-1.19 (m, 22H), 0.87-0.85 (m, 6H). ¹³C NMR (150 MHz, CDCl3) δ 172.6, 65.3, 64.4, 53.8, 50.1, 49.8, 35.6, 31.8, 31.4, 30.5, 29.8, 29.7, 29.24, 29.22, 28.6, 28.5, 27.1, 25.9, 25.6, 24.3, 22.7, 22.6, 19.9, 14.1, 14.0. MS (ESI): m/z [M+H]⁺ 472.3987 for C₂₇H₅₄NO₅

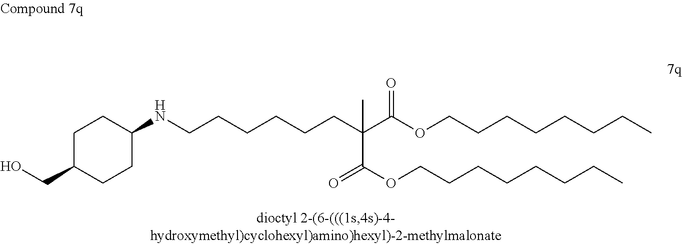 [SMILES: CCCCCCCCOC(=O)C(C)(CCCCCCN[C@H]1CC[C@@H](CO)CC1)C(=O)OCCCCCCCC]

Compound 7q was synthesized according to the general step 7 procedure in scheme 1. The title compound was obtained as a colorless oil. ¹H NMR (600 MHz, CDCl3) δ 4.07 (t, J=6.6 Hz, 4H), 3.51 (d, J=6.6 Hz, 2H), 2.71-2.70 (m, 1H), 2.57 (t, J=7.2 Hz, 2H), 1.82-1.80 (m, 2H), 1.65-1.54 (m, 8H), 1.51-1.48 (m, 6H), 1.36 (s, 3H), 1.31-1.18 (m, 26H), 0.87-0.84 (m, 6H). ¹³C NMR (150 MHz, CDCl₃) δ 172.6, 65.8, 65.3, 54.4, 53.8, 46.8, 37.8, 35.5, 31.8, 29.8, 29.3, 29.21, 29.19, 28.5, 28.3, 27.2, 25.9, 24.3, 24.2, 22.7, 19.9, 14.1. MS (ESI): m/z [M+H]⁺ 554.4787 for C₃₃H₆₄NO₅.

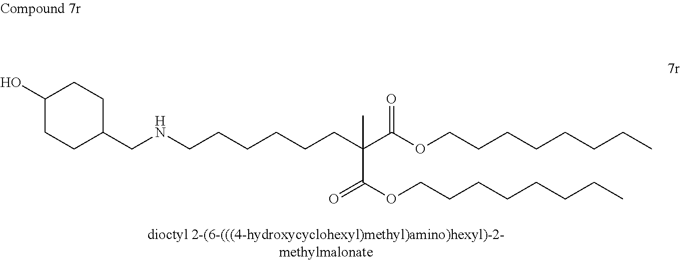 [SMILES: CCCCCCCCOC(=O)C(C)(CCCCCCNCC1CCC(O)CC1)C(=O)OCCCCCCCC]

Compound 7r was synthesized according to the general step 7 procedure in scheme 1. The title compound was obtained as a yellow oil. ¹H NMR (600 MHz, CDCl₃) δ 4.04-4.02 (m, 4H), 3.91 (br. s, 0.5H), 3.49-3.45 (m, 0.5H), 2.55-2.50 (m, 2H), 2.46-2.45 (m, 1H), 2.39-2.38 (m, 1H), 1.93-1.91 (m, 1H), 1.79-1.75 (m, 3H), 1.67-1.65 (m, 1H), 1.59-1.35 (m, 12H), 1.33 (s, 3H), 1.29-1.15 (m, 26H), 0.83-0.81 (m, 6H). ¹³C NMR (150 MHz, CDCl₃) δ 172.5, 70.7, 66.6, 65.2, 55.8, 55.3, 53.7, 50.0, 49.9, 36.8, 36.3, 35.5, 35.2, 32.1, 31.7, 29.8, 29.7, 29.5, 29.4, 29.2, 28.5, 27.1, 25.8, 25.1, 24.3, 22.6, 19.9, 14.0. MS (ESI): m/z [M+H]⁺ 554.4775 for C₃₃H₆₄NO₅.

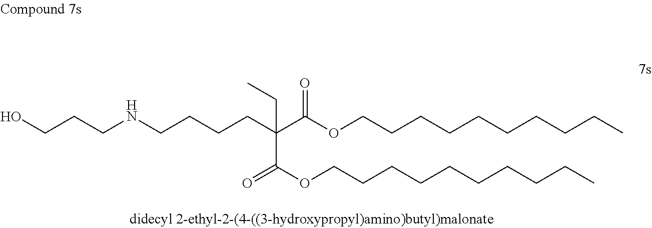 [SMILES: CCCCCCCCCCOC(=O)C(CC)(CCCCNCCCO)C(=O)OCCCCCCCCCC]

Compound 7s was synthesized according to the general step 7 procedure in scheme 1. The title compound was obtained as a yellow oil. ¹H NMR (600 MHz, CDCl₃) δ 4.09-4.05 (m, 4H), 3.78-3.77 (m, 2H), 2.87-2.85 (m, 2H), 2.61 (t, J=7.2 Hz, 2H), 1.90 (q, J=7.8 Hz, 2H), 1.86-1.83 (m, 2H), 1.70-1.68 (m, 2H), 1.59-1.55 (m, 4H), 1.50-1.48 (m, 2H), 1.27-1.15 (m, 30H), 0.87-0.84 (m, 6H), 0.79 (t, J=7.8 Hz, 3H). ¹³C NMR (150 MHz, CDCl₃) δ 171.9, 65.3, 64.0, 58.0, 49.8, 49.4, 31.9, 31.6, 30.5, 29.9, 29.6 (×2), 29.4, 29.3, 28.6, 25.9, 25.4, 22.7, 21.8, 14.1, 8.5. MS (ESI): m/z [M+H]⁺ 542.4770 for C₃₂H₆₄NO₅.

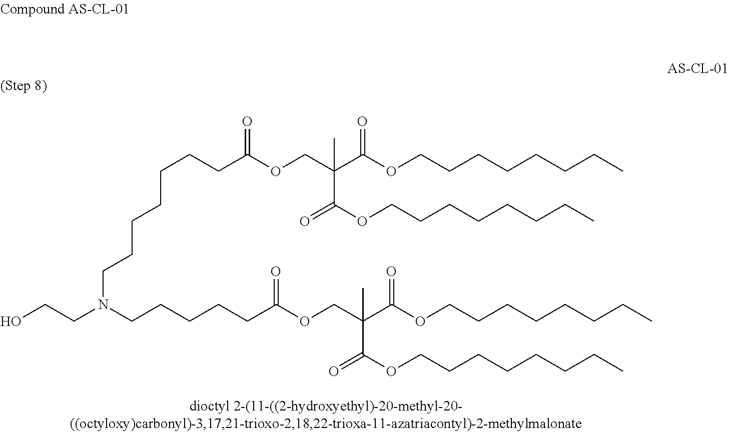 [SMILES: CCCCCCCCOC(=O)C(C)(COC(=O)CCCCCCCN(CCO)CCCCCC(=O)OCC(C)(C(=O)OCCCCCCCC)C(=O)OCCCCCCCC)C(=O)OCCCCCCCC]

A mixture solution of compound 6 (86 mg, 0.15 mmol) and KI (26 mg, 0.16 mmol) in MeCN/DCM was stirred at R.T, and then the addition of compound 7 (75 mg, 0.14 mmol) and K₂CO₃ (78 mg, 0.57 mmol). After stirring at 30′C overnight, the solvent was removed and the residue was dissolved in DCM. The organic solvent was washed with water and brine and dried over MgSO₄. The crude product was purified by silica gel column chromatography with 5% MeOH/1% NH₄OH in DCM to yield the compound AS-CL-01 (60 mg, 0.058 mmol, 39%/o) as a light-yellow oil. ¹H NMR (600 MHz, CDCl₃) δ 4.43 (s, 2H), 4.12 (t, J=6.6 Hz, 4H), 3.50 (t, J=5.4 Hz, 2H), 2.55 (t, J=5.4 Hz, 2H), 2.44-2.40 (m, 4H), 2.29-2.26 (m, 4H), 1.62-1.58 (m, 12H), 1.47 (s, 6H), 1.44-1.38 (m, 4H), 1.29-1.25 (m, 48H), 0.87 (t, J=7.2 Hz, 12H). ¹³C NMR (150 MHz, CDCl₃) δ 173.2, 173.0, 170.0(×2), 66.3, 65.9(×2), 58.4, 55.5, 53.9(×2), 53.8, 53.7(×2), 34.1, 34.0, 31.8(×2), 29.3, 29.2(×4), 29.1, 28.5(×2), 27.3, 27.2, 27.0, 26.9, 25.8(×2), 24.9, 24.8, 22.7(×2), 18.3(×2), 14.1(×2). MS (ESI): m/z [M+H]⁺ 1026.7822 for C₅₈H₁₀₈NO₁₃.

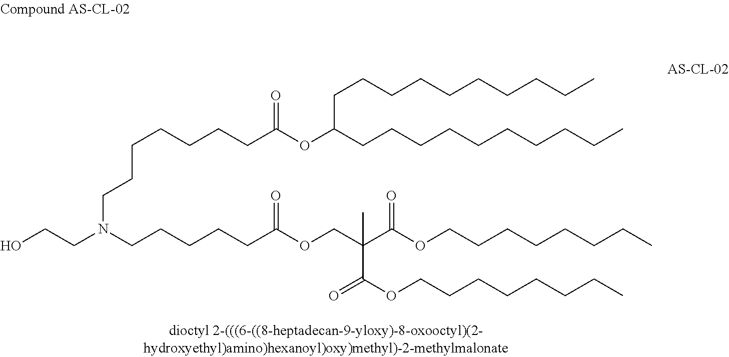 [SMILES: CCCCCCCCCCC(CCCCCCCCCC)OC(=O)CCCCCCCN(CCO)CCCCCC(=O)OCC(C)(C(=O)OCCCCCCCC)C(=O)OCCCCCCCC]

Compound AS-CL-02 was synthesized according to the general step 8 procedure in scheme 2. The title compound was obtained as a colorless oil. ¹H NMR (600 MHz, CDCl₃) δ ¹H NMR (600 MHz, CDCl₃) δ 4.84-4.81 (m, 1H), 4.42 (s, 2H), 4.10 (t, J=6.6 Hz, 4H), 3.48 (t, J=5.4 Hz, 2H), 2.53 (t, J=5.4 Hz, 2H), 2.40 ((t, J=7.2 Hz, 2H), 2.27-2.23 (m, 4H), 1.61 (m, 8H), 1.48-1.45 (m, 4H), 1.45 (s, 3H), 1.42-1.37 (m, 6H), 1.28-1.22 (m, 54H), 0.86-0.83 (m, 12H). ¹³C NMR (150 MHz, CDCl3) δ 173.6, 173.1, 169.9, 66.2, 65.8 (×2), 58.3, 55.5, 53.9, 53.8 (×2), 34.7, 34.1, 34.0, 31.9, 31.8, 29.52, 29.50, 29.26, 29.22 (×2), 29.17 (×2), 29.16 (×2), 29.09, 28.4, 27.3, 27.2, 27.1, 25.8, 25.3, 25.1, 24.8, 22.62, 22.61, 18.3, 14.1 (×2). MS (ESI): m/z [M+H]⁺ 938.8015 for C₅₆H₁₀₈NO₉.

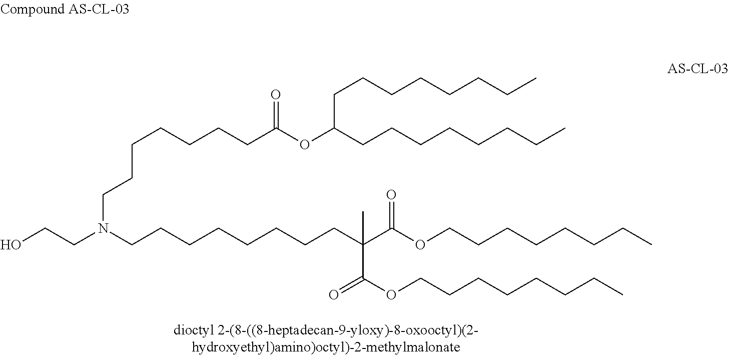 [SMILES: CCCCCCCCOC(=O)C(C)(CCCCCCCCN(CCO)CCCCCCCC(=O)OC(CCCCCCCC)CCCCCCCC)C(=O)OCCCCCCCC]

Compound AS-CL-03 was synthesized according to the general step 8 procedure in scheme 2. The title compound was obtained as a colorless oil. (600 MHz, CDCl₃) δ ¹H NMR (600 MHz, CDCl₃) δ 4.86-4.82 (m, 1H), 4.10-4.04 (m, 4H), 3.49 (t, J=5.4 Hz, 2H), 2.54 (t, J=5.4 Hz, 2H), 2.41-2.39 (m, 4H), 2.25 (t, J=7.2 Hz, 2H), 1.82-1.80 (m, 2H), 1.60-1.56 (m, 6H), 1.48-1.47 (m, 4H), 1.43-1.38 (m, 6H), 1.37 (s, 3H), 1.36-1.23 (m, 58H), 0.86-0.84 (m, 12H). ¹³C NMR (150 MHz, CDCl₃) δ 173.6, 172.6, 74.1, 65.3, 58.4, 55.5, 53.9, 53.87, 53.8, 35.6, 34.7, 34.2, 31.9, 31.8, 29.9, 29.6, 29.5, 29.4, 29.3, 29.27 (×2), 29.24 (×2), 29.2, 28.6, 27.5, 27.3, 27.22, 27.2, 25.9, 25.4, 25.2, 24.4, 22.7, 22.67, 19.9, 14.14, 14.11. MS (ESI): m/z [M+H]⁺ 894.8114 for C₅₅H₁₀₈NO₇.

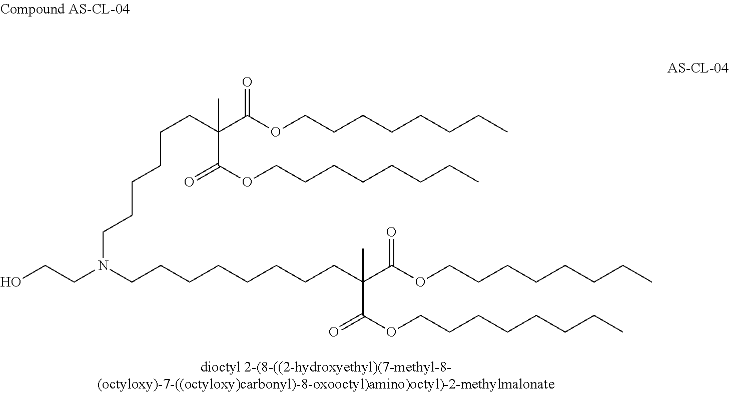 [SMILES: CCCCCCCCOC(=O)C(C)(CCCCCCCCN(CCO)CCCCCCC(C)(C(=O)OCCCCCCCC)C(=O)OCCCCCCCC)C(=O)OCCCCCCCC]

Compound AS-CL-04 was synthesized according to the general step 8 procedure in scheme 2. The title compound was obtained as a colorless oil. (600 MHz, CDCl₃) δ ¹H NMR (600 MHz, CDCl₃) δ 4.06-4.05 (m, 4H), 3.47 (t, J=4.8 Hz, 2H), 2.52 (t, J=4.8 Hz, 2H), 2.40-2.37 (m, 4H), 1.81-1.79 (m, 4H), 1.58-1.54 (m, 6H), 1.37 (s, 6H), 1.35-1.24 (m, 62H), 0.85-0.83 (m, 12H). ¹³C NMR (150 MHz, CDCl₃) δ 172.57, 172.53, 65.26, 65.24, 58.4, 55.5, 53.9, 53.8, 53.77, 53.7, 35.58, 35.55, 31.8 (×2), 29.91, 29.89, 29.5, 29.4, 29.21, 29.18, 28.5(×2), 27.5, 27.3, 27.2, 27.1, 25.9 (×2), 24.4, 24.3, 22.6 (×2), 19.9 (×2), 14.1 (×2). MS (ESI): m/z [M+H]⁺ 938.8026 for C₅₆H₁₀₈NO₉.

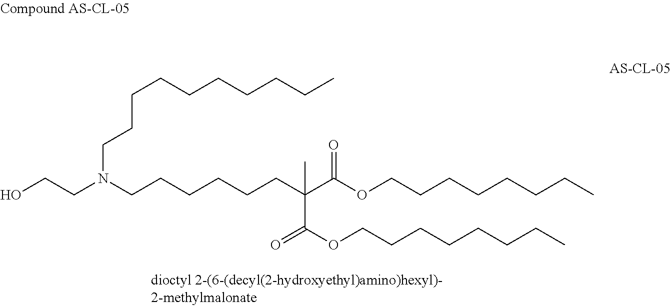 [SMILES: CCCCCCCCCCN(CCO)CCCCCCC(C)(C(=O)OCCCCCCCC)C(=O)OCCCCCCCC]

Compound AS-CL-0S was synthesized according to the general step 8 procedure in scheme 2. The title compound was obtained as a colorless oil. ¹H NMR (600 MHz, CDCl₃) δ 4.09-4.07 (m, 4H), 3.50 (t, J=5.4 Hz, 2H), 2.55 (t, J=5.4 Hz, 2H), 2.42-2.40 (m, 4H), 1.84-1.81 (m, 2H), 1.61-1.57 (m, 4H), 1.43-1.39 (m, 4H), 1.38 (s, 3H), 1.29-1.25 (m, 40H), 0.88-0.85 (m, 9H). ¹³C NMR (150 MHz, CDCl₃) δ 172.6, 65.4, 58.4, 55.5, 53.9, 53.84, 53.82, 35.6, 32.0, 31.9, 30.0, 29.7, 29.6, 29.4, 29.3 (×2), 29.2 (×2), 28.6, 27.5, 27.3, 27.2, 27.1, 25.9, 24.4, 22.74, 22.7, 20.0, 14.17, 14.14. MS (ESI): m/z [M+H]⁺ 626.5725 for C₃₈H₇₆NO₅.

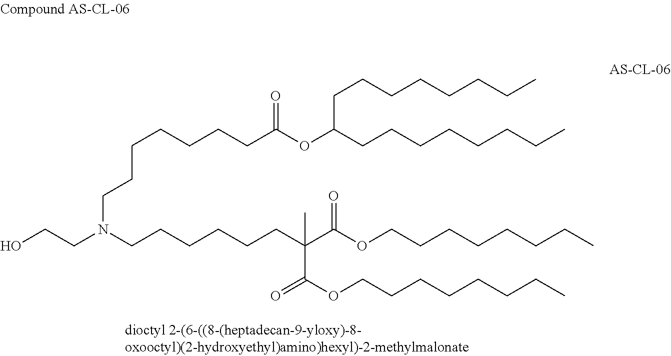 [SMILES: CCCCCCCCOC(=O)C(C)(CCCCCCN(CCO)CCCCCCCC(=O)OC(CCCCCCCC)CCCCCCCC)C(=O)OCCCCCCCC]

Compound AS-CL-06 was synthesized according to the general step 8 procedure in scheme 2. The title compound was obtained as a colorless oil. ¹H NMR (600 MHz, CDCl₃) δ 4.85-4.83 (m, 1H), 4.07-4.05 (m, 4H), 3.48 (t, J=5.4 Hz, 2H), 2.53 (t, J=5.4 Hz, 2H), 2.41-2.38 (m, 4H), 2.25 (t, J=7.2 Hz, 2H), 1.82-1.80 (m, 2H), 1.59-1.55 (m, 6H), 1.48-1.46 (m, 4H), 1.39-1.36 (m, 4H), 1.36 (s, 3H), 1.25-1.23 (m, 56H), 0.86-0.84 (m, 12H). ¹³C NMR (150 MHz, CDCl3) δ 173.6, 172.6, 74.1, 65.3, 58.4, 55.5, 53.9 (×2), 53.8, 35.6, 34.7, 34.2, 31.9, 31.8, 30.0, 29.6, 29.5, 29.3, 29.27 (×2), 29.24 (×2), 28.6, 27.4, 27.3, 27.2, 27.1, 25.9, 25.4, 25.2, 24.4, 22.70, 22.68, 19.9, 14.14, 14.12. MS (ESI): m/z [M+H]⁺ 866.7800 for C₅₃H₁₀₄NO₇.

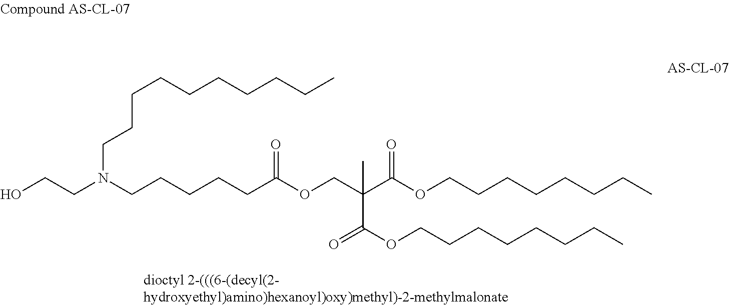 [SMILES: CCCCCCCCCCN(CCO)CCCCCC(=O)OCC(C)(C(=O)OCCCCCCCC)C(=O)OCCCCCCCC]

Compound AS-CL-07 was synthesized according to the general step 8 procedure in scheme 2. The title compound was obtained as a colorless oil. ¹H NMR (600 MHz, CDCl₃) δ ¹H NMR (600 MHz, CDCl3) δ 4.43 (s, 2H), 4.11 (t, J=6.6 Hz, 4H), 3.50 (t, J=5.4 Hz, 2H), 2.55. (t, J=4.8 Hz, 2H), 2.43-2.40 (m, 4H), 2.28 (t, J=7.2 Hz, 2H), 1.61-1.59 (m, 6H), 1.47 (s, 3H), 1.44-1.40 (m, 4H), 1.27-1.24 (m, 36H), 0.88-0.85 (m, 9H). ¹³C NMR (150 MHz, CDCl₃) δ 173.0, 170.0, 66.3, 65.9, 58.4, 55.5, 53.9, 53.7, 34.0, 31.9, 31.8, 29.7 (×2), 29.6, 29.4 (×2), 29.2, 28.5, 27.5, 27.2, 27.0, 26.9, 25.8, 24.8, 22.7, 22.6, 18.3, 14.15, 14.12. MS (ESI): m/z [M+H]⁺ 670.5623 for C₃₉H₇₆NO₇.

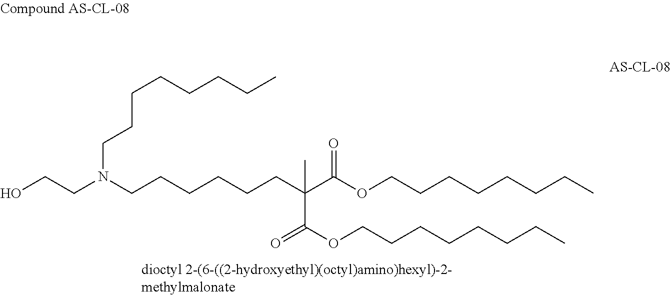 [SMILES: CCCCCCCCOC(=O)C(C)(CCCCCCN(CCO)CCCCCCCC)C(=O)OCCCCCCCC]

Compound AS-CL-08 was synthesized according to the general step 8 procedure in scheme 2. The title compound was obtained as a colorless oil. ¹H NMR (600 MHz, CDCl₃) δ 4.09-4.07 (m, 4H), 3.50 (t, J=5.4 Hz, 2H), 2.55 (t, J=5.4 Hz, 2H), 2.42-2.40 (m, 4H), 1.84-1.81 (m, 2H), 1.60-1.57 (m, 4H), 1.40-1.37 (m, 4H), 1.37 (s, 3H), 1.29-1.20 (m, 36H), 0.88-0.85 (m, 9H). ¹³C NMR (150 MHz, CDCl₃) δ 172.6, 65.3, 58.4, 55.5, 53.9, 53.84, 53.82, 35.6, 31.9, 31.8, 29.9, 29.6, 29.4, 29.3, 29.2, 28.6, 27.5, 27.3, 27.2, 27.1, 25.9, 24.4, 22.7, 22.6, 19.9, 14.15, 14.13. MS (ESI): m/z [M+H]⁺ 598.5402 for C₃₋₆H₇₂NO₅.

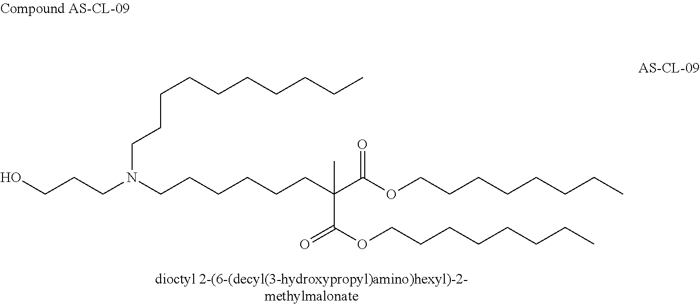 [SMILES: CCCCCCCCCCN(CCCO)CCCCCCC(C)(C(=O)OCCCCCCCC)C(=O)OCCCCCCCC]

Compound AS-CL-09 was synthesized according to the general step 8 procedure in scheme 2. The title compound was obtained as a colorless oil. ¹H NMR (600 MHz, CDCl₃) δ 4.07 (t, J=6.6 Hz, 4H), 3.77 (t, J=4.8 Hz, 2H), 2.61 (t, J=5.4 Hz, 2H), 2.37 (t, J=7.2 Hz, 2H), 1.83-1.81 (m, 2H), 1.67-1.63 (m, 2H), 1.61-1.56 (m, 4H), 1.46-1.42 (m, 4H), 1.37 (s, 3H), 1.28-1.19 (m, 40H), 0.87-0.85 (m, 9H). ¹³C NMR (150 MHz, CDCl₃) δ 172.6, 65.3, 64.9, 55.4, 54.3, 54.2, 53.8, 35.6, 31.9, 31.8, 30.0, 29.7, 29.4, 29.3 (×2), 29.2 (×2), 28.6, 27.8, 27.6, 27.4, 26.9, 26.8, 25.9, 24.4, 22.8, 22.7, 19.9, 14.2, 14.1. MS (ESI): m/z [M+H]⁺ 640.5868 for C₃₉H₇₈NO₅.

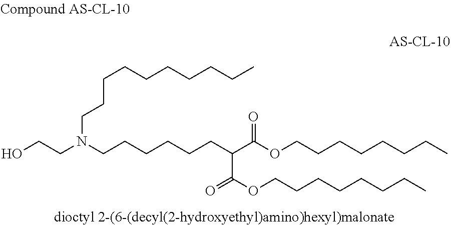 [SMILES: CCCCCCCCCCN(CCO)CCCCCCC(C(=O)OCCCCCCCC)C(=O)OCCCCCCCC]

Compound AS-CL-10 was synthesized according to the general step 8 procedure in scheme 2. The title compound was obtained as a colorless oil. ¹H NMR (600 MHz, CDCl₃) δ 4.12-4.05 (m, 4H), 3.49 (t, J=5.4 Hz, 2H), 3.28 (t, J=7.8 Hz, 1H), 2.53 (t, J=5.4 Hz, 2H), 2.40 (t, J=7.2 Hz, 4H), 1.87-1.84 (m, 2H), 1.62-1.57 (m, 4H), 1.42-1.36 (m, 4H), 1.29-1.23 (m, 40H), 0.86-0.84 (m, 9H). ¹³C NMR (150 MHz, CDCl3) δ 169.7, 65.5, 58.4, 55.5, 53.9, 53.8, 52.1, 32.0, 31.8, 29.7, 29.6 (×2), 29.4, 29.23 (×2), 29.22, 28.7, 28.6, 27.5, 27.4, 27.2 (×2), 27.1, 25.9, 22.7, 22.6, 14.15, 14.12. MS (ESI): m/z [M+H]⁺ 612.5555 for C₃₇H₇₄NO₅.

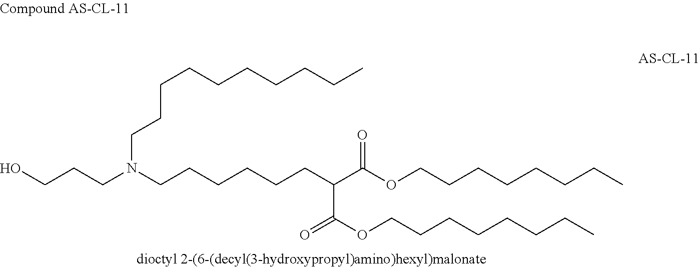 [SMILES: CCCCCCCCCCN(CCCO)CCCCCCC(C(=O)OCCCCCCCC)C(=O)OCCCCCCCC]

Compound AS-CL-11 was synthesized according to the general step 8 procedure in scheme 2. The title compound was obtained as a colorless oil. ¹H NMR (600 MHz, CDCl₃) δ 4.11-4.05 (m, 4H), 3.74 (t, J=5.4 Hz, 2H), 3.27 (t, J=7.8 Hz, 1H), 2.59-2.58 (m, 2H), 2.36-2.34 (m, 4H), 1.86-1.81 (m, 2H), 1.63-1.57 (m, 6H), 1.43-1.39 (m, 4H), 1.31-1.20 (m, 40H), 0.85-0.83 (m, 9H). ¹³C NMR (150 MHz, CDCl₃) δ 169.6, 65.4, 64.8, 55.3, 54.2, 54.1, 52.1, 31.9, 31.8, 29.62(×2), 29.60 (×2), 29.3, 29.2(×2), 28.7, 28.5, 27.8, 27.5, 27.3, 27.2, 26.8, 26.7, 25.8, 22.7, 22.6, 14.09, 14.07. MS (ESI): m/z [M+H]⁺ 626.5719 for C₃₈H₇₆NO₅.

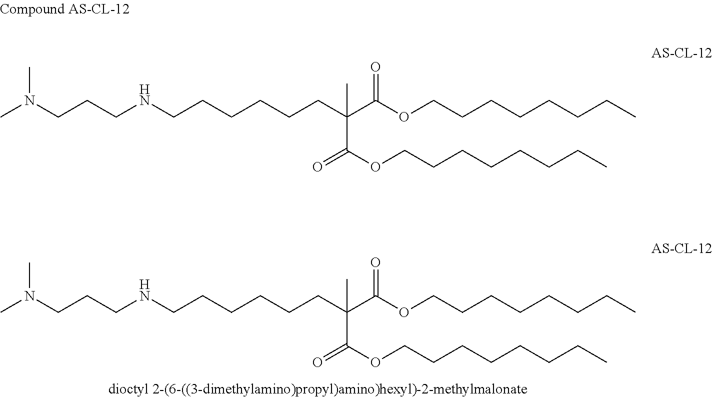

Compound AS-CL-12 was synthesized according to the general step 7 procedure in scheme 2. The title compound was obtained as a colorless oil. ¹H NMR (600 MHz, CDCl₃) δ 4.06 (t, J=6.6 Hz, 4H), 2.81 (t, J=6.6 Hz, 2H), 2.67 (t, J=7.2 Hz, 2H), 2.41 (t, J=6.6 Hz, 2H), 2.24 (s, 6H), 1.82-1.75 (m, 4H), 1.59-1.55 (m, 6H), 1.36 (s, 3H), 1.31-1.24 (m, 26H), 0.85 (t, J=7.2 Hz, 6H). ¹³C NMR (150 MHz, CDCl₃) δ 172.5, 65.3, 58.8, 53.7, 49.1 (×2), 45.5, 35.5, 31.8, 29.6, 29.2 (×2), 28.7, 28.5, 26.9, 25.9, 25.6, 24.2, 22.7, 19.9, 14.1. MS (ESI): m/z [M+H]⁺ 527.4779 for C₃₁H₆₃N₂O₄.

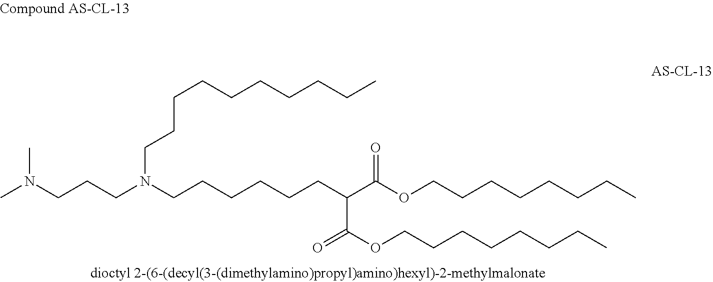 [SMILES: CCCCCCCCCCN(CCCCCCC(C(=O)OCCCCCCCC)C(=O)OCCCCCCCC)CCCN(C)C]

Compound AS-CL-13 was synthesized according to the general step 8 procedure in scheme 2. The title compound was obtained as a colorless oil. ¹H NMR (600 MHz, CDCl₃) δ 4.06-4.04 (m, 4H), 3.62-3.60 (m, 2H), 3.49-3.45 (m, 2H), 3.34 (s, 6H), 2.72-2.70 (m, 2H), 2.54-2.52 (m, 2H), 1.97-1.92 (m, 2H), 1.81-1.77 (m, 2H), 1.72-1.68 (m, 2H), 1.59-1.53 (m, 4H), 1.45-1.42 (m, 2H), 1.35 (s, 3H), 1.35-1.20 (m, 40H), 0.85-0.83 (m, 9H). ¹³C NMR (150 MHz, CDCl3) δ 172.5, 65.3, 64.4, 62.8, 53.7, 51.7, 49.9, 46.0, 35.5, 31.8, 31.7, 29.9, 29.8, 29.37, 29.35, 29.2, 29.13 (×2), 29.12 (×2), 28.5, 27.1, 26.2, 25.8, 24.2, 23.5, 22.8, 22.6, 19.8, 14.0 (×2). MS (ESI): m/z [M+H]⁺ 667.6340 for C₄₁H₈₃N₂O₄.

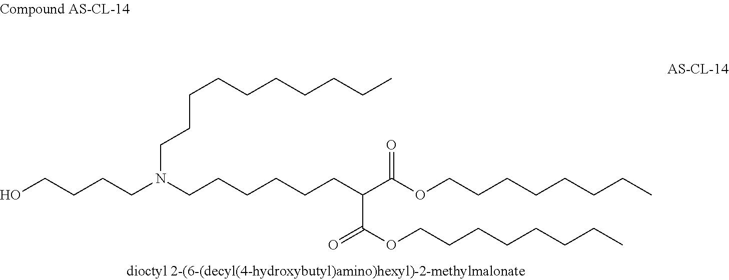 [SMILES: CCCCCCCCCCN(CCCCO)CCCCCCC(C(=O)OCCCCCCCC)C(=O)OCCCCCCCC]

Compound AS-CL-14 was synthesized according to the general step 8 procedure in scheme 2. The title compound was obtained as a colorless oil. ¹H NMR (600 MHz, CDCl₃) δ 4.06 (t, J=6.6 Hz, 4H), 3.52-3.51 (m, 2H), 2.42-2.39 (m, 6H), 1.82-1.79 (m, 2H), 1.62-1.54 (m, 8H), 1.46-1.42 (m, 4H), 1.35 (s, 3H), 1.26-1.16 (m, 40H), 0.86-0.83 (m, 9H). ¹³C NMR (150 MHz, CDCl₃) δ 172.6, 65.3, 62.6, 54.6, 53.8, 53.7, 53.6, 35.6, 32.7, 31.9, 31.8, 29.9, 29.7, 29.6, 29.5, 29.3, 29.2(×2), 28.5, 27.7, 27.5, 26.2, 25.9, 25.8, 25.7, 24.4, 22.7, 22.6, 19.9, 14.13, 14.11. MS (ESI): m/z [M+H]⁺ 654.6039 for C₄₀H₈₀NO₅.

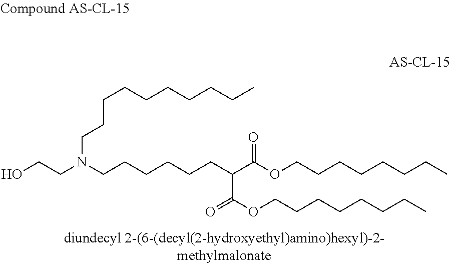 [SMILES: CCCCCCCCCCN(CCO)CCCCCCC(C(=O)OCCCCCCCC)C(=O)OCCCCCCCC]

Compound AS-CL-15 was synthesized according to the general step 8 procedure in scheme 2. The title compound was obtained as a colorless oil. ¹H NMR (600 MHz, CDCl₃) δ 4.08 (t, J=6.6 Hz, 4H), 3.50 (t, J=5.4 Hz, 2H), 2.55 (t, J=5.4 Hz, 2H), 2.41 (t, J=7.2 Hz, 4H), 1.84-1.81 (m, 2H), 1.60-1.57 (m, 4H), 1.41-1.38 (m, 4H), 1.37 (s, 3H), 1.29-1.20 (m, 52H), 0.88-0.85 (m, 9H). ¹³C NMR (150 MHz, CDCl₃) δ 172.6, 65.3, 58.4, 55.5, 53.9, 53.84, 53.80, 35.6, 32.0, 29.9, 29.7, 29.65, 29.60, 29.4, 29.3, 28.6, 27.5, 27.3, 27.2, 27.1, 25.9, 24.4, 22.7, 19.9, 14.2. MS (ESI): m/z [M+H]⁺ 710.6655 for C₄₄H₈₈NO₅.

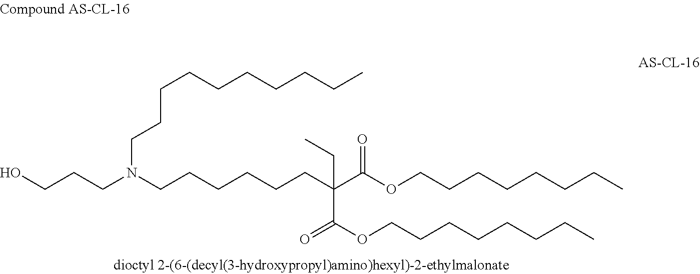 [SMILES: CCCCCCCCCCN(CCCO)CCCCCCC(CC)(C(=O)OCCCCCCCC)C(=O)OCCCCCCCC]

Compound AS-CL-16 was synthesized according to the general step 8 procedure in scheme 2. The title compound was obtained as a colorless oil. ¹H NMR (600 MHz, CDCl₃) δ 4.08 (t, J=6.6 Hz, 4H), 3.77 (L J=4.8 Hz, 2H), 2.62 (t, J=4.8 Hz, 2H), 2.39 (t, J=7.2 Hz, 4H), 1.90 (q, J=7.2 Hz, 2H), 1.85-1.82 (m, 2H), 1.67-1.65 (m, 2H), 1.61-1.56 (m, 4H), 1.46-1.42 (m, 4H), 1.30-1.24 (m, 38H), 1.15-1.11 (m, 2H), 0.88-0.86 (m, 9H), 0.79 (t, J=7.2 Hz, 3H). ¹³C NMR (150 MHz, CDCl₃) δ 172.0, 65.2, 64.8, 58.1, 55.3, 54.2, 54.1, 32.0, 31.8, 31.7, 29.9, 29.7, 29.6, 29.4, 29.3, 29.2 (×2), 28.6, 27.8, 27.6, 27.4, 26.8, 26.7, 25.9, 25.2, 24.0, 22.73, 22.69, 14.2, 14.1, 8.5. MS (ESI): m/z [M+H]⁺ 654.6036 for C₄₀H₈₀NO₅.

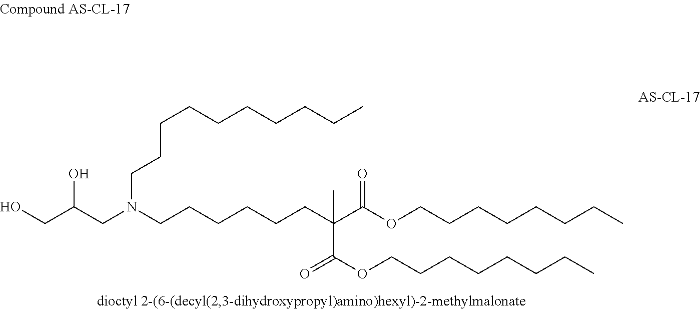 [SMILES: CCCCCCCCCCN(CCCCCCC(C)(C(=O)OCCCCCCCC)C(=O)OCCCCCCCC)CC(O)CO]

Compound AS-CL-17 was synthesized according to the general step 8 procedure in scheme 2. The title compound was obtained as a colorless oil. ¹H NMR (600 MHz, CDCl₃) δ 4.08 (t, J=6.6 Hz, 4H), 3.73-3.68 (m, 2H), 3.48-3.45 (m, 1H), 2.57-2.54 (m, 1H), 2.51-2.46 (m, 2H), 2.41-2.37 (m, 3H), 1.84-1.81 (m, 2H), 1.61-1.57 (m, 4H), 1.48-1.38 (m, 4H), 1.37 (s, 3H), 1.32-1.18 (m, 40H), 0.88-0.85 (m, 9H). ¹³C NMR (150 MHz, CDCl₃) δ 172.6, 67.3, 65.3, 64.9, 56.8, 54.4, 54.3, 53.8, 35.6, 31.9, 31.8, 29.9, 29.7, 29.6, 29.3, 29.22 (×2), 29.20, 28.5, 27.5, 27.2, 27.1, 27.0, 25.9, 24.3, 22.7, 22.6, 19.9, 14.12, 12.10. MS (ESI): m/z [M+H]⁺ 656.5822 for C₃₉H₇₈NO₆.

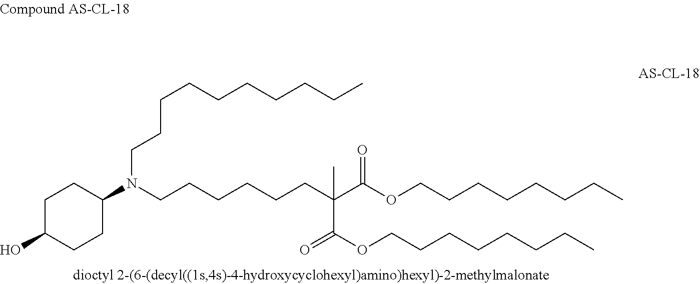 [SMILES: CCCCCCCCCCN(CCCCCCC(C)(C(=O)OCCCCCCCC)C(=O)OCCCCCCCC)[C@H]1CC[C@@H](O)CC1]

Compound AS-CL-18 was synthesized according to the general step 8 procedure in scheme 2. The title compound was obtained as a colorless oil. ¹H NMR (600 MHz, CDCl3) δ 4.07 (t, J=6.6 Hz, 4H), 3.97 (br. s, 1H), 2.53-2.46 (m, 5H), 1.83-1.80 (m, 4H), 1.68-1.56 (m, 8H), 1.51-1.41 (m, 6H), 1.37 (s, 3H), 1.31-1.17 (m, 40H), 0.87-0.85 (m, 9H). MS (ESI): m/z [M+H]⁺ 680.6183 for C₄₂H₈₂NO₅.

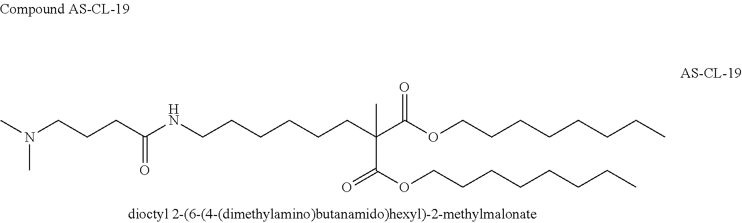 [SMILES: CCCCCCCCOC(=O)C(C)(CCCCCCNC(=O)CCCN(C)C)C(=O)OCCCCCCCC]

Compound AS-CL-19 was synthesized according to the procedure in scheme 3. A mixture solution of compound 6b (0.37 g, 0.67 mmol) and potassium phthalimide (0.62 g, 3.35 mmol) in DMF was stirred at R.T for 3 hours. After filtration and evaporation, the residue was purified by silica gel column chromatography with EA/Hex (1/10) to yield the compound 6b-1 (311 mg, 84%). ¹H NMR (600 MHz, CDCl₃) δ 7.84-7.82 (m, 2H), 7.71-7.69 (m, 2H), 4.08 (t, 4H), 3.67 (t, 2H), 1.84-1.81 (m, 2H), 1.66-1.64 (m, 2H), 1.61-1.57 (m, 4H), 1.37 (s, 3H), 1.34-1.25 (m, 26H), 0.87 (t, 6H). ¹³C NMR (150 MHz, CDCl₃) δ 172.5, 168.4, 133.8, 132.1, 123.1, 65.3, 53.7, 37.9, 35.5, 31.7, 29.5, 29.2, 28.5, 28.4, 26.7, 26.2, 25.8, 24.2, 22.6, 19.8, 14.0.

A mixture solution of compound 6b-1 (311 mg, 0.544 mmol) and hydrazine (50 mg, 1.63 mmol) in MeOH was stirred under reflux for 2 hours. After evaporation, the residue was dissolved in DCM and filtration. The crude compound was purified by silica gel column chromatography with 10% MeOH/DCM/1% NH₄OH to yield the compound 6b-2 (160 mg, 66%). ¹H NMR (600 MHz, CDCl3) δ 4.08 (t, 4H), 2.67 (t, 2H), 1.85-1.82 (m, 2H), 1.62-1.57 (m, 4H), 1.43-1.41 (m, 2H), 1.38 (s, 3H), 1.31-1.25 (m, 26H), 0.87 (t, 6H). ¹³C NMR (150 MHz, CDCl₃) δ 172.6, 65.3, 53.8, 42.1, 35.5, 33.7, 31.9, 29.8, 29.6, 29.5, 29.3, 29.2, 28.5, 26.7, 25.9, 24.3, 22.7, 19.9, 14.1.

A mixture solution of 4-(dimethylamino)butanoic acid (0.071 g, 0.426 mmol), SOCl₂ (0.101 g, 0.852 mmol) and 2 drops of DMF in DCM was stirred at 0° C. overnight. After evaporation, the residue was reacted with compound 6b-2 in DCM at 0° C., and Et₃N was added. The reaction was monitored by TLC. After evaporation, the residue was purified by silica gel column chromatography with 10% MeOH/DCM/l % NH₄OH to yield the compound AS-CL-19 (76 mg, 45%) as a yellow oil. ¹H NMR (600 MHz, CDCl₃) δ 6.41 (br. s, 1H), 4.08 (t, J=6.6 Hz, 4H), 3.20 (q, J=6.6 Hz, 2H), 2.31 (t, J=7.2 Hz, 2H), 2.24 (4, J=7.2 Hz, 2H), 2.21 (s, 6H), 1.84-1.82 (m, 2H), 1.81-1.76 (m, 2H), 1.61-1.58 (m, 4H), 1.47-1.45 (m, 2H), 1.38 (s, 3H), 1.31-2.20 (m, 26H), 0.87 (t, J=7.2 Hz, 6H). ¹³C NMR (150 MHz, CDCl₃) 172.7, 172.4, 65.2, 58.7, 53.6, 45.1, 39.3, 35.4, 34.7, 31.7, 29.5, 29.1, 29.0, 28.4, 26.6, 25.7, 24.1, 23.1, 22.5, 19.8, 14.0. MS (ESI): m/z [M+H]⁺ 555.4727 for C₃₂H₄₃N₂O₅.

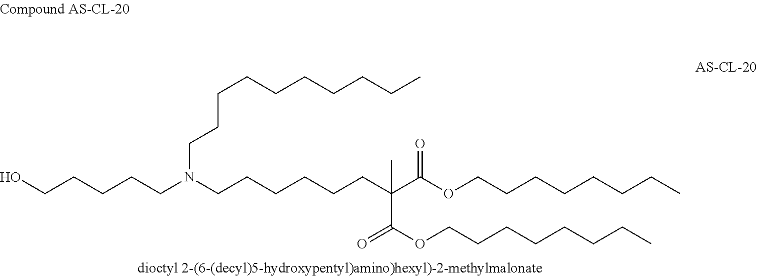 [SMILES: CCCCCCCCCCN(CCCCCO)CCCCCCC(C)(C(=O)OCCCCCCCC)C(=O)OCCCCCCCC]

Compound AS-CL-20 was synthesized according to the general step 8 procedure in scheme 2. The title compound was obtained as a colorless oil. ¹H NMR (600 MHz, CDCl₃) δ 4.07 (t, J=6.6 Hz, 4H), 3.63-3.61 (m, 2H), 2.52-2.46 (m, 6H), 1.83-1.80 (m, 2H), 1.60-1.46 (m, 12H), 1.40-1.34 (m, 5H), 1.31-1.18 (m, 40H), 0.87-0.85 (m, 9H). ¹³C NMR (150 MHz, CDCl₃) δ 172.6, 65.4, 62.6, 53.9, 53.8, 53.7, 35.6, 32.4, 31.9, 31.8, 29.8, 29.7, 29.6, 29.5, 29.4, 29.3, 29.2, 28.6, 27.6, 27.3, 26.1, 26.0 (×2), 25.9, 24.3, 23.7, 22.72, 22.68, 19.9, 14.2, 14.1. MS (ESI): m/z [M+H]⁺ 668.6194 for C₄₁H₅₂NO₅.

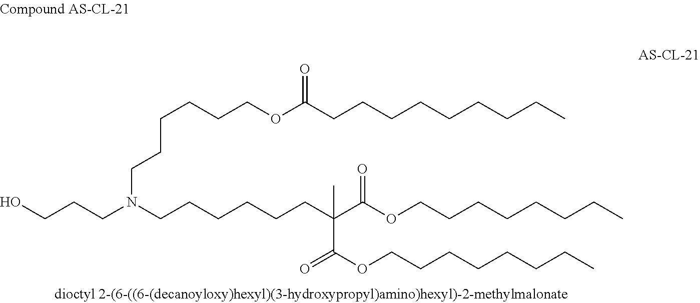 [SMILES: CCCCCCCCCC(=O)OCCCCCCN(CCCO)CCCCCCC(C)(C(=O)OCCCCCCCC)C(=O)OCCCCCCCC]

Compound AS-CL-21 was synthesized according to the general step 8 procedure in scheme 2. The title compound was obtained as a colorless oil. ¹H NMR (600 MHz, CDCl₃) δ 4.08 (t, J=6.6 Hz, 4H), 4.05 (t, J=6.6 Hz, 2H), 3.78 (t, J=5.4 Hz, 2H), 2.62 (t, J=5.4 Hz, 2H), 2.41-2.38 (m, 4H), 2.28 (t, J=7.2 Hz, 2H), 1.84-1.81 (m, 2H), 1.67-1.58 (m, 10H), 1.47-1.41 (m, 4H), 1.38 (s, 3H), 1.37-1.20 (m, 42H), 0.88-0.85 (m, 9H). ¹³C NMR (150 MHz, CDCl₃) δ 174.1, 172.6, 65.4, 64.8, 64.3, 55.3, 54.2, 54.1, 53.8, 35.6, 34.5, 31.9, 31.8, 30.0, 29.5, 29.3 (×2), 29.26 (×2), 29.24 (×2), 28.7, 28.6, 27.8, 27.4, 27.2, 26.8, 26.7, 26.0, 25.9, 25.1, 24.4, 22.72, 22.70, 19.9, 14.1. MS (ESI): m/z [M+H]⁺ 754.6549 for C₄₅H₈₈NO₇.

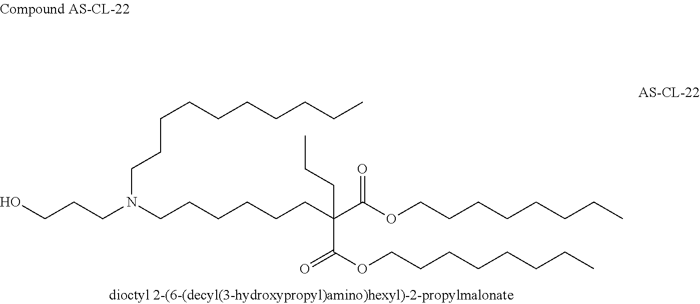 [SMILES: CCCCCCCCCCN(CCCO)CCCCCCC(CCC)(C(=O)OCCCCCCCC)C(=O)OCCCCCCCC]

Compound AS-CL-22 was synthesized according to the general step 8 procedure in scheme 2. The title compound was obtained as a colorless oil. ¹H NMR (600 MHz, CDCl₃) δ 4.08 (t, J=6.6 Hz, 4H), 3.78 (t, J=5.4 Hz, 2H), 2.61 (t, J=5.4 Hz, 2H), 2.38 (t, J=7.8 Hz, 4H), 1.86-1.82 (m, 4H), 1.68-1.63 (m, 2H), 1.61-1.57 (m, 4H), 1.47-1.41 (m, 4H), 1.32-1.22 (m, 38H), 1.18-1.11 (m, 4H), 0.91 (t, J=7.2 Hz, 3H), 0.88-0.86 (m, 9H). ¹³C NMR (150 MHz, CDCl₃) δ 172.1, 65.2, 64.9, 57.7, 55.4, 54.3, 54.2, 34.6, 32.4, 32.0, 31.9, 30.0, 29.70, 29.69, 29.67, 29.4, 29.3, 29.2, 28.6, 27.8, 27.6, 27.4, 26.9, 26.8, 26.0, 24.1, 22.8, 22.7, 17.5, 14.5, 14.18, 14.15. MS (ESI): m/z [M+H]⁺ 668.6180 for C₄₁H₈₂NO₅.

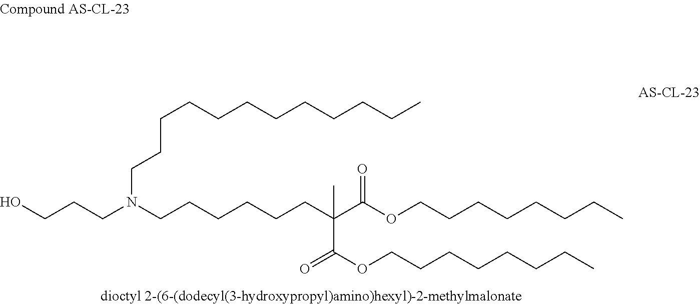 [SMILES: CCCCCCCCCCCCN(CCCO)CCCCCCC(C)(C(=O)OCCCCCCCC)C(=O)OCCCCCCCC]

Compound AS-CL-23 was synthesized according to the general step 8 procedure in scheme 2. The title compound was obtained as a colorless oil. ¹H NMR (600 MHz, CDCl₃) δ 4.08 (t, J=6.6 Hz, 4H), 3.78 (t, J=5.4 Hz, 2H), 2.61 (t, J=5.4 Hz, 2H), 2.39-2.37 (m, 4H), 1.84-1.81 (m, 2H), 1.67-1.64 (m, 2H), 1.62-1.57 (m, 4H), 1.47-1.42 (m, 4H), 1.38 (s, 3H), 1.32-1.20 (m, 44H), 0.88-0.86 (m, 9H). ¹³C NMR (150 MHz, CDCl₃) δ 172.6, 65.3, 64.9, 55.4, 54.3, 54.2, 53.8, 35.6, 32.0, 31.8, 29.9, 29.7 (×2), 29.4, 29.3 (×3), 29.2 (×2), 28.6, 27.8, 27.6, 27.4, 26.9, 26.8, 25.9, 24.4, 22.7, 22.6, 19.9, 14.2, 14.1. MS (ESI): m/z [M+H]⁺ 668.6181 for C₄₁H₅₂NO₅.

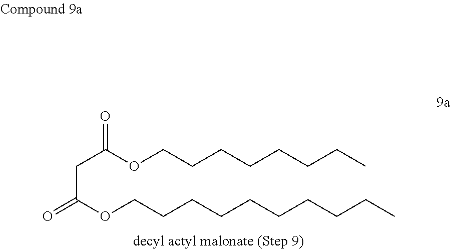 [SMILES: CCCCCCCCCCOC(=O)CC(=O)OCCCCCCCC]

A solution of Meldrum's acid (2.5 g, 17.35 mmol) and octanol (2.26 g, 17.35 mmol) in toluene was refluxed for 4 hours. After evaporation, the residue was dissolved in DCM, and then the addition of 1-decanol (4.12 g, 2.6 mmol), EDCI (5 g, 2.6 mmol) and DMAP (0.42 g, 3.47 mmol). The mixture solution was stirred at R.T for 4 hours. After washing with 2N HCl_{(aq)} and brine, the crude compound was purified by silica gel column chromatography with EA/Hex (1/50) to yield the desired compound 9a (5.65 g, 90%) as a colorless oil. ¹H NMR (600 MHz, CDCl₃) δ 4.11 (t, J=6.6 Hz, 4H), 3.34 (s, 2H), 1.64-1.59 (m, 4H), 1.32-1.24 (m, 24H), 0.87-0.85 (m, 6H). ¹³C NMR (150 MHz, CDCl₃) δ 166.7, 65.7, 41.7, 31.9, 31.8, 29.6, 29.3, 29.25, 29.20, 28.5, 25.8, 22.7, 22.6, 14.11, 14.08. MS (ESI): m/z [M+Na]⁺ 379.2823 for C₂₁H₄₀O₄Na.

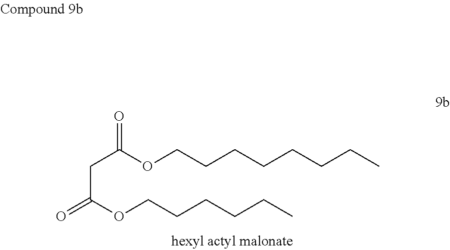 [SMILES: CCCCCCCCOC(=O)CC(=O)OCCCCCC]

Compound 9b was synthesized according to the step 9 procedure in scheme 4. The title compound was obtained as a colorless oil. ¹H NMR (600 MHz, CDCl₃) δ 4.10 (t, J=6.6 Hz, 4H), 3.33 (s, 2H), 1.61-1.59 (m, 4H), 1.31-1.26 (m, 16H), 0.85-0.84 (m, 6H). ¹³C NMR (150 MHz, CDCl₃) δ 166.7, 65.6, 41.7, 31.8, 31.4, 29.2 (×2), 28.5, 28.4, 25.8, 25.5, 22.6, 22.5, 14.1, 14.0. MS (ESI): m/z [M+Na]⁺ 323.2190 for C₁₇H₃₂O₄Na.

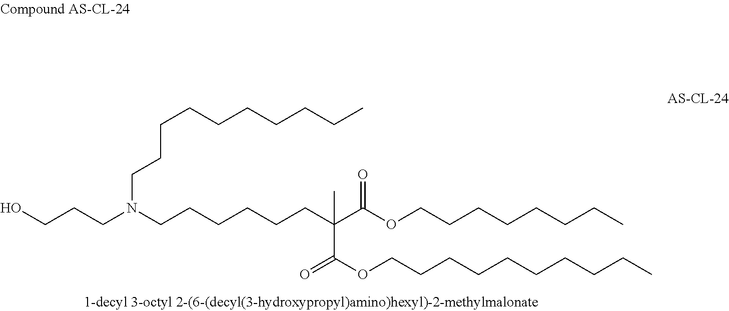 [SMILES: CCCCCCCCCCOC(=O)C(C)(CCCCCCN(CCCO)CCCCCCCCCC)C(=O)OCCCCCCCC]

Compound AS-CL-24 was synthesized according to the procedure in scheme 4 and the general step 8 procedure in scheme 2. The title compound was obtained as a colorless oil. ¹H NMR (600 MHz, CDCl₃) δ 4.08 (t, J=6.6 Hz, 4H), 3.78 (t, J=5.4 Hz, 2H), 2.62 (t, J=5.4 Hz, 2H), 2.38 (t, J=7.8 Hz, 4H), 1.84-1.81 (m, 2H), 1.67-1.63 (m, 2H), 1.62-1.57 (m, 4H), 1.47-1.41 (m, 4H), 1.38 (s, 3H), 1.29-1.18 (m, 44H), 0.88-0.86 (m, 9H). ¹³C NMR (150 MHz, CDCl₃) δ 172.6, 65.4, 64.9, 55.4, 54.3, 54.2, 53.8, 35.6, 32.0, 31.9, 30.0, 29.70, 29.69, 29.67, 29.63, 29.4, 29.30, 29.28, 29.26, 28.6, 27.8, 27.6, 27.4, 26.9, 26.8, 25.9, 24.4, 22.8, 22.7, 20.0, 14.2, 14.1. MS (ESI): m/z [M+H]⁺ 668.6192 for C₄₁CH₈₂NO₅.

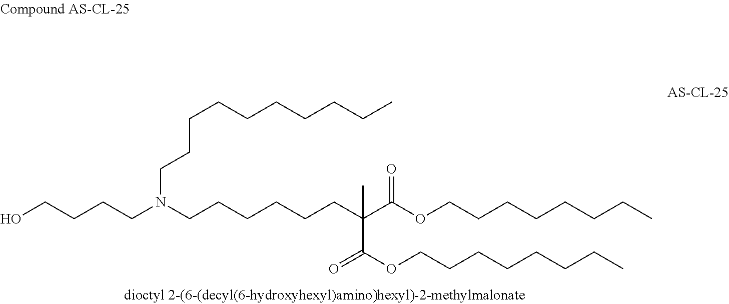 [SMILES: CCCCCCCCCCN(CCCCO)CCCCCCC(C)(C(=O)OCCCCCCCC)C(=O)OCCCCCCCC]

Compound AS-CL-25 was synthesized according to the general step 8 procedure in scheme 2. The title compound was obtained as a colorless oil. ¹H NMR (600 MHz, CDCl₃) δ 4.07 (t, J=6.6 Hz, 4H), 3.61 (t, J=6.6 Hz, 2H), 2.43-2.39 (m, 6H), 1.83-1.80 (m, 2H), 1.59-1.53 (m, 6H), 1.45-1.38 (m, 6H), 1.37 (s, 3H), 1.36-1.19 (m, 44H), 0.87-0.85 (m, 9H). ¹³C NMR (150 MHz, CDCl₃) δ 172.6, 65.4, 62.9, 54.12, 54.01, 53.95, 53.82, 35.6, 32.8, 32.0, 31.8, 29.9, 29.7, 29.6, 29.4, 29.3, 29.2, 28.6, 27.7, 27.4, 27.3, 26.64, 26.59, 26.55, 25.9, 25.7, 24.4, 22.73, 22.69, 19.9, 14.2, 14.1. MS (ESI): m/z [M+H]⁺ 682.6350 for C₄₂H₈₄NO₅.

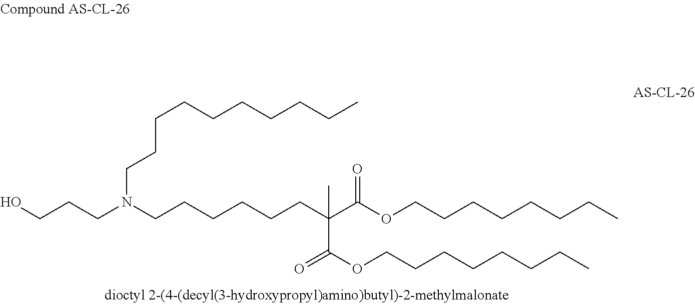 [SMILES: CCCCCCCCCCN(CCCO)CCCCCCC(C)(C(=O)OCCCCCCCC)C(=O)OCCCCCCCC]

Compound AS-CL-26 was synthesized according to the general step 8 procedure in scheme 2. The title compound was obtained as a colorless oil. ¹H NMR (600 MHz, CDCl₃) δ 4.08 (t, J=6.6 Hz, 4H), 3.76 (t, J=5.4 Hz, 2H), 2.60 (t, J=5.4 Hz, 2H), 2.39-2.36 (m, 4H), 1.86-1.83 (m, 2H), 1.65-1.63 (m, 2H), 1.60-1.56 (m, 4H), 1.48-1.41 (m, 4H), 1.37 (s, 3H), 1.31-1.18 (m, 36H), 0.87-0.85 (m, 9H). ¹³C NMR (150 MHz, CDCl3) δ 172.5, 65.4, 64.8, 55.3, 54.4, 54.0, 53.8, 35.6, 32.0, 31.9, 29.70, 29.67, 29.66, 29.4, 29.3, 29.2, 28.6, 27.9, 27.6, 27.2, 26.9, 25.9, 22.8, 22.7, 22.4, 20.0, 14.2, 14.1. MS (ESI): m/z [M+H]⁺ 612.5555 for C₃₇H₇₄NO₅.

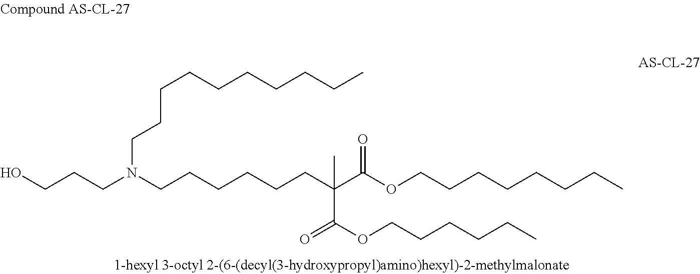 [SMILES: CCCCCCCCCCN(CCCO)CCCCCCC(C)(C(=O)OCCCCCC)C(=O)OCCCCCCCC]

Compound AS-CL-27 was synthesized according to the procedure in scheme 4 and the general step 8 procedure in scheme 2. The title compound was obtained as a colorless oil. ¹H NMR (600 MHz, CDCl₃) δ 4.08 (t, J=6.6 Hz, 4H), 3.78 (t, J=5.4 Hz, 2H), 2.61 (t, J=5.4 Hz, 2H), 2.38 (t, J=7.2 Hz, 4H), 1.84-1.81 (m, 2H), 1.66-1.64 (m, 2H), 1.60-1.58 (m, 4H), 1.45-1.42 (m, 4H), 1.38 (s, 3H), 1.32-1.18 (m, 36H), 0.89-0.86 (m, 9H). ¹³C NMR (150 MHz, CDCl₃) δ 172.6, 65.4, 64.9, 55.4, 54.3, 54.2, 53.8, 35.6, 32.0, 31.9, 31.5, 30.0, 29.69, 29.68, 29.66, 29.4, 29.3, 29.2, 28.6, 28.5, 27.8, 27.6, 27.4, 26.9, 26.8, 25.9, 25.6, 24.4, 22.8, 22.7, 22.6, 19.9, 14.2, 14.1, 14.0. MS (ESI): m/z [M+H]⁺ 612.5565 for C₃₇H₇₄NO₅.

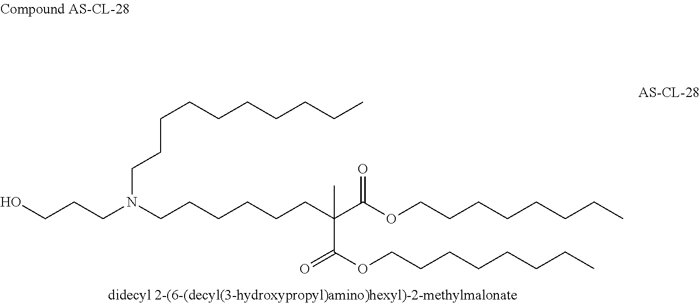 [SMILES: CCCCCCCCCCN(CCCO)CCCCCCC(C)(C(=O)OCCCCCCCC)C(=O)OCCCCCCCC]

Compound AS-CL-28 was synthesized according to the general step 8 procedure in scheme 2. The title compound was obtained as a colorless oil. ¹H NMR (600 MHz, CDCl₃) δ 4.07 (t, J=6.6 Hz, 4H), 3.77 (t, J=5.4 Hz, 2H), 2.61 (t, J=5.4 Hz, 2H), 2.37 (t, J=7.2 Hz, 4H), 1.83-1.81 (m, 2H), 1.67-1.63 (m, 2H), 1.61-1.56 (m, 4H), 1.46-1.41 (m, 4H), 1.37 (s, 3H), 1.32-1.18 (m, 48H), 0.87-0.85 (m, 9H). ¹³C NMR (150 MHz, CDCl₃) δ 172.6, 65.3, 64.9, 55.4, 54.3, 54.2, 53.8, 35.6 (×2), 32.0, 30.0, 29.69, 29.67, 29.65, 29.61 (×2), 29.4 (×2), 29.3, 28.6, 27.8, 27.6, 27.4, 26.9, 26.8, 25.9, 24.4, 22.7 (×2), 19.9, 14.2 (×2). MS (ESI): m/z [M+H]⁺ 696.6505 for C₄₃H₈₆NO₅.

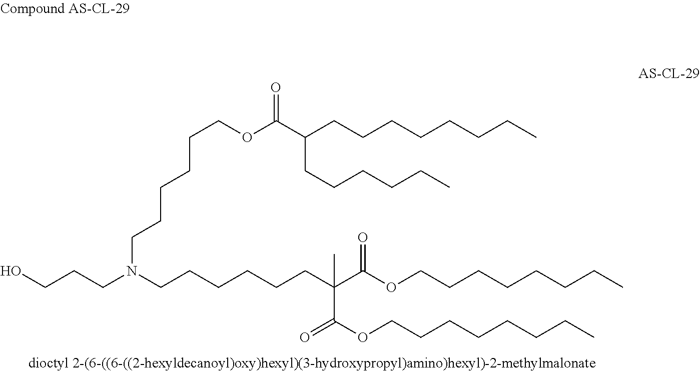 [SMILES: CCCCCCCCOC(=O)C(C)(CCCCCCN(CCCO)CCCCCCOC(=O)C(CCCCCC)CCCCCCCC)C(=O)OCCCCCCCC]

Compound AS-CL-29 was synthesized according to the general step 8 procedure in scheme 2. The title compound was obtained as a colorless oil. ¹H NMR (600 MHz, CDCl₃) δ 4.07 (t, J=6.6 Hz, 4H), 4.04 (t, J=6.6 Hz, 2H), 3.77 (t, J=5.4 Hz, 2H), 2.61 (t, J=5.4 Hz, 2H), 2.39-2.36 (m, 4H), 2.31-2.27 (m, 1H), 1.83-1.81 (m, 2H), 1.66-1.56 (m, 10H), 1.49-1.39 (m, 6H), 1.37 (s, 3H), 1.35-1.17 (m, 50H), 0.87-0.84 (m, 12H). ¹³C NMR (150 MHz, CDCl₃) δ 167.8, 172.6, 65.3, 64.9, 64.1, 55.3, 54.22, 54.19, 53.8, 45.9, 35.6, 32.6 (×2), 31.9, 31.8, 31.7, 30.0, 29.6, 29.5, 29.3, 29.28, 29.26, 29.24, 28.8, 28.6, 27.8, 27.5, 27.48, 27.43, 27.2, 26.9, 26.8, 26.0, 25.9, 24.4, 22.72, 22.69, 22.65, 19.9, 14.2, 14.1 (×2). MS (ESI): m/z [M+H]⁺ 838.7495 for C₅₁H₁₀₀NO₇.

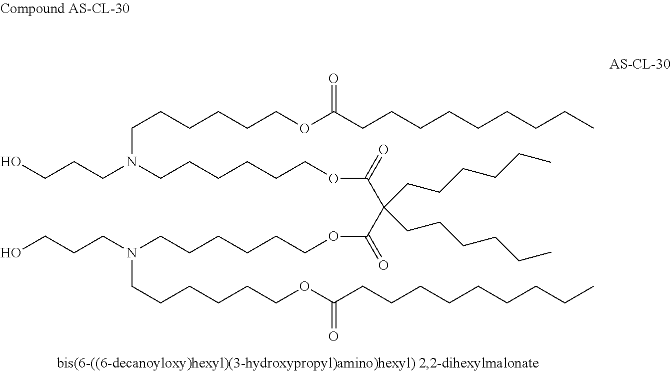 [SMILES: CCCCCCCCCC(=O)OCCCCCCN(CCCO)CCCCCCOC(=O)C(CCCCCC)(CCCCCC)C(=O)OCCCCCCN(CCCO)CCCCCCOC(=O)CCCCCCCCC]

Compound AS-CL-30 was synthesized according to the procedure in scheme 5.

A mixture solution of malonic acid (2 g, 19.21 mmol), 6-bromohexan-1-ol (7.65 g, 42.26 mmol), EDCI (8.1 g, 42.26 mmol) and DMAP (0.47 g, 3.84 mmol) in DCM was stirred at R.T overnight. After washing with 2N HCl_{(aq)} and evaporation, the residue was purified by silica gel column chromatography with EA/Hex (1/10) to yield the compound 10 (4.18 g, 50%) as a light-yellow oil. ¹H NMR (600 MHz, CDCl3) δ 4.14 (t, J=6.6 Hz, 4H), 3.40 (t, J=6.6 Hz, 4H), 3.36 (s, 2H), 1.88-1.84 (m, 4H), 1.69-1.64 (m, 4H), 1.50-1.44 (m, 4H), 1.41-1.35 (m, 4H). ¹³C NMR (150 MHz, CDCl₃) δ 166.6, 65.3, 41.5, 33.6, 32.5, 28.2, 27.6, 24.9.

A mixture solution of compound 10 (1.07 g, 2.49 mmol) and 1-iodohexane (2.11 g, 9.95 mmol) was stirred at R.T, and then the addition of NaH (0.2 g, 5 mmol). The reaction solution was stirred at R.T for 4 hours. After washing with sat. NH₄Cl_{(aq)} and evaporation, the residue was purified by silica gel column chromatography with EA/Hex (1/25) to yield the compound 11 (1 g, 67%) as a yellow oil. ¹H NMR (600 MHz, CDCl₃) δ 4.10 (t, J=6.6 Hz, 4H), 3.40 (t, J=6.6 Hz, 2H), 1.86-1.83 (m, 8H), 1.65-1.60 (m, 4H), 1.48-1.43 (m, 4H), 1.38-1.33 (m, 4H), 1.33-1.22 (m, 12H), 1.17-1.09 (m, 4H), 0.88-0.86 (m, 6H). ¹³C NMR (150 MHz, CDCl₃) δ 172.0, 64.8, 57.7, 33.5, 32.6, 32.2, 31.5, 29.5, 28.3, 27.7, 25.1, 23.9, 22.5, 14.0. MS (ESI): m/z [M+Na]⁺ 619.1967, [M+Na]²⁺ 621.1942 and [M+Na]⁴⁺ 623.1930 for C₂₇H₅₀O₄Br₂Na.

A mixture solution of compound 11 (160 mg, 0.267 mmol), KI (90 mg, 0.534 mmol) and 3-amino-1-propanol (1 g, 13.37 mmol) in MeCN/DCM was stirred at R.T for 4 hours. After evaporation, the residue was dissolved in DCM and washing with water and brine. After removing the solvent, the crude was mixed with 6-bromohexyl decanoate (212 mg, 0.633 mmol), KI (105 mg, 0.633 mmol) and K₂CO₃ (320 mg, 1.15 mmol) in MeCN/DCM. The mixture solution was heated at 50° C. overnight. After evaporation, the residue was purified by silica gel column chromatography with 5% MeOH/DCM/1% NH₄OH to yield the title compound AS-Cl-30 (180 mg, 57%) as a colorless oil. ¹H NMR (600 MHz, CDCl₃) δ 4.06 (t, J=6.6 Hz, 4H), 4.03 (t, J=6.6 Hz, 4H), 3.76 (br. s, 4H), 2.60 (t, J=5.4 Hz, 4H), 2.37 (t, J=7.8 Hz, 8H), 2.26 (t, J=7.8 Hz, 4H), 1.84-1.81 (m, 4H), 1.65-1.56 (m, 16H), 1.48-1.42 (m, 8H), 1.37-1.24 (m, 52H), 1.14-1.07 (m, 4H), 0.86-0.84 (m, 6H). ¹³C NMR (150 MHz, CDCl₃) δ 174.0, 172.1, 65.0, 64.8, 64.3, 57.7, 55.3, 54.2 (×2), 34.4, 32.3, 31.9, 31.6, 29.6, 29.5, 29.3 (×2), 29.2 (×2), 28.7, 28.6, 27.9, 27.2 (×2), 26.8, 26.0, 25.9, 25.0, 24.0, 22.7, 22.6, 14.14, 14.09. MS (ESI): m/z [M+H]⁺ 1095.9487 for C₆₅H₁₂₇N₂O₁₀.

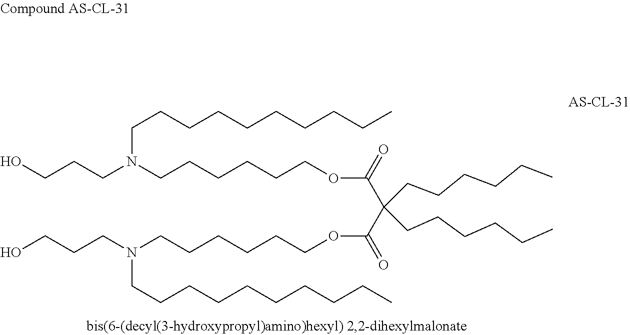 [SMILES: CCCCCCCCCCN(CCCO)CCCCCCOC(=O)C(CCCCCC)(CCCCCC)C(=O)OCCCCCCN(CCCO)CCCCCCCCCC]

Compound AS-CL-31 was synthesized according to the procedure of AS-CL-30. The title compound was obtained as a colorless oil. ¹H NMR (600 MHz, CDCl₃) δ 4.09-4.06 (m, 4H), 3.78-3.76 (m, 4H), 2.62-2.38 (m, 4H), 2.38 (br. s, 8H), 1.85-1.83 (m, 4H), 1.66-1.65 (m, 4H), 1.63-1.58 (m, 4H), 1.49-1.41 (m, 8H), 1.36-1.22 (m, 48H), 1.14-1.11 (m, 4H), 0.86-0.85 (m, 12H). ¹³C NMR (150 MHz, CDCl₃) δ 172.2, 65.1, 64.9, 57.8, 55.3, 54.3, 54.2, 32.3, 32.0, 31.6, 29.7 (×2), 29.66, 29.6, 29.4, 28.6, 27.9, 27.6, 27.2, 26.9, 26.8, 26.0, 24.0, 22.8, 22.6, 14.2, 14.1. MS (ESI): m/z [M+H]⁺ 868.0482 for C₅₃H₁₀₇N₂O₆.

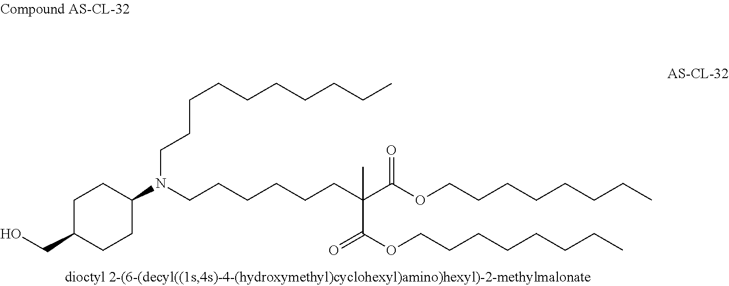 [SMILES: CCCCCCCCCCN(CCCCCCC(C)(C(=O)OCCCCCCCC)C(=O)OCCCCCCCC)[C@H]1CC[C@@H](CO)CC1]

Compound AS-CL-32 was synthesized according to the general step 8 procedure in scheme 2. The title compound was obtained as a colorless oil. ¹H NMR (600 MHz, CDCl₃) δ 4.07 (t, J=6.6 Hz, 4H), 3.59 (d, J=7.2 Hz, 2H), 2.53-2.45 (m, 5H), 1.83-1.80 (m, 2H), 1.78-1.71 (m, 2H), 1.61-1.55 (m, 6H), 1.45-1.38 (m, 6H), 1.37 (s, 3H), 1.32-1.18 (m, 42H), 0.87-0.85 (m, 9H). ¹³C NMR (150 MHz, CDCl₃) δ 172.6, 65.3, 64.4, 59.4, 53.8, 50.6, 50.4, 36.1, 35.6, 31.9, 31.8, 29.9, 29.7, 29.6, 29.4, 29.24, 29.22, 28.6, 27.6, 27.4, 25.9, 24.6, 24.4, 22.72, 22.67, 19.9, 14.2, 14.1. MS (ESI): m/Z [M+H]⁺ 694.6339 for C₄₃H₈₄NO₅.

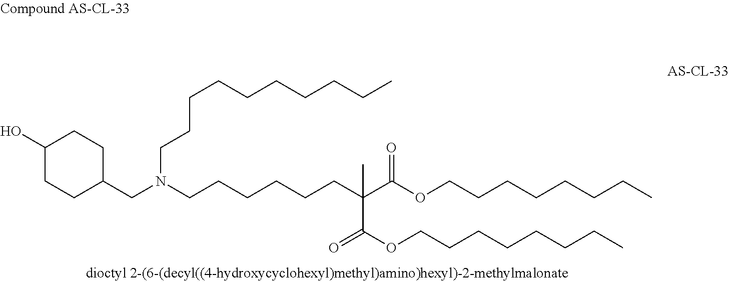 [SMILES: CCCCCCCCCCN(CCCCCCC(C)(C(=O)OCCCCCCCC)C(=O)OCCCCCCCC)CC1CCC(O)CC1]

Compound AS-CL-33 was synthesized according to the general step 8 procedure in scheme 2. The title compound was obtained as a colorless oil. ¹H NMR (600 MHz, CDCl₃) δ 4.07-4.01 (m, 4H), 3.88 (br. s, 0.5H), 3.48-3.46 (m, 0.5H), 2.27-2.25 (m, 4H), 2.13-2.12 (m, 1H), 2.06-2.05 (m, 1H), 1.92-1.90 (m, 1H), 1.80-1.77 (m, 3H), 1.63-1.48 (m, 7H), 1.41-1.14 (m, 48H), 0.84-1.81 (m, 9H). ¹³C NMR (150 MHz, CDCl₃) δ 172.5, 71.2, 67.3, 65.2, 61.0, 60.2, 54.9, 54.8, 54.7, 53.7, 35.6, 35.5, 35.4, 34.7, 32.2, 31.9, 31.8, 29.9, 29.7, 29.6, 29.3, 29.2, 29.1, 28.5, 27.52, 27.49, 27.3, 27.2, 27.1, 27.0, 25.8, 25.6, 24.3, 22.7, 22.6, 19.8, 14.1, 14.0. MS (ESI): m/z [M+H]⁺ 694.6353 for C₄₃H₈₄NO₅.

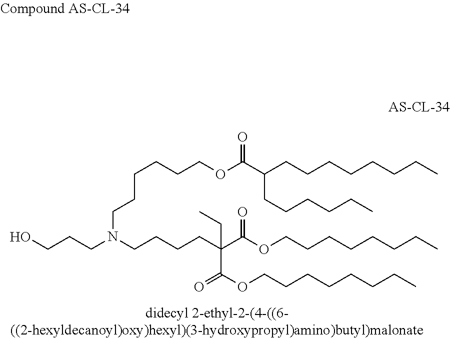 [SMILES: CCCCCCCCOC(=O)C(CC)(CCCCN(CCCO)CCCCCCOC(=O)C(CCCCCC)CCCCCCCC)C(=O)OCCCCCCCC]

Compound AS-CL-34 was synthesized according to the general step 8 procedure in scheme 2. The title compound was obtained as a colorless oil. ¹H NMR (600 MHz, CDCl₃) δ 4.09-4.02 (m, 6H), 3.76-3.75 (m, 2H), 2.61 (br. s, 2H), 2.39 (br. s, 4H), 2.29-2.27 (m, 1H), 1.90 (q, J=7.8 Hz, 2H), 1.87-1.84 (m, 2H), 1.65-1.55 (m, 10H), 1.47-1.12 (m, 60H), 0.87-0.85 (m, 12H), 0.79 (t, J=7.8 Hz, 3H). ¹³C NMR (150 MHz, CDCl₃) δ 176.7, 171.9, 65.2, 64.6, 64.0, 58.0, 55.1, 54.3, 54.0, 45.9, 32.6, 31.93, 31.90, 31.7, 29.6 (×2), 29.5, 29.4, 29.3, 29.27, 28.7, 28.6, 27.9, 27.5, 27.4, 27.2, 26.8, 26.0, 25.9, 25.3, 22.7, 22.6, 22.1, 14.1. MS (ESI): m/z [M+H]⁺ 880.7963 for C₅₄H₁₀₆NO₇.

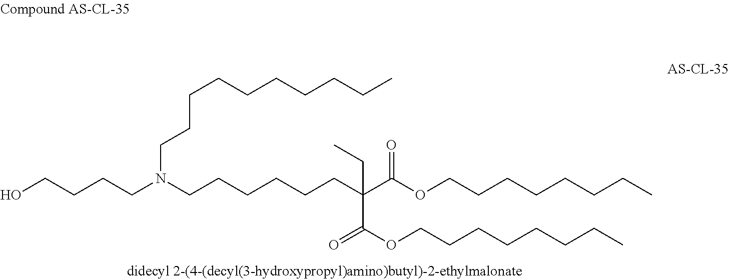 [SMILES: CCCCCCCCCCN(CCCCO)CCCCCCC(CC)(C(=O)OCCCCCCCC)C(=O)OCCCCCCCC]

Compound AS-CL-35 was synthesized according to the general step 8 procedure in scheme 2. The title compound was obtained as a colorless oil. 4.09-4.06 (m, 4H), 3.76 (t, J=4.8 Hz, 2H), 2.64 (br. s, 2H), 2.43-2.40 (m, 4H), 1.90 (q, J=7.8 Hz, 2H), 1.87-1.84 (m, 2H), 1.68-1.66 (m, 2H), 1.60-1.56 (m, 4H), 1.50-1.45 (m, 4H), 1.28-1.24 (m, 42H), 1.16-1.10 (m, 2H), 0.87-0.85 (m, 9H), 0.79 (J=7.8 Hz, 3H). ¹H NMR (600 MHz, CDCl₃) δ 171.9, 65.3, 64.4, 58.1, 55.0, 54.2, 53.9, 32.0, 31.7, 29.7, 29.6 (×2), 29.4, 29.3, 28.6, 27.8, 27.5, 27.0, 26.7, 25.9, 25.4, 22.7, 22.1, 14.1, 8.5. MS (ESI): m/z [M+H]⁺ 682.6341 for C₄₂H₈₄NO₅.

### Example 2 Preparation and Characterization of Nucleic Acid Loaded Lipid Nanoparticles

2.1 Preparation of mRNA-Lipid Nanoparticle (mRNA-LNP) Complexes

LNPs comprised of commercially available ionizable lipids were formulated with a total lipid concentration of 50 mM MC3-, SM-102, AS-CL05, AS-CL09, AS-CL28 or AS-CL35. LNPs were formulated with (DLin-MC3-DMA, SM-102, AS-CL05, AS-CL09 AS-CL28 or AS-CL35)/DSPC/Cholesterol/DMG-PEG2000 in molar ratio of 50/10/38.5/1.5. Each lipid was dissolved in ethanol and mixed according to the specified molar ratios in the organic phase.

The LNPs were assembled with mRNA using NanoAssmblr™ (Precision NanoSystems) Ignite microfluidic mixing device. Two different mRNA targets were encapsulated in LNPs: (i) the mRNA of SARS-CoV-2 S protein from WT and Omicron BA.5; and (ii) the mRNA encoding DENV2 serotype envelope (E) protein (DENV2 E mRNA). To this purpose, the mRNA was dissolved in 50 mM sodium acetate buffer (pH 4.5), with the NP lipid:mRNA ratio at a constant value of 6.5, prior to mixing in the Spark NanoAssmblr™ (Precision NanoSystems). A 16-μL aliquot of the organic phase and a 32-μL aliquot of the aqueous phase were mixed and ejected into 48 μL of PBS at pH 7.4. The LNPs were then diluted into an additional 96 μL DPBS at pH 7.4 and dialyzed against PBS.

2.2 Physiochemical Characterization of mRNA-LNP Complexes

The mRNA-LNP complexes of Example 2.1 were diluted 100-fold in PBS (pH 7.4) and transferred into a 384-well microplate for size and polydispersity index (PDI) measurements by Dynamic Light Scattering (DLS). Encapsulation efficiency was evaluated by disrupting each complex with 1% Triton X-100 to release the mRNA cargo therein. Results are summarized in Table 2.

TABLE 2
Physiochemical characterization of the
mRNA-LNP complexes of Example 2.1

| | Parameters | | |
|---|---|---|---|
| | | | Encapsulation |
| Formulation | Size (nm) | PDI | efficiency (%) |
| MC3-LNP | 73 | 0.19 | 98 |
| SM-102-LNP | 72 | 0.18 | 96 |
| AS-CL05-LNP | 94 | 0.22 | 97 |
| AS-CL09-LNP | 81 | 0.13 | 90 |
| AS-CL28-LNP | 96 | 0.21 | 95 |
| AS-CL35-LNP | 104 | 0.15 | 90 |

The data in Table 2 confirmed that the present cationic lipid, AS-CL05, AS-CL09, AS-CL28 and AS-CL35 could respectively associate with other helper lipids to form nanoparticles. The physical characteristics of AS-CL05-LNPs, AS-CL09-LNPs, AS-CL28-LNPs and AS-CL35-LNPs including size and polydispersity index (PDI), were similar to those of other LNPs composed of commercially available cationic lipids. The average particle sizes of mRNA-LNP complexes were found to be between 70-90 nm, PDI values for all complexes were lower than 0.3, indicating a uniform distribution and lack of aggregation in aqueous solution.

### 2.3 In Vitro and In Vivo Protein Expression

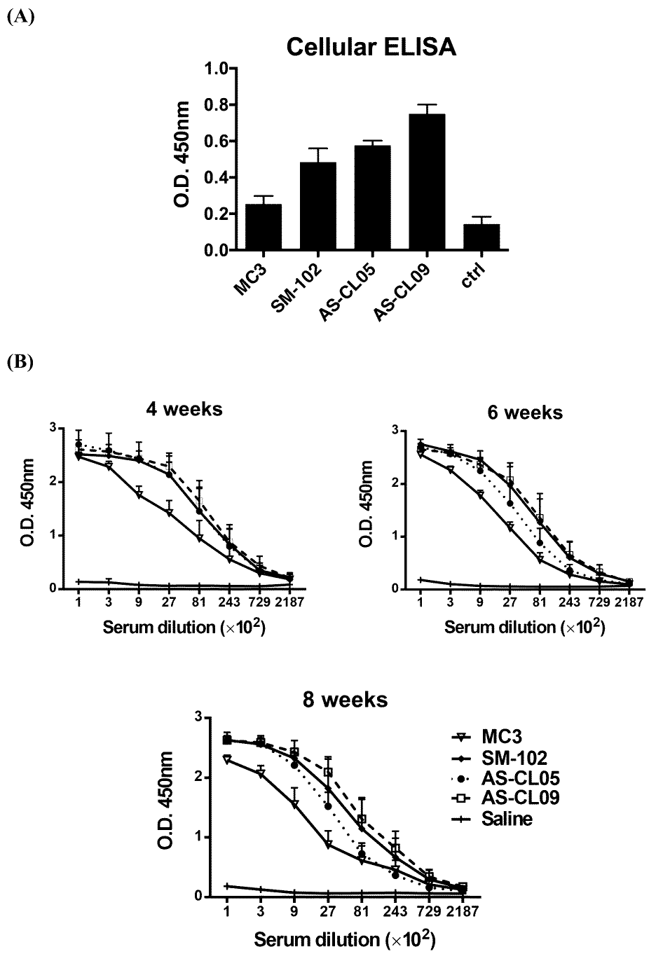

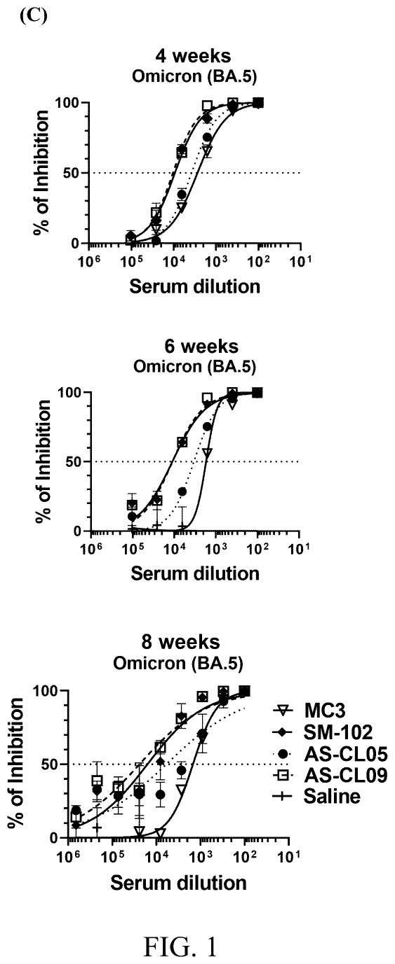

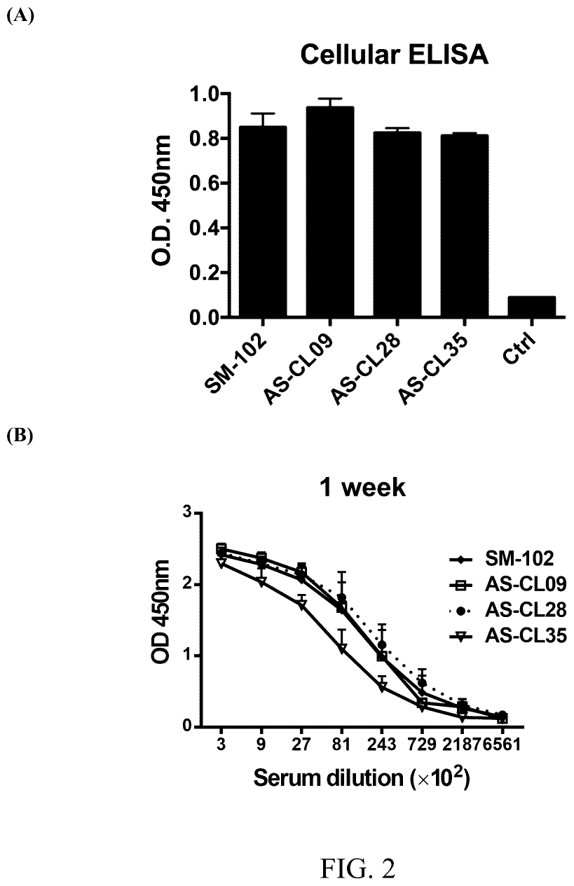

To assess mRNA transfection and protein expression in vitro, 293T cells were treated with mRNA-LNP complexes of Example 2.1, and cell lysates were collected and analyzed by flow cytometry. It was found that each mRNA-LNP complex could induce S protein expression in the transfected cells. Among the tested lipids, LNPs formed by the present cationic lipid—AS-CL09 exhibited the highest protein expression level, followed by MC3, SM102, AS-CL05, AS-CL28 and AS-CL35 (FIGS. 1A and 2A).

After confirming that each of the mRNA-LNP complexes could successfully induce protein expression in the cell-based assay, the immunogenic effects were assessed in BALB/c mice. Mice receiving saline served as negative controls. An ELISA-based method was performed to evaluate the binding of serum antibodies to WT recombinant S proteins and BA.5 recombinant S proteins. The antibodies elicited by the WT mRNA-AS-CL09 LNPs and the BA.5 mRNA-AS-CL09 LNPs exhibited the highest binding efficiency, while complexes respectively formed by MC3, SM-102, AS-CL05, AS-CL28 and AS-CL35 induced antibodies with slightly lower binding efficiencies (FIGS. 1B and 2B).

### 2.4 Serum Neutralizing Activity Against SARS-CoV-2 Variant Pseudovirus

Neutralizing activity was evaluated using sera collected from post-vaccinated animals. In the pseudovirus neutralization experiments, sera from mice immunized with BA.5 mRNA-AS-CL09 LNP complex exhibited the highest neutralizing ability. In comparison with MC3 and AS-CL05 groups, the sera from mice immunized with BA.5 mRNA-AS-CL09 LNP complexes had 9-fold higher neutralizing antibody titers against the BA.5 pseudovirus. Additionally, AS-CL09-containing LNPs had a better neutralizing antibody profile than SM-102 LNPs (FIG. 1C and Table 3). Thus, the type of ionizable lipid in the LNP greatly affected the final outcome of serum neutralizing activity, with AS-CL09 exhibiting the best performance among tested lipids.

TABLE 3
Half maximum inhibitory concentration (IC₅₀) of mRNA-LNP
complexes of Example 2.1 evaluated by SARS-CoV-2 pseudovirus
SARS-CoV-2 pseudovirus IC₅₀ (Serum dilution)

| | MC3-LNP | SM102-LNP | AS-CL05-LNP | AS-CL09LNP |
|---|---|---|---|---|
| 4 weeks | 4566 ± 109 | 9379 ± 202 | 5587 ± 303 | 11138 ± 769 |
| 6 weeks | 1651 ± 60 | 11566 ± 531 | 5636 ± 235 | 12583 ± 989 |
| 8 weeks | 1675 ± 165 | 13695 ± 659 | 7424 ± 826 | 15646 ± 886 |

2.5 Comparison of DENV2 E mRNA-AS-CL09 LNP Complexes and DENV2 E mRNA-SM-102 LNP Complexes

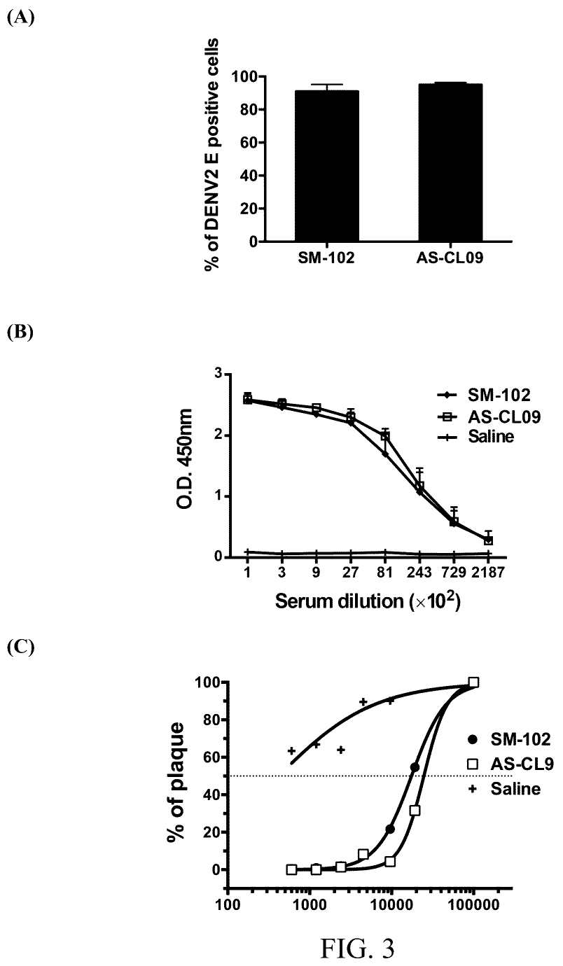

In this example, 293T cells were treated with DENV2 E protein mRNA-LNP complexes derived from AS-CL09 or SM-102 lipids, and analyzed for protein expression by flow cytometry using in-house monoclonal antibody against the DENV2 E protein (DB32-6). Results showed that DENV2 E protein was successfully expressed after transfection with either mRNA-LNP complex (FIG. 3A), and the protein expression levels were similar for both mRNA-LNP complexes.

Next, BALB/c mice were immunized with the mRNA-LNP complexes by intramuscular injection in accordance with procedures described in the “Materials and methods” section, and serum samples were collected on week 6 to evaluate the neutralizing antibody binding activity by ELISA. It was found that sera of mice inoculated with DENV2 E mRNA-AS-CL09 LNP exhibited higher binding activity against DENV2 serotype virus when compared with sera from DENV2 E mRNA-SM-102 LNP-vaccinated mice (FIG. 3B). The plaque reduction neutralization titer (PRNT) assay was used to evaluate the neutralizing activity of DENV antibodies against BHK-21 cells. Neutralizing antibodies were found at a high level in mice injected with DENV2 E mRNA-AS-CL09 LNPs. For the sample, PRNT50 values were around 24,725, which was approximately 1.4-times higher than the value from DENV2 E mRNA-SM-102 LNP sera (FIG. 3C and Table 4).

TABLE 4
Half maximum inhibitory concentration (IC₅₀) of DENV2
E mRNA-AS-CL09 LNP complexes evaluated by PRNT assay

| | PRNT₅₀ |
|---|---|
| | (Serum dilution) |
| Saline | <150 |
| SM-102 | 17,448 |
| AS-CL09 | 24,725 |

Based on these results, we concluded that compared with SM-102-containing mRNA-LNP complexes, mRNA-LNPs containing AS-CL09 ionizable lipid exhibited higher antibody production after intramuscular vaccination.

Taken together, the AS-CL05-LNPs, AS-CL09-LNPs, AS-CL28-LNPs and AS-CL35-LNPs could efficiently deliver mRNA encoding the SARS-CoV-2 spike protein or DENV E protein for expression. Furthermore, in terms of mRNA delivery efficiency, AS-CL05-LNPs, AS-CL09-LNPs, AS-CL28-LNPs and AS-CL35-LNPs may be comparable with other LNPs composed of lipids from commercial sources (i.e., MC3-, SM-102 or ALC-0315). Accordingly, the present cationic lipids (e.g., AS-CL05-LNPs, AS-CL09-LNPs, AS-CL28-LNPs and AS-CL35-LNPs) are useful for the manufacture of LNPs for gene and drug delivery.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

1. A lipid having the structure of formula (I), 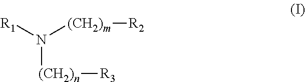
wherein,
R₁ is alkyl or cycloalkyl optionally substituted with one or more hydroxyl, —CH₂OH, 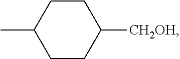
or —NR₂ groups;
m and n are independently an integral between 0 and 12;
R₂ and R₃ are independently H, alkeneyl, R^{a}, —(C═O)OCH₂R^{a}, —O(C═O)R^{b}, or —(C═O)OR^{b};
R^{a} is —CR′(COOR″)₂ or —CR′(COOR″)(COOR′″); and
R, R^{b}, R′, R″, and R′″ are independently H or alkyl.

2. The lipid of claim 1, wherein the lipid is selected from the group consisting of,

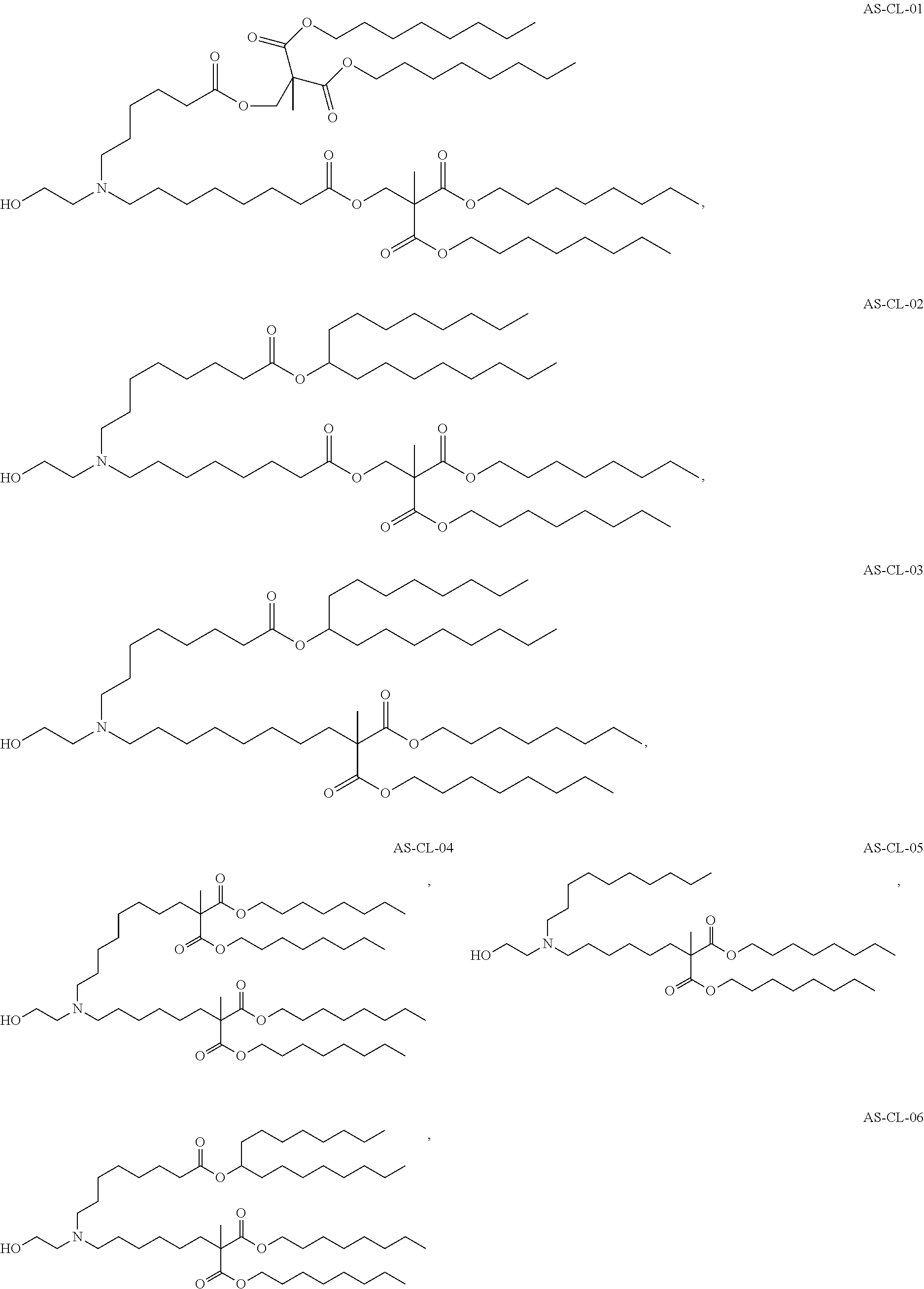

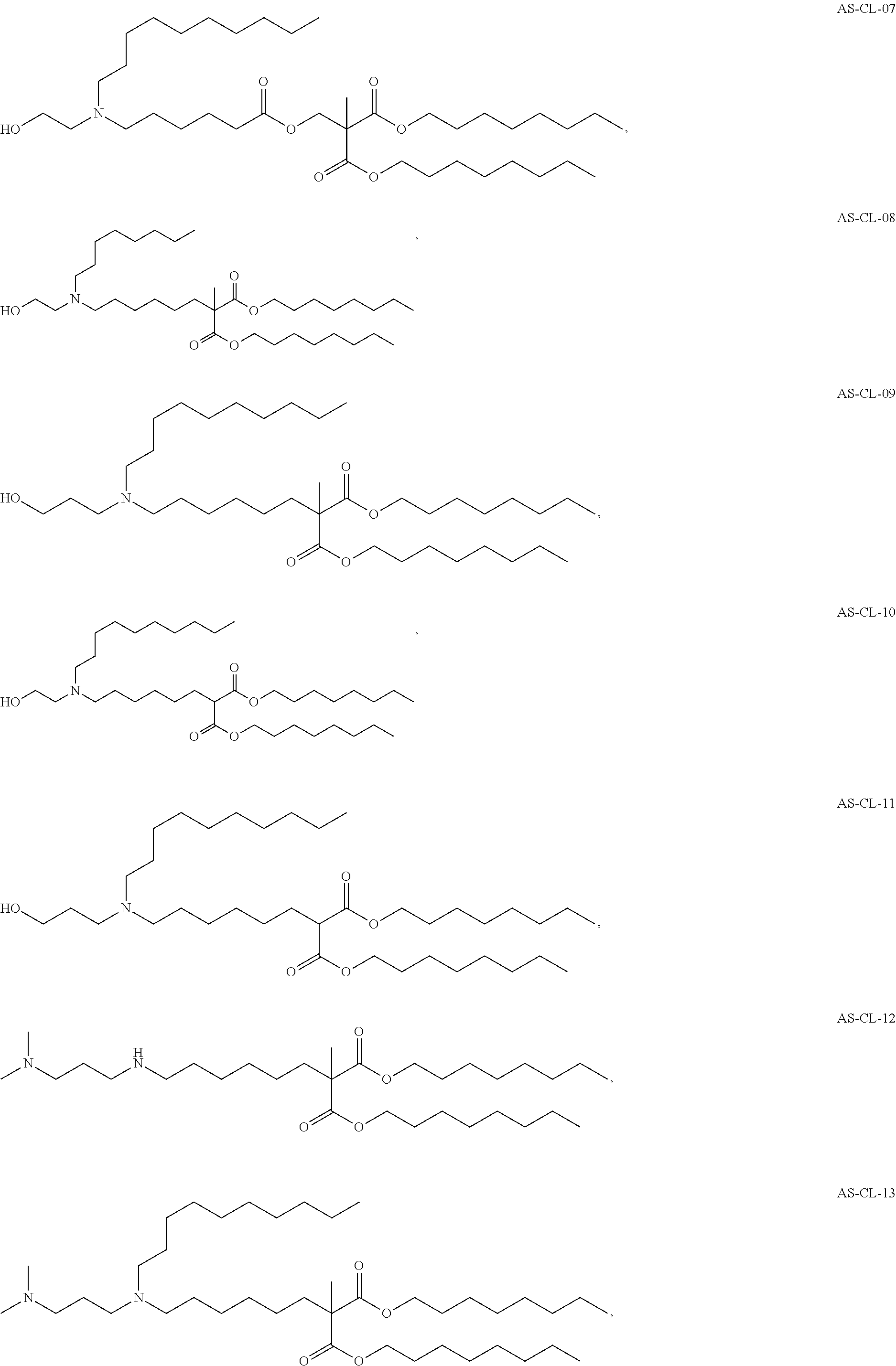

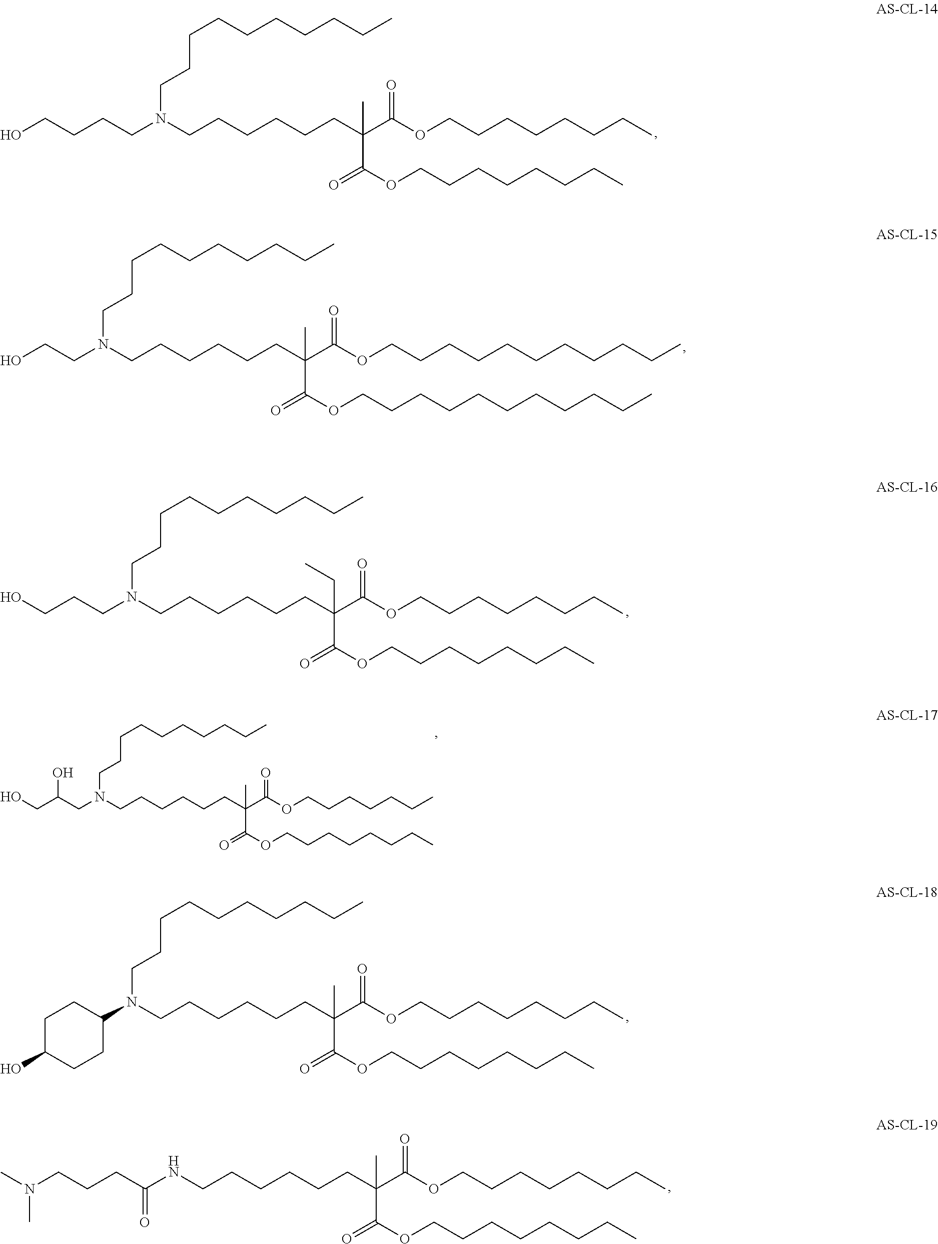

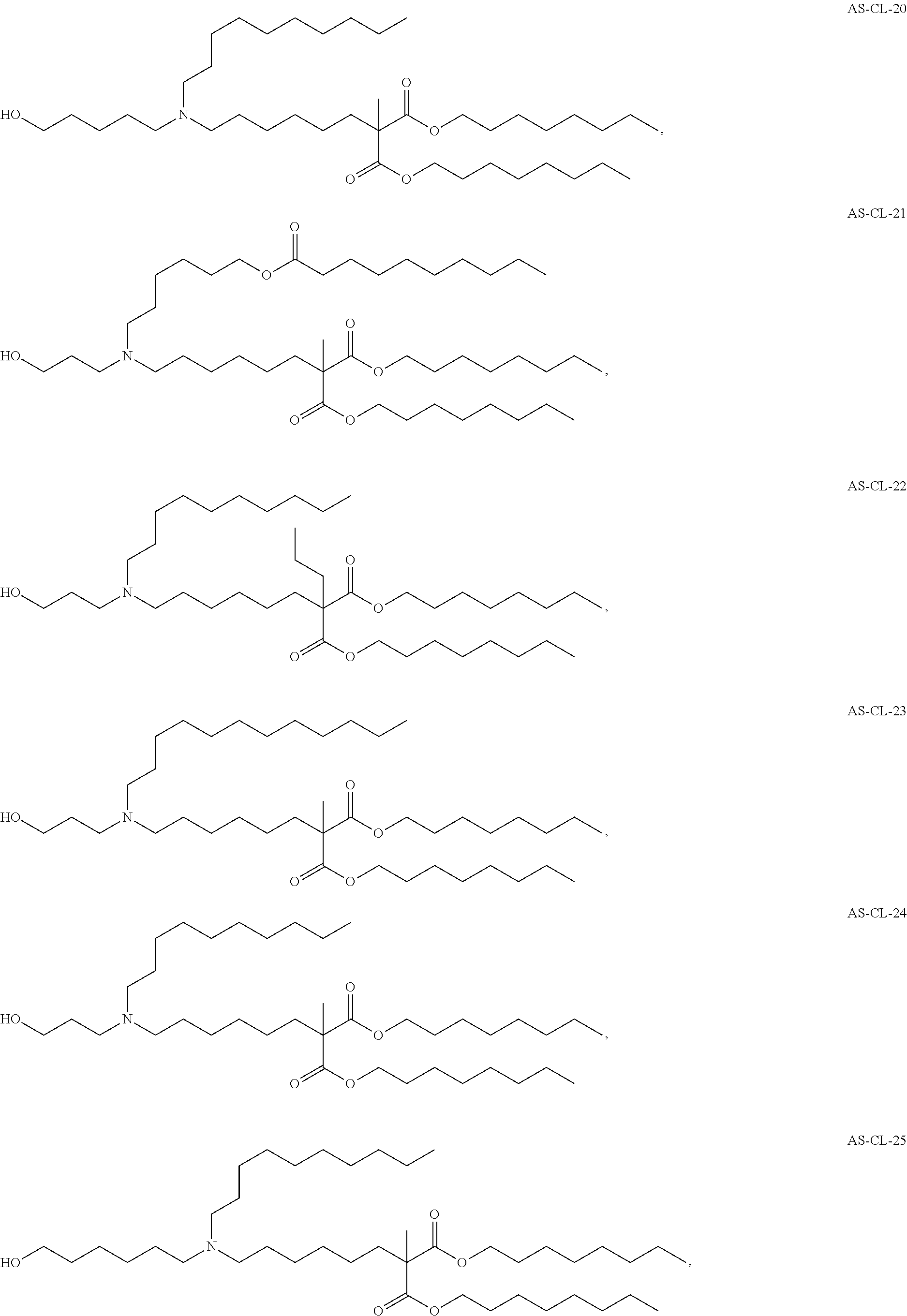

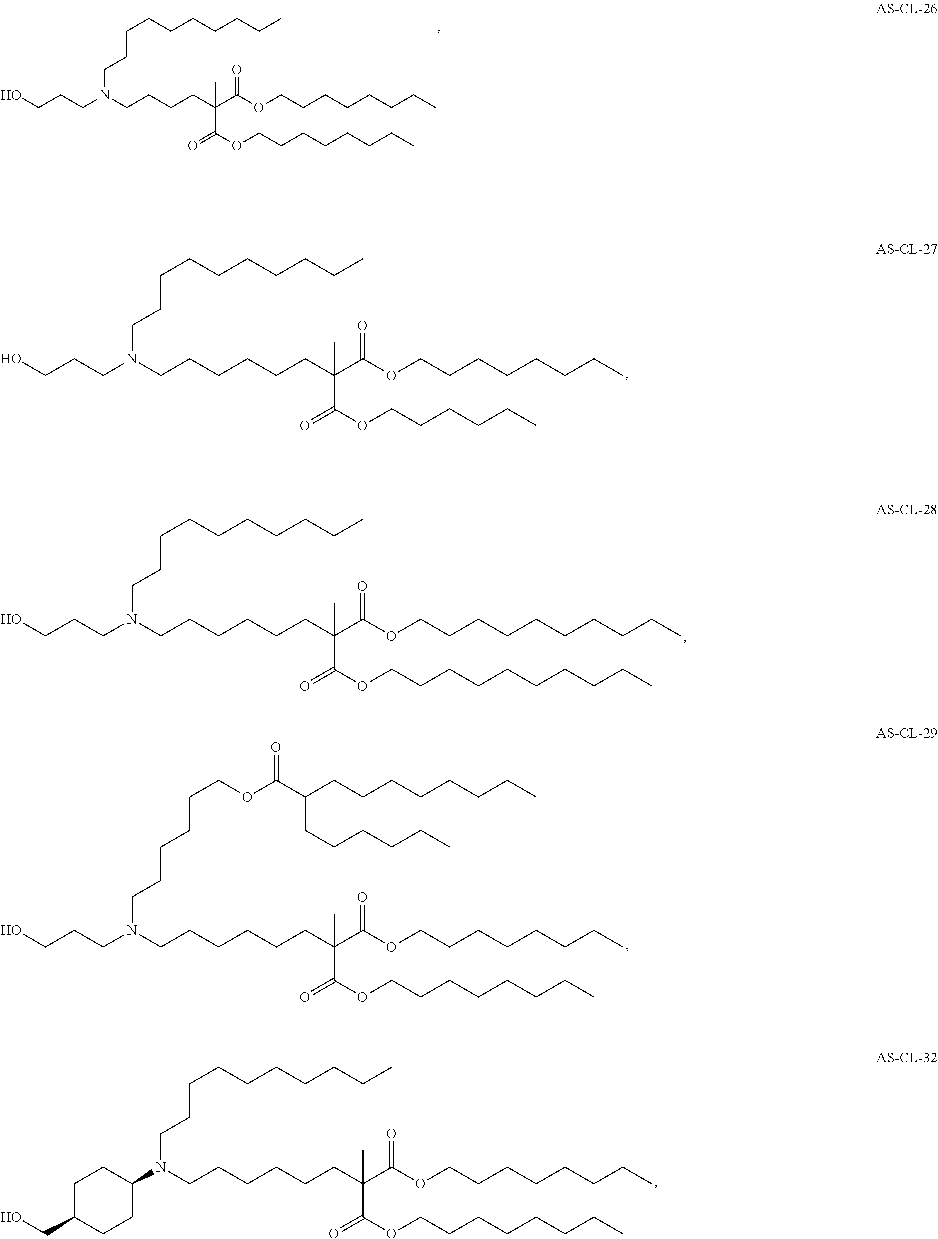 [SMILES: CCCCCCCCCCN(CCCO)CCCCC(C)(C(=O)OCCCCCCCC)C(=O)OCCCCCCCC ; CCCCCCCCCCN(CCCO)CCCCCCC(C)(C(=O)OCCCCCC)C(=O)OCCCCCCCC ; CCCCCCCCCCOC(=O)C(C)(CCCCCCN(CCCO)CCCCCCCCCC)C(=O)OCCCCCCCCCC ; CCCCCCCCOC(=O)C(C)(CCCCCCN(CCCO)CCCCCCOC(=O)C(CCCCCC)CCCCCCCC)C(=O)OCCCCCCCC ; CCCCCCCCCCN(CCCCCCC(C)(C(=O)OCCCCCCCC)C(=O)OCCCCCCCC)[C@H]1CC[C@@H](CO)CC1]

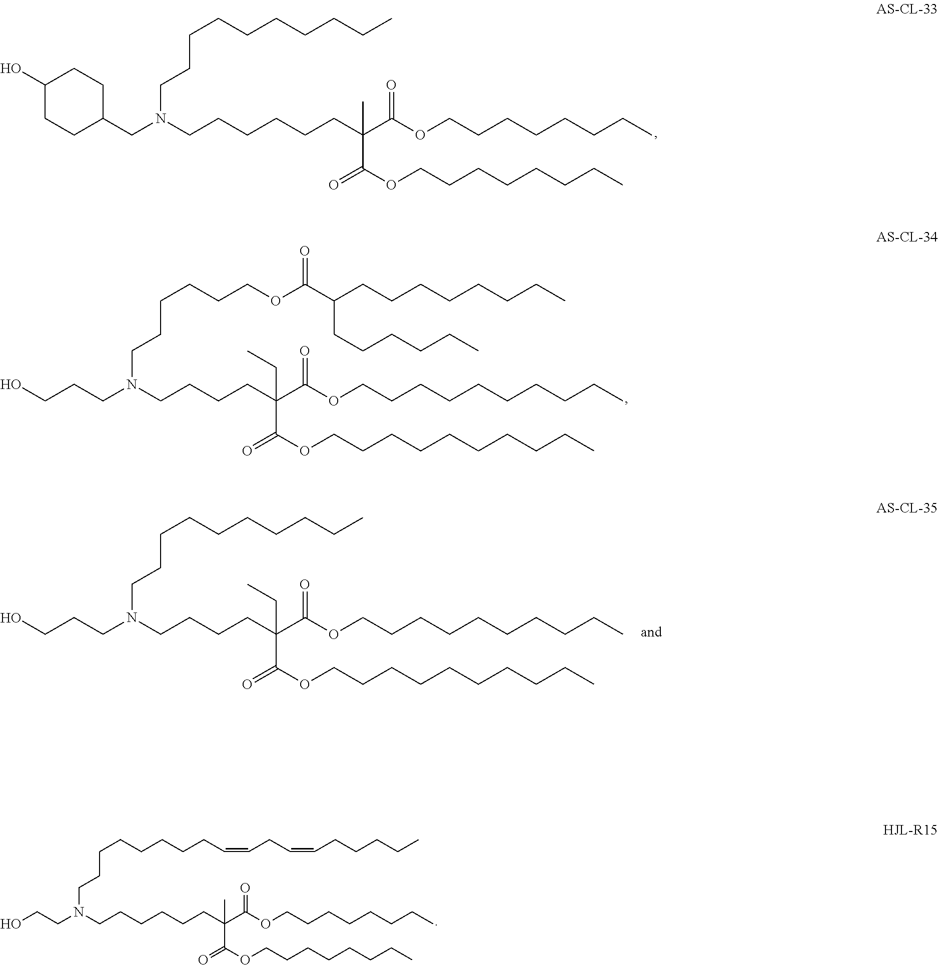 [SMILES: CCCCCCCCCCN(CCCCCCC(C)(C(=O)OCCCCCCCC)C(=O)OCCCCCCCC)CC1CCC(O)CC1 ; CCCCCCCCCCOC(=O)C(CC)(CCCCN(CCCO)CCCCCCOC(=O)C(CCCCCC)CCCCCCCC)C(=O)OCCCCCCCCCC ; CCCCCCCCCCOC(=O)C(CC)(CCCCN(CCCO)CCCCCCCCCC)C(=O)OCCCCCCCCCC ; CCCCC/C=C\C/C=C\CCCCCCCCN(CCO)CCCCCCC(C)(C(=O)OCCCCCCCC)C(=O)OCCCCCCCC]

3. (canceled)

4. (canceled)

5. (canceled)

6. (canceled)

7. A lipid nanoparticle having a hydrophilic core, and an outer lipid bilayer shell formed by one or more of the lipids of claim 1.

8. The lipid nanoparticle of claim 7, further comprising a therapeutic agent disposed in the hydrophilic core or the outer lipid bilayer shell of the lipid nanoparticle.

9. The lipid nanoparticle of claim 8, wherein the therapeutic agent is a nucleic acid of a viral protein.

10. The lipid nanoparticle of claim 9, wherein the nucleic acid is mRNA of spike protein of SARS-CoV-2.

11. The lipid nanoparticle of claim 10, wherein the nucleic acid is mRNA of an envelope protein of Dengue virus.

12. The lipid nanoparticle of claim 7, wherein the lipid is selected from the group consisting of, 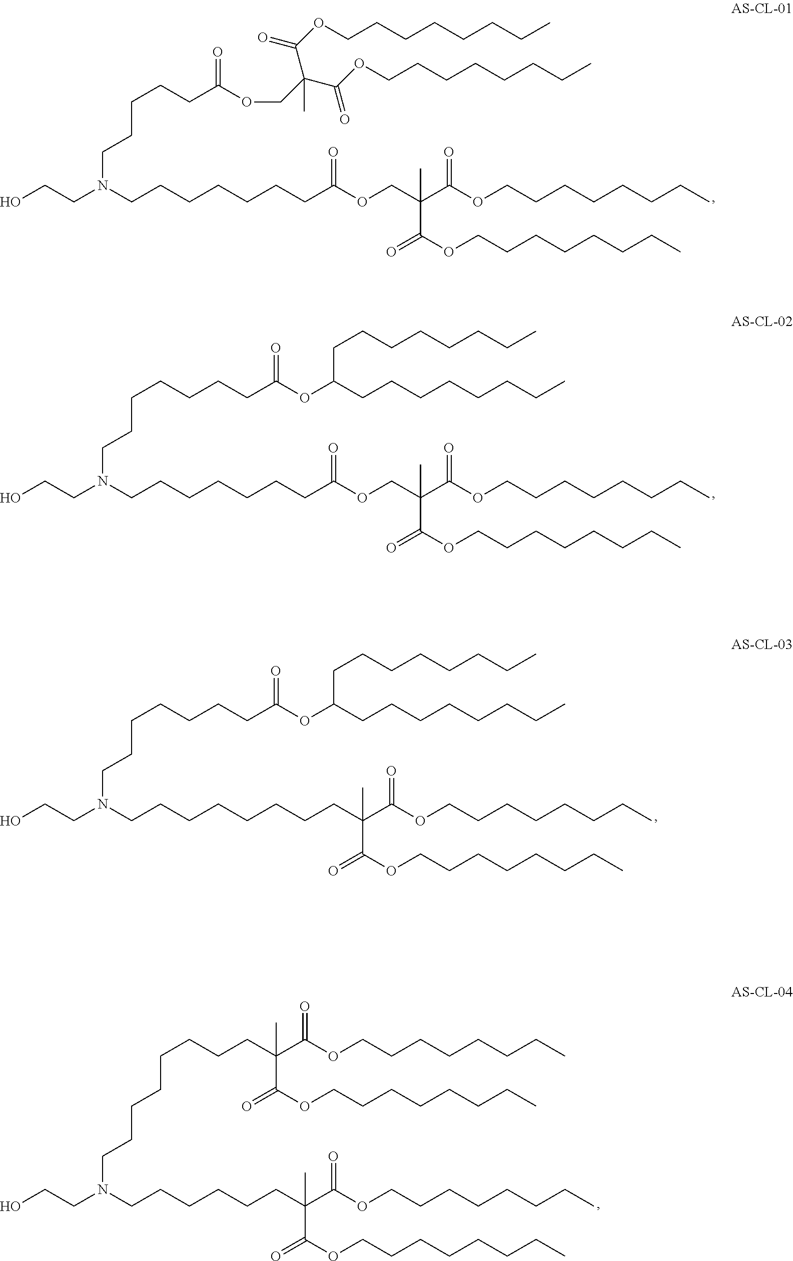 [SMILES: CCCCCCCCOC(=O)C(C)(COC(=O)CCCCCCCN(CCO)CCCCCC(=O)OCC(C)(C(=O)OCCCCCCCC)C(=O)OCCCCCCCC)C(=O)OCCCCCCCC ; CCCCCCCCOC(=O)C(C)(COC(=O)CCCCCCCN(CCO)CCCCCCCC(=O)OC(CCCCCCCC)CCCCCCCC)C(=O)OCCCCCCCC ; CCCCCCCCOC(=O)C(C)(CCCCCCCCN(CCO)CCCCCCCC(=O)OC(CCCCCCCC)CCCCCCCC)C(=O)OCCCCCCCC ; CCCCCCCCOC(=O)C(C)(CCCCCCCCN(CCO)CCCCCCC(C)(C(=O)OCCCCCCCC)C(=O)OCCCCCCCC)C(=O)OCCCCCCCC] 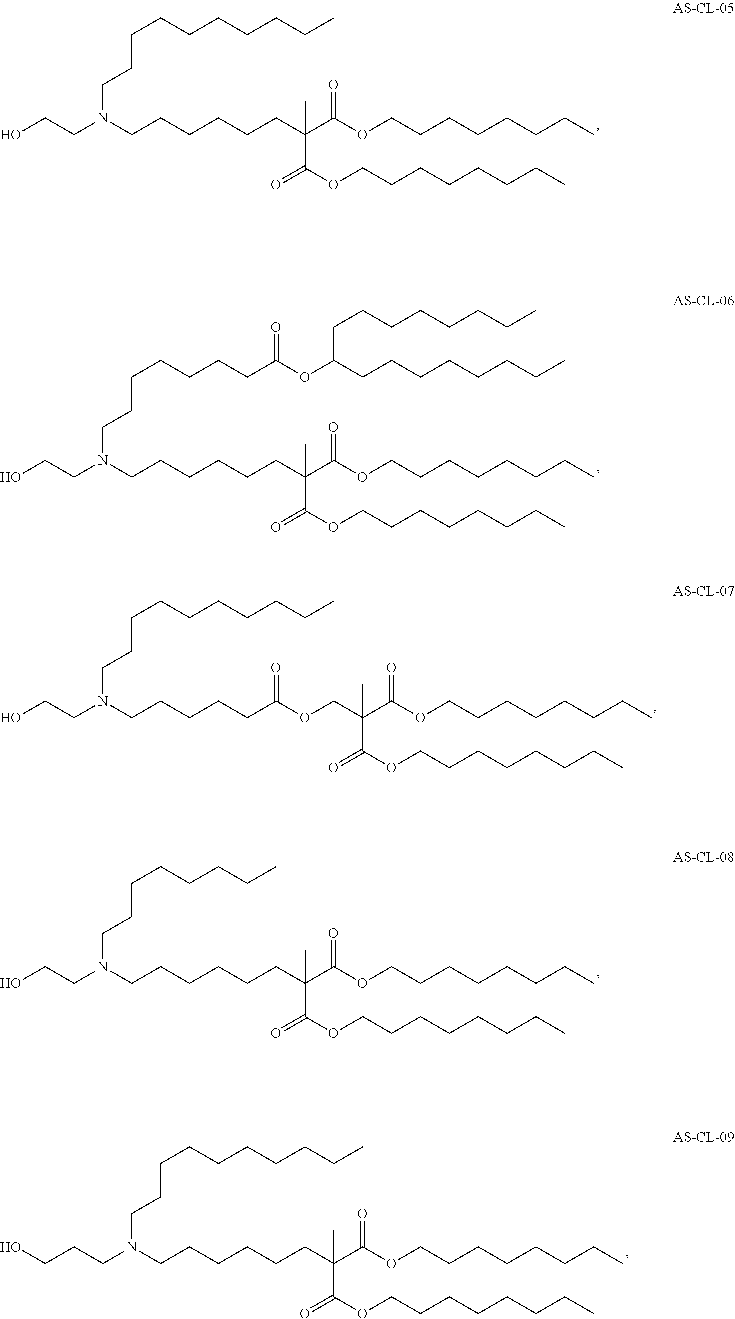 [SMILES: CCCCCCCCCCN(CCO)CCCCCCC(C)(C(=O)OCCCCCCCC)C(=O)OCCCCCCCC ; CCCCCCCCOC(=O)C(C)(CCCCCCN(CCO)CCCCCCCC(=O)OC(CCCCCCCC)CCCCCCCC)C(=O)OCCCCCCCC ; CCCCCCCCCCN(CCO)CCCCCC(=O)OCC(C)(C(=O)OCCCCCCCC)C(=O)OCCCCCCCC ; CCCCCCCCOC(=O)C(C)(CCCCCCN(CCO)CCCCCCCC)C(=O)OCCCCCCCC ; CCCCCCCCCCN(CCCO)CCCCCCC(C)(C(=O)OCCCCCCCC)C(=O)OCCCCCCCC] 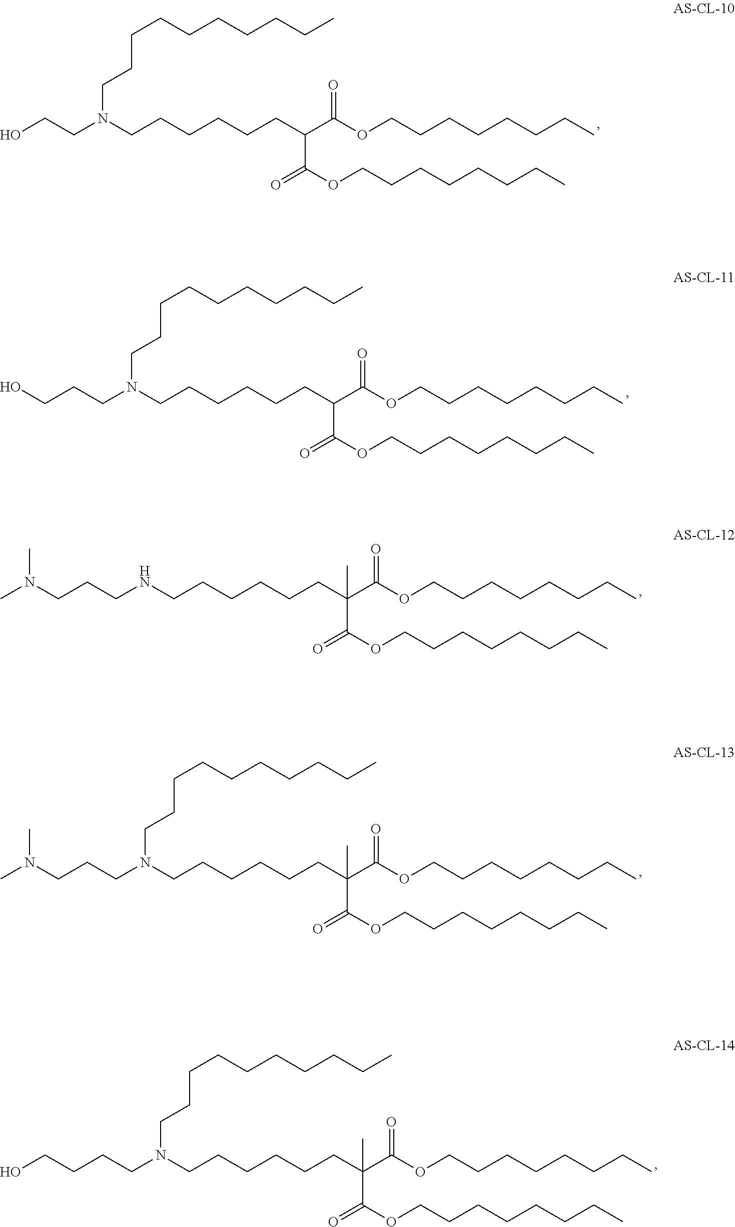 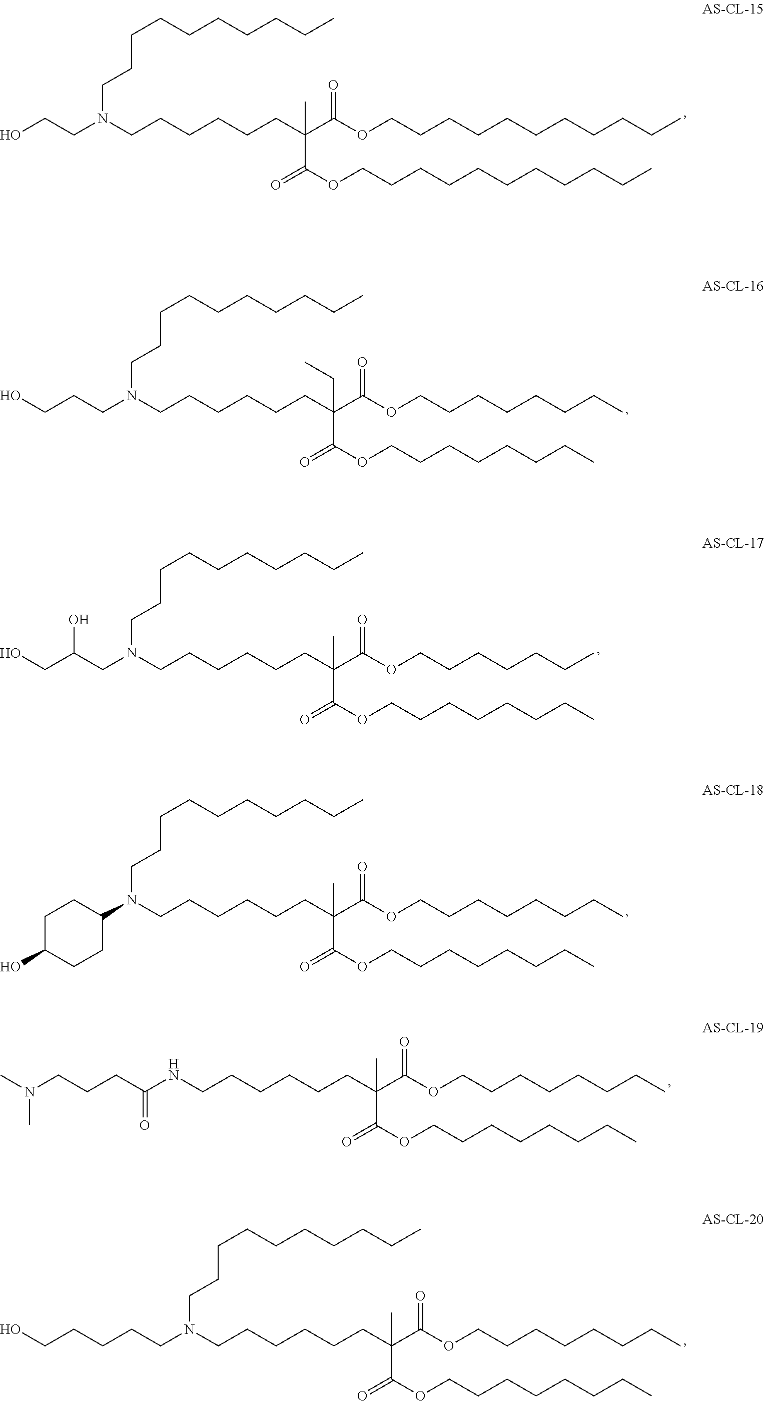 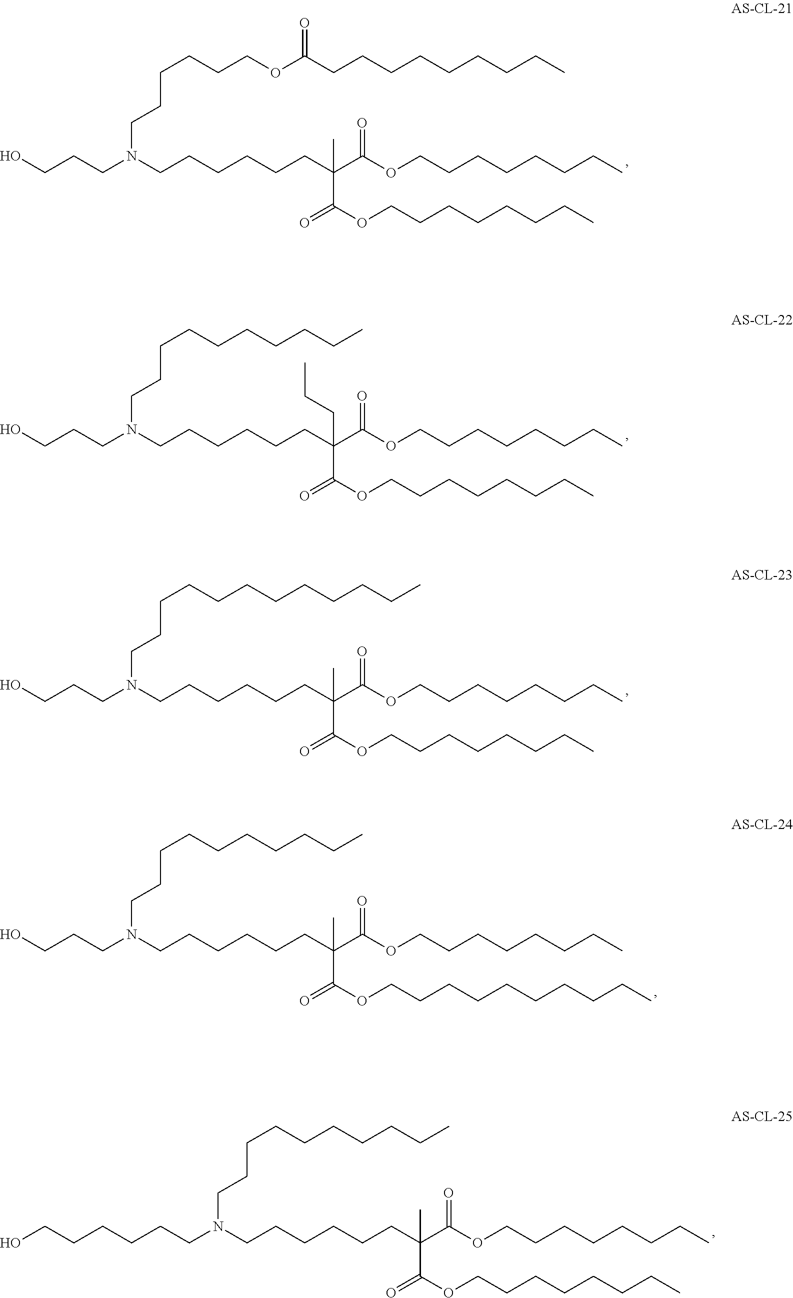 [SMILES: CCCCCCCCCC(=O)OCCCCCCN(CCCO)CCCCCCC(C)(C(=O)OCCCCCCCC)C(=O)OCCCCCCCC ; CCCCCCCCCCN(CCCO)CCCCCCC(CCC)(C(=O)OCCCCCCCC)C(=O)OCCCCCCCC ; CCCCCCCCCCCCN(CCCO)CCCCCCC(C)(C(=O)OCCCCCCCC)C(=O)OCCCCCCCC ; CCCCCCCCCCOC(=O)C(C)(CCCCCCN(CCCO)CCCCCCCCCC)C(=O)OCCCCCCCC ; CCCCCCCCCCN(CCCCCCO)CCCCCCC(C)(C(=O)OCCCCCCCC)C(=O)OCCCCCCCC] 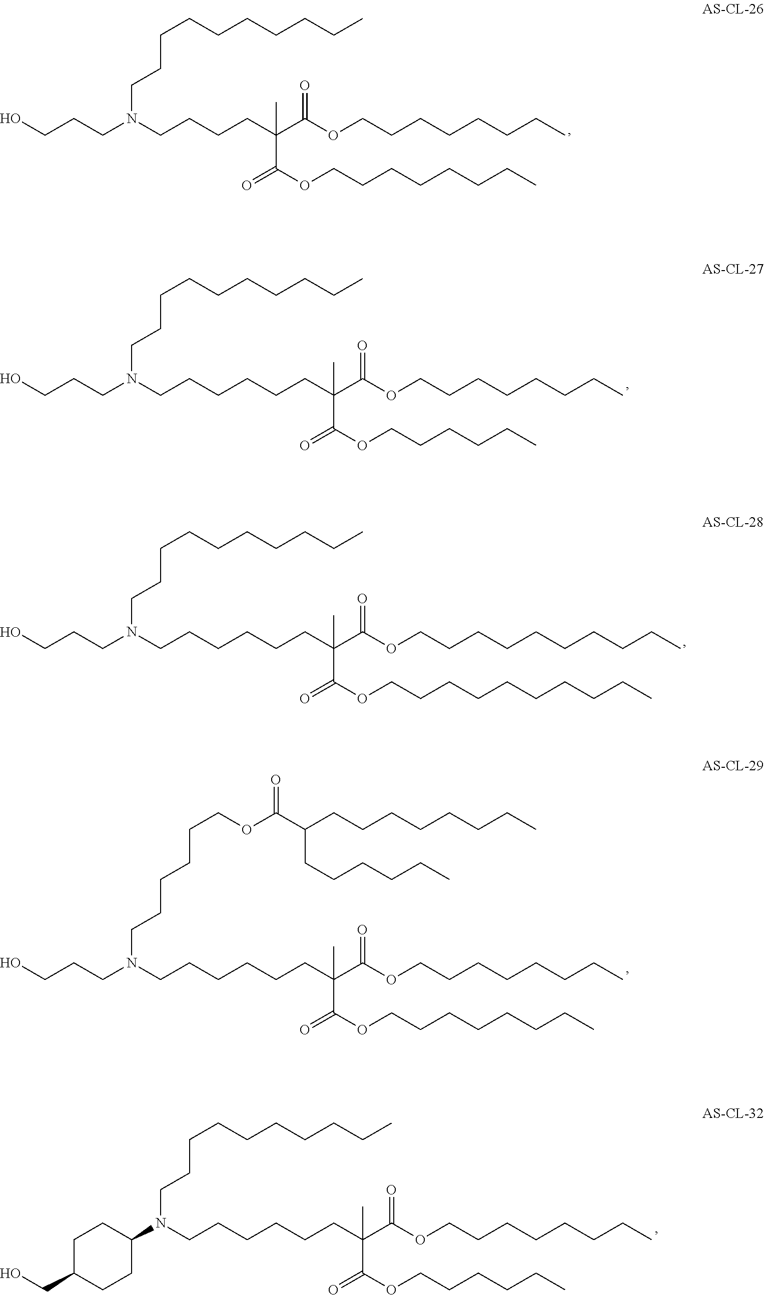 [SMILES: CCCCCCCCCCN(CCCO)CCCCC(C)(C(=O)OCCCCCCCC)C(=O)OCCCCCCCC ; CCCCCCCCCCN(CCCO)CCCCCCC(C)(C(=O)OCCCCCC)C(=O)OCCCCCCCC ; CCCCCCCCCCOC(=O)C(C)(CCCCCCN(CCCO)CCCCCCCCCC)C(=O)OCCCCCCCCCC ; CCCCCCCCOC(=O)C(C)(CCCCCCN(CCCO)CCCCCCOC(=O)C(CCCCCC)CCCCCCCC)C(=O)OCCCCCCCC ; CCCCCCCCCCN(CCCCCCC(C)(C(=O)OCCCCCC)C(=O)OCCCCCCCC)[C@H]1CC[C@@H](CO)CC1] 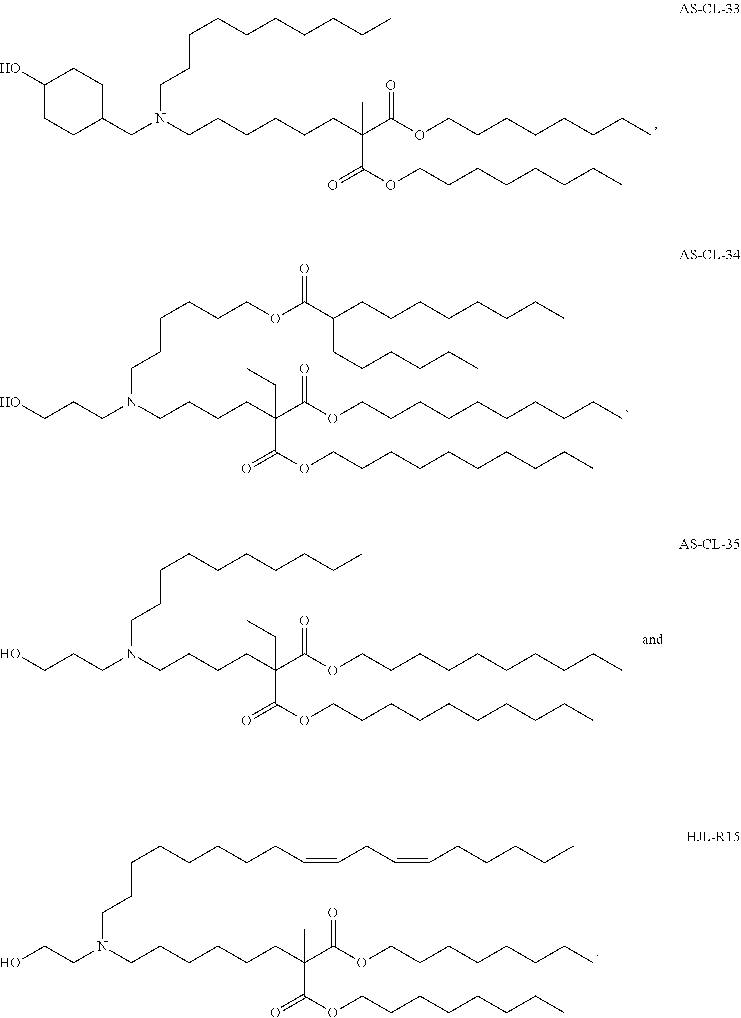 [SMILES: CCCCCCCCCCN(CCCCCCC(C)(C(=O)OCCCCCCCC)C(=O)OCCCCCCCC)CC1CCC(O)CC1 ; CCCCCCCCCCOC(=O)C(CC)(CCCCN(CCCO)CCCCCCOC(=O)C(CCCCCC)CCCCCCCC)C(=O)OCCCCCCCCCC ; CCCCCCCCCCOC(=O)C(CC)(CCCCN(CCCO)CCCCCCCCCC)C(=O)OCCCCCCCCCC ; CCCCC/C=C\C/C=C\CCCCCCCCN(CCO)CCCCCCC(C)(C(=O)OCCCCCCCC)C(=O)OCCCCCCCC]
